(19) United States
(12) Patent Application Publication
(10) Pub. No.: US 2024/0301093 A1
(43) Pub. Date: Sep. 12, 2024

(54) TRIAZOLE CONTAINING POLYMERS AND METHODS OF USE THEREOF

(71) Applicant: William Marsh Rice University, Houston, TX (US)

(72) Inventors: David ZHANG, Houston, TX (US); Ping SONG, Houston, TX (US); Omid VEISEH, Houston, TX (US); Siavash PARKHIDEH, Houston, TX (US); Sudip MUKHERJEE, Houston, TX (US); Maria Isabel RUOCCO, Houston, TX (US); Boram KIM, Houston, TX (US); Michael David DOERFERT, Houston, TX (US); Yuxuan CHENG, Houston, TX (US)

(73) Assignee: William Marsh Rice University, Houston, TX (US)

(21) Appl. No.: 18/569,673

(22) PCT Filed: Jun. 14, 2022
(86) PCT No.: PCT/US2022/033416

§ 371 Date: Dec. 13, 2023

Related U.S. Application Data
(60) Provisional application No. 63/210,377, filed on Jun. 14, 2021.

(51) Int. Cl. C08B 37/00; A61K 9/48; A61K 35/39; A61K 35/51; A61L 27/34; A61L 27/38; A61L 29/08; C12Q 1/6881

(52) U.S. Cl. CPC C08B 37/0084; A61K 9/4816; A61K 35/39; A61K 35/51; A61L 27/34; A61L 27/3804; A61L 29/085; C12Q 1/6881; A61L 2400/02

(57) ABSTRACT

Disclosed herein are compounds of the formula: A-L-R₁(I), wherein the these variables are defined herein, as well as medical devices comprising said compounds. The present disclosure also provides pharmaceutical compositions comprising the compounds or medical devices disclosed herein. Further, the present disclosure provides methods of treatment using the compounds, medical devices, or pharmaceutical compositions disclosed herein.

### CROSS REFERENCE TO RELATED APPLICATIONS

This application claims the benefit of priority to U.S. Provisional Application No. 63/210,377, filed Jun. 14, 2021, which is hereby incorporated by reference in its entirety.

### STATEMENT REGARDING FEDERALLY FUNDED RESEARCH

This invention was made with government support under Grant No. R01DK120459, awarded by the National Institutes of Health. The government has certain rights in the invention.

### BACKGROUND OF THE INVENTION

### I. Field of the Invention

The present invention relates generally to the fields of biology, chemistry, and medicine. More particularly, it concerns compounds, compositions and methods for the treatment and prevention of diseases and disorders such as those associated with fibrosis.

### II. Description of Related Art

Diabetes, hemophilia, mucopolysaccharidosis and many other diseases can be managed or suppressed through protein drugs, such as insulin or monoclonal antibodies or the recent emerging cell-based therapies. However, protein drugs cannot be administered orally, and must be injected intravenously (IV). Protein drugs are quickly degraded, limiting therapeutic effect and requiring regular IV injections to maintain therapeutic levels in the body. The burden of regular IV injections is a major limitation to patient quality of life and results in high healthcare costs.

Cells implanted into the human body can in principle act as a “living factory” and constantly produce the protein drug. However, the patient's immune system will destroy these foreign implanted cells, so some mechanism for encapsulating the cells within non-immunogenic materials is needed to allow long-term cell therapy. The field of cell therapy has been limited for a long time because biomaterials for encapsulating cells induce a foreign body response, i.e. fibrosis. This fibrosis encases the implant, limiting transfer of oxygen and nutrients into the encapsulated cells, and leads to cell death. Hence it is of an urgent medical need to develop an adequately biocompatible transplantation device or an immunoprotective coating that can prevent host immune recognition and delay the fibrosis

Previous studies demonstrated the use of a high-throughput hydrogel library to screen a library of covalently modified triazole containing alginate analogues to identify lead biomaterials effective to prevent against fibrosis in rodent as well as non-human primate model (Vegas et al., 2016). Screening a total of 774 combinatorially synthesized chemicals led to identifying three lead triazole containing anti-fibrotic small molecule compounds with similar molecular structure (Vegas et al., 2016). For the in vivo evaluation of such alginate encapsulation materials, eight different subcutaneous implantation sites were used in each mouse. However, it is important to highlight that the high-throughput screening of novel biomaterials involves a rigorous use of living object (mice/non-human primates: NHP) that significantly increase the time, cost and compromises a high number of rodents/non-human primates. However, the number of samples tested is very small and no consistent high throughput screening methods were developed to screen large numbers of immunoprotective biomaterials in a single rodent/NHP which will help to reduce the number of the experimental living objects. As such, there exists a need for additional triazole compounds as well as high throughput screening methods to identify immunoprotective biomaterials.

The development of this invention was funded in part by the Cancer Prevention and Research Institute of Texas Grant No. RR160047.

### SUMMARY OF THE INVENTION

The present disclosure provides triazole containing compounds with anti-fibrotic properties, pharmaceutical compositions, methods for their manufacture, and methods for their use.

In one aspect, there are provided compounds of the formula:

A-L-R₁ (I),

wherein:
A is a polymer;
L is a linker of the formula:

NRₐX₁(CH₂CH₂O)ₒ
wherein:
   Rₐ is hydrogen, alkyl_{(C≤6)}, or substituted alkyl_{(C≤6)};
   is 2, 3, 4, or 5; and
   X₁ is alkanediyl_{(C≤8)} or substituted alkanediyl_{(C≤8)};
or a linker of the formula:

NR_{b}(CH₂)ₚX₂
wherein:
   R_{b} is hydrogen, alkyl_{(C≤6)}, or substituted alkyl_{(C≤6)};
   p is 1, 2, or 3; and
   X₂ is arenediyl_{(C≤12)} or substituted arenediyl_{(C≤12)};
R₁ is a cycloalkyl_{(C≤12)}; haloaryl_{(C≤12)}; S containing heteroaryl_{(C≤12)}; substituted S-containing heteroaryl_{(C≤12)}; alkyl_{(C≤6)}, haloalkyl_{(C≤6)}, alkenyl_{(C≤6)}, or alkynyl_{(C≤6)} substituted aryl_{(C≤12)}; aralkyl_{(C≤12)}; substituted aralkyl_{(C≤12)}; heterocycloalkyl_{(C≤12)}; substituted heterocycloalkyl_{(C≤12)}; 2-pyridinyl; 3-aminophenyl; 4-alkoxy_{(C≤6)} substituted aryl_{(C≤12)}; or a group of the formula:

X₃OR₂
wherein:
   X₃ is alkanediyl_{(C≤8)} or substituted alkanediyl_{(C≤8)};
   R₂ is aryl_{(C≤12)} or substituted aryl_{(C≤12)};
      or a pharmaceutically acceptable salt thereof.

In some embodiments, the compounds are further defined as:

A-L-R₁ (I)

wherein:
A is a polymer
L is a linker of the formula:

NRₐX₁(CH₂CH₂O)ₘ
wherein:
   Rₐ is hydrogen, alkyl_{(C≤6)}, or substituted alkyl_{(C≤6)};
   m is 2, 3, 4, or 5; and
   X₁ is alkanediyl_{(C≤8)} or substituted alkanediyl_{(C≤8)};
R₁ is a cycloalkyl_{(C≤12)}; haloaryl_{(C≤12)}; S containing heteroaryl_{(C≤12)}; substituted S-containing heteroaryl_{(C≤12)}; alkyl_{(C≤6)}, haloalkyl_{(C≤6)}, alkenyl_{(C≤6)}, or alkynye_{(C≤6)} substituted aryl_{(C≤12)}; 3-aminophenyl; 4-alkoxy_{(C≤6)} substituted aryl_{(C≤12)}; or a group of the formula:

X₃OR₂
wherein:
   X₃ is alkanediyl_{(C≤8)} or substituted alkanediyl_{(C≤8)};
   R₂ is aryl_{(C≤12)} or substituted aryl_{(C≤12)};
      or a pharmaceutically acceptable salt thereof.
      In some embodiments, the compounds are further defined as:

A-L-R₁ (I)

wherein:
A is a polymer
L is a linker of the formula:

NRₐX₁(CH₂CH₂O)ₘ
wherein:
   Rₐ is hydrogen, alkyl_{(C≤6)}, or substituted alkyl_{(C≤6)};
   m is 2, 3, 4, or 5; and
   X₁ is alkanediyl_{(C≤8)} or substituted alkanediyl_{(C≤8)};
or a linker of the formula:

NR_{b}(CH₂)ₙX₂
wherein:
   R_{b} is hydrogen, alkyl_{(C≤6)}, or substituted alkyl_{(C≤6)};
   n is 1, 2, or 3; and
   X₂ is arenediyl_{(C≤12)} or substituted arenediyl_{(C≤12)};
R₁ is a haloaryl_{(C≤12)}; aralkyl_{(C≤12)}; substituted aralkyl_{(C≤12)}; heterocycloalkyl_{(C≤12)}; substituted heterocycloalkyl_{(C≤12)}; 2-pyridinyl; 3-aminophenyl;
   or a pharmaceutically acceptable salt thereof.

In some embodiments, the polymer comprises one or more sugar repeating units such as the repeating unit has a formula:

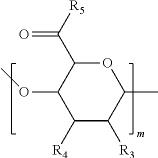

wherein:
R₃ or R₄ are each independently hydrogen or hydroxy;
R₅ is a hydroxy, alkoxy_{(C≤8)}, substituted alkoxy_{(C≤8)}, or a covalent bond to the linker; and
m is a number of repeating units with a molecular weight from about 50,000 Daltons to about 500,000 Daltons.

In some embodiments the polymer comprises repeating units of the formula:

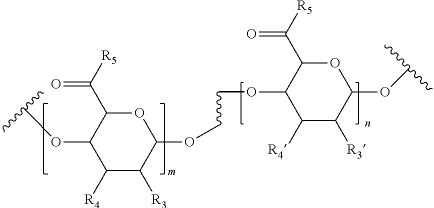

wherein:
R₃, R₃′, R₄, or R_{4′} are each independently hydrogen or hydroxy;
R₅ is a hydroxy, alkoxy_{(C≤8)}, substituted alkoxy_{(C≤8)}, or a covalent bond to the linker;
R_{5′} is a covalent bond to the linker; and
m and n result in a number of repeating units with a molecular weight from about 50,000 Daltons to about 500,000 Daltons.

In some embodiments, the polymer is an acrylate polymer such as a methacrylate polymer. In some embodiments, o is 2 o 3. In some embodiments, o is 2. In other embodiments, o is 3. In some embodiments, Rₐ is hydrogen. In some embodiments, X₁ is alkanediyl_{(C≤6)} such as —CH₂CH₂—. In some embodiments, R_{b} is hydrogen. In some embodiments, p is 1 or 2. In some embodiments, p is 1. In some embodiments, X₂ is arenediyl_{(C≤12)} such as benzenediyl.

In some embodiments, R₁ is haloaryl_{(C≤12)} such as chlorophenyl, bromophenyl, or fluorophenyl. In some embodiments, R₁ is 2-bromophenyl, 4-chlorophenyl, 2-fluorophenyl, or 4-fluorophenyl. In other embodiments, R₁ is 3-aminophenyl. In other embodiments, R₁ is an S containing heteroaryl_{(C≤12)} or substituted S containing heteroaryl_{(C≤12)}. In some embodiments, R₁ is S containing heteroaryl_{(C≤12)} such as 2-thiophenyl or 3-thiophenyl. In other embodiments, R₁ is cycloalkyl_{(C≤12)} such as cyclopropyl. In other embodiments, R₁ is alkyl_{(C≤6)}, haloalkyl_{(C≤6)}, alkenyl_{(C≤6)}, or alkynye_{(C≤6)} substituted aryl_{(C≤12)}. In some embodiments, R₁ is alkyl_{(C≤6)} substituted aryl_{(C≤12)} such as 3-methylphenyl or 4-methylphenyl. In other embodiments, R₁ is a haloalkyl_{(C≤6)} substituted aryl_{(C≤12)} such as 4-trifluoromethylphenyl. In other embodiments, R₁ is an alkynye_{(C≤6)} substituted aryl_{(C≤12)} such as 3-ethyne-phenyl. In some embodiments, R₁ is a group of the formula:

X₃OR₂

wherein:
X₃ is alkanediyl_{(C≤8)} or substituted alkanediyl_{(C≤8)};
R₂ is aryl_{(C≤12)} or substituted aryl_{(C≤12)}.
   In some embodiments, X₃ is alkanediyl_{(C≤8)} such as —CH₂—. In some embodiments, R₂ is substituted aryl_{(C≤12)} such as 4-aminophenyl. In other embodiments, R₁ is 4-alkoxy_{(C≤6)} substituted aryl_{(C≤12)} such as 4-ethoxyphenyl. In other embodiments, R₁ is heterocycloalkyl_{(C≤12)} or substituted heterocycloalkyl_{(C≤12)}. In some embodiments, R₁ is heterocycloalkyl_{(C≤12)} such as thiomorpholine-dioxide. In other embodiments, R₁ is aralkyl_{(C≤12)} or substituted aralkyl_{(C≤12)}. In some embodiments, R₁ is aralkyl_{(C≤12)} such as 2-phenylethyl. In some embodiments, the compounds are further defined as:

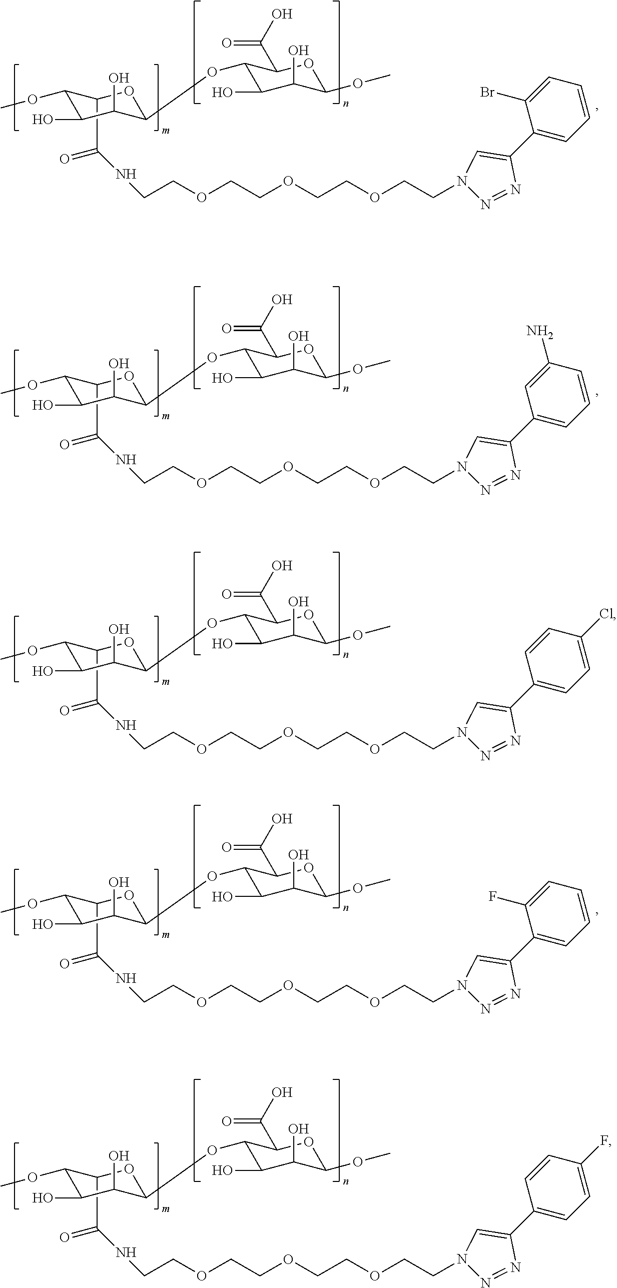

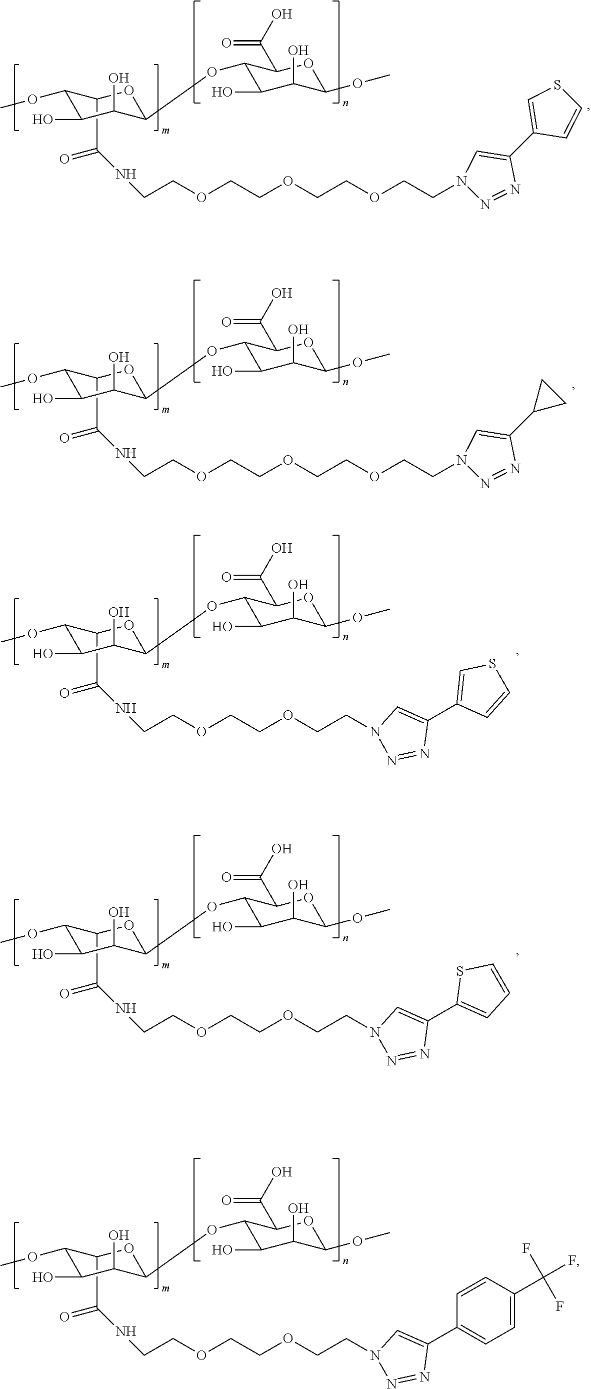

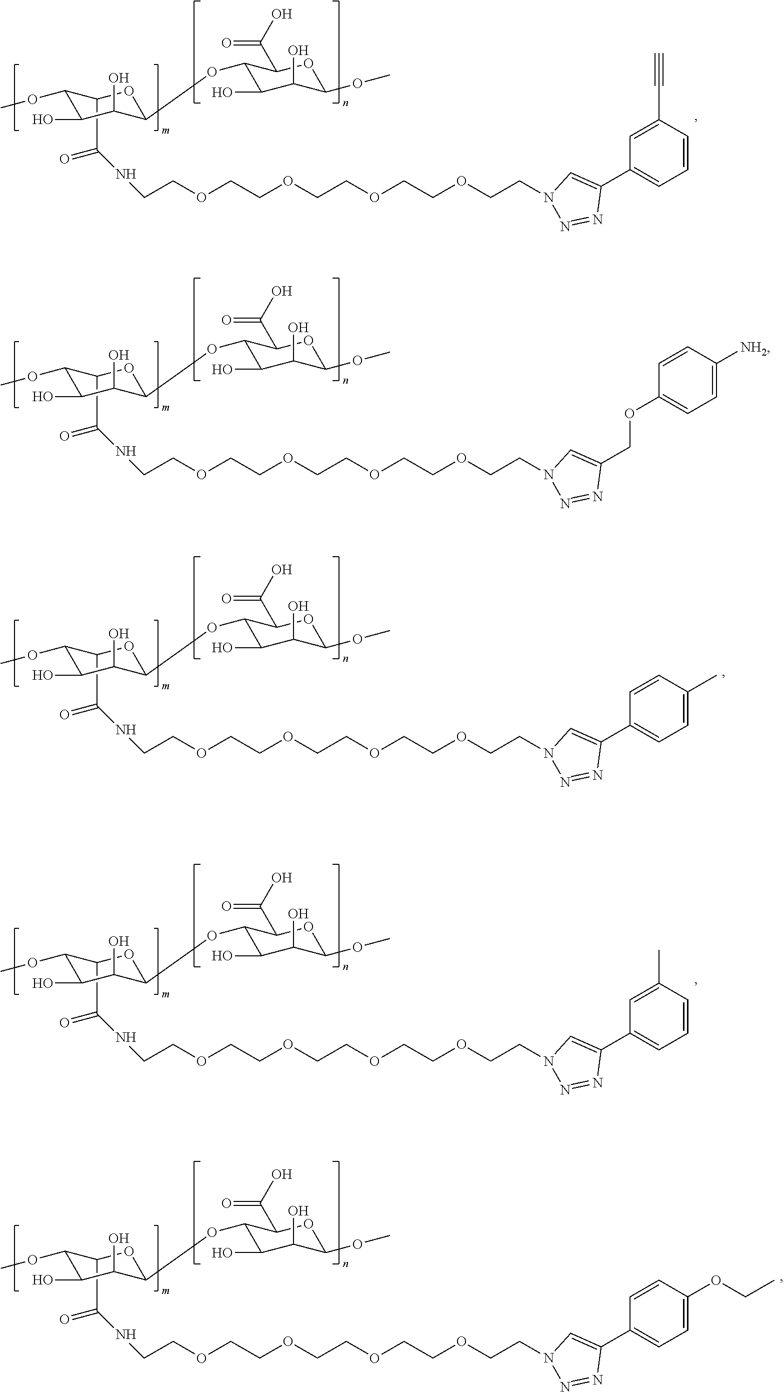

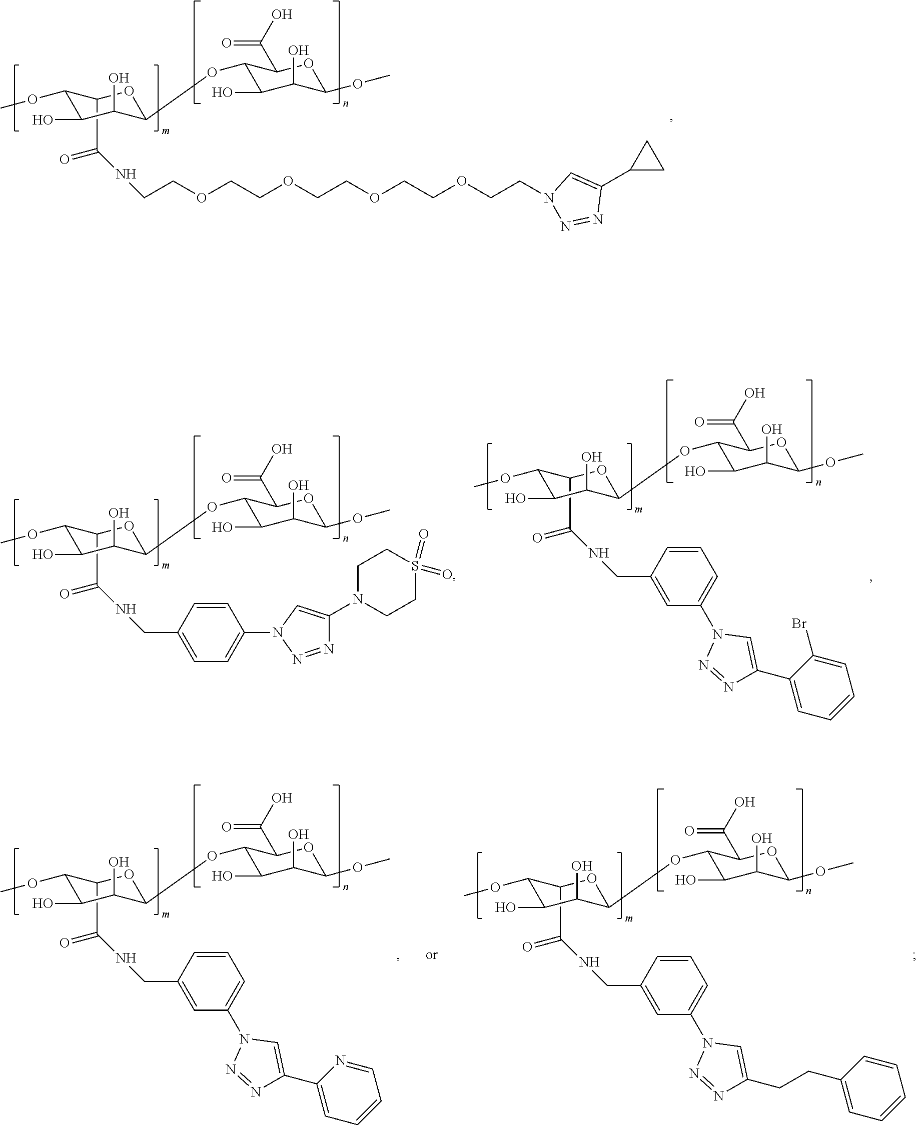

m and n result in a number of repeating units with a molecular weight from about 50,000 Daltons to about 500,000 Daltons.

In still another aspect, the present disclosure provides methods of detecting fibrosis in a sample comprising exposing the sample to one or more polymers described herein and measuring reactivity.

In another aspect, the present disclosure provides medical devices, wherein the medical device is coated with a compound described herein. In some embodiments, the medical devices are an implantable device, a cardiac pacemaker, a catheter, a needle injection catheter, a blood clot filter, a vascular transplant, a balloon, a stent transplant, a biliary stent, an intestinal stent, a bronchial stent, an esophageal stent, a ureteral stent, an aneurysm-filling coil or other coil device, a surgical repair mesh, a breast implant, a silicone implant, PDMS, a transmyocardial revascu-larization device, a percutaneous myocardial revasculariza-tion device, a prosthesis, an organ, a vessel, an aorta, a heart valve, a tube, an organ replacement part, an implant, a fiber, a hollow fiber, a membrane, a textile, banked blood, a blood container, a titer plate, an adsorber media, a dialyzer, a connecting piece, a sensor, a valve, an endoscope, a filter, a pump chamber, or another medical device intended to have hemocompatible properties or used in cancer, diabetes, ischemia, anti-bacterial, hemophilia, stroke, blood disorder, or a cytokine therapy involving human engineered cells.

In some embodiments, the medical devices are a capsule, an implantable polymer block, 3D printed block, 3D printed gel, or a polymer encapsulating device. In some embodiments, the polymer encapsulating device further comprises a shape selected from spheres, squares, noodles, needles, rectangles, and cylindrical. In some embodiments, the implantable capsule is a microcapsule. In some embodiments, the medical devices are a catheter. In some embodiments, the medical devices result in less fibrosis than a medical device without the coating. In some embodiments, the medical devices are immunoprotective compared to a medical device without the coating. In some embodiments, the immunoprotective results in a lower foreign body response.

In still another aspect, the present disclosure provides pharmaceutical compositions comprising:
(A) a compound or medical device described herein; and
(B) an excipient.

In some embodiments, the pharmaceutical compositions further comprise biological material. In some embodiments, the biological material is encapsulated in the compound or medical device. In some embodiments, the biological material is cells. In some embodiments, the cells are cells from xenotissue, cells from a cadaver, stem cells, cells derived from stem cells, cells from a cell line, primary cells, reprogrammed cells, reprogrammed stem cells, cells derived from reprogrammed stem cells, genetically engineered cells, or a combination thereof. In some embodiments, the cells are human cells. In some embodiments, the cells are insulin-producing cells. In some embodiments, the cells are pancreatic islet cells. In some embodiments, the compound is cross-linked. In some embodiments, the cross-linked compound is covalently cross-linked.

In still another aspect, the present disclosure provides methods of treating or preventing a disease or disorder comprising administering to a patient in need thereof a compound, a medical device, or a pharmaceutical composition described herein. In some embodiments, the methods result in a lower foreign body response. In some embodiments, the methods result in less fibrosis.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description. Note that simply because a particular compound is ascribed to one particular generic formula doesn't mean that it cannot also belong to another generic formula.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a general schematic outlining the synthesis of new alginate analogues. The specific linkers and alkynes used are shown in Table 2. Left of the scheme are two of the previous triazole-containing lead alginates (B1-A21 and Z1-A34) that prevent fibrosis in mouse and NHP models. Total 211 new alginate analogs were synthesized by varying the lead hydrophilic linker (Azido PEG-amine) and hydrophobic linker (iodo benzyl amine) combined with a set of alkyne classes shown at center.

FIGS. 2A-2D show cell barcoding strategy for materials screening. (FIG. 2A) Overall schematic diagram: 20 different HUVEC donors were encapsulated with corresponding materials and implanted into mice for evaluating anti-fibrotic property and biocompatibility for 4 weeks. (FIG. 2B) Twenty unique HUVECs have been sequenced via NGS to identify their specific SNPs, which can be used as a barcode to tag and identify different encapsulation materials in vivo. (20) unique HUVECs have been sequenced via next-generation sequencing (NGS) to identify their individual single nucleotide polymorphisms (SNPs) which can be used as a barcode to tag and identify different encapsulation materials in vivo. (FIGS. 2C, 2D) To prove all donors (H1-H20) can be deconvoluted by NGS, a mixture of 20 different capsules encapsulating 20 different HUVECs donors were implanted in NSG mice for four weeks. (FIG. 2C) Bright-field and dark-field images of the pre-implant and post-implant; capsules were retrieved with minimal cell deposition, indicating no fibrosis, similar to pre-implant capsules. Encapsulated cells are still alive (live cells: green, dead cells: red) after 4 weeks of implantation. Scale bar, 2 mm. (FIG. 2D) The capsules from one mouse were analyzed with NGS, and 195 out of 200 capsules were successfully identified. Across the donors, the identified capsules percent are evenly distributed from donor 1 to donor 20.

FIGS. 3A-3E show gelation assay results and material characterization. (FIG. 3A) Gelation assay of alginate analogues using Rhodamine B entrapment. (FIG. 3B) Representative images of capsule formation using the alginate analogs. Scale bar, 2 mm. (FIG. 3C) After initial characterization studies, including purity, solubility, and gel-forming ability, 149 alginate polymers were used for the screening test. (FIG. 3D) Table for elemental analysis of alginate linkers confirming the linker conjugation to the alginate backbone. (FIG. 3E) Representative NMR image to show the triazole peak in alginate analogues confirming the modifications.

FIGS. 4A-4C show optimization of DNA extraction method with pre-implant capsules. An optimized method was used for further analysis, such as qPCR and NGS identification with post-implant capsules. (FIG. 4A) Comparison of DNA content depending on capsule lysis conditions; with vs. without EDTA lysis steps and fresh vs. frozen conditions. (FIG. 4B) Comparison of DNA content depending on DNA elution conditions; elution volume and elution temperature. (FIG. 4C) Comparison of DNA content/yield depending on cell numbers per capsule.

FIGS. 5A & 5B show optimization of NGS library preparation workflow. The library preparation involved a multiplex PCR step in amplifying the SNP loci, a barcoding PCR step to add position barcode to each sample, and a ligation-based sequencing adapter amendment procedure. (FIG. 5A) Before optimization, the library on-target rate was <10%, with primer-dimers and non-specific PCR products contributing to the majority of reads. (FIG. 5B) After optimization, the on-target rate was increased to >80% regardless of low DNA input (<1 ng) in the starting material.

FIG. 6 shows the bioinformatic pipeline for determining material identity/composition from NGS sequencing data. Fastq NGS data was demultiplexed by row and column barcodes to re-group sequences amplified from the same DNA input. Then for each amplicon sequence, the grep function was applied to search the dominant and variant alleles to calculate variant allele frequency (VAF) for ach SNP locus. If the encapsulated cells comprised only one donor, the VAF profile was compared against profiles of the 20 pre-screened HUVEC donors. The donor with the highest match rate was identified as the encapsulated donor cell. When one or two donors were used as encapsulated cells, the log-likelihood of all possible donor compositions was calculated. The composition with the highest overall log-likelihood was determined as the cell composition (Quality control for log-likelihood analysis: 1) at least 25/30 SNP loci had sequencing coverage >50; and 2) overall log-likelihood higher than −200, and 3) goodness measurement higher than 10 where goodness is defined as the difference of log-likelihood between the most likely and the second most likely donor pairs). The material corresponding to the identified donor cell or cell composition would be the material encapsulating cells.

FIGS. 7A-7F show high-throughput screening of combinatorially synthesized chemically modified alginates using unique cellular barcoding facilitates identifying new hydrogels with reduced fibrosis in immune-competent mice. (FIG. 7A) A library of immunomodulatory biomaterials; total 211 novel alginate analogs were synthesized. (FIG. 7B) The mixtures of different materials were implanted in the same implantation site to increase screening throughput. NGS assay was used to determine material identity by demultiplexing the SNP genotype of encapsulated HUVECs. Typical implantation of 200 alginate capsules (˜1.5 mm diameter, 10 capsules/material) allows the simultaneous evaluation of 20 different implanted materials, with sufficient independent capsules per material to enable statistical analysis (FIG. 7C) Representative results from one of the rounds. After four weeks of implantation, capsules were explanted. Clear capsules with low fibrosis (bottom row), similar to pre-implant capsules, were separated for further analysis. Scale bar, 10 mm. (FIG. 7D) Heat map summarizing material screening for the entire alginate analogs. (FIG. 7E) 149 new materials were screened, and corresponding lead materials were identified via NGS assay. Error bars represent 95% confidence intervals from a binomial distribution. The average level of Z1-A34 (previous positive material, labeled as a bar) was marked as a dotted line. Top materials over Z1-A34 were labeled with a dark bar. (FIG. 7F) Representative structures of top three lead alginate analogs (orange bars in FIG. 7E).

FIGS. 8A-811 show the scale up of materials screening in the NHP model using dual-donors barcoding. (FIG. 8A) Two HUVEC donors were mixed at a 1:2 ratio and encapsulated in various materials. (FIG. 8B) With 20 HUVEC donors mixed at the ratio of 1:2, 20×20=400 distinct SNP profiles were created. (FIG. 8C) 100 donor pairs were encapsulated with corresponding materials and implanted into IP space in an NHP for four weeks. Thirty capsules per material were used, and a total of 3000 capsules were implanted. (FIG. 8D) The representative images of preimplant capsules. (FIG. 8E) After four weeks, all the free-floating capsules in IP space were collected and used for material identification (light arrow: capsules with fibrotic tissue aggregation, gray arrow: free-floating capsules). (FIG. 8F) Summary of donor pair identification. Among a total of 503 selected capsules, 466 (92.6%) were identified with high confidence, 32 (6.36%) with lower confidence, and 5 (0.99%) capsules failed to be identified. (FIG. 8G) Distribution of confidence level of analyzed capsules. Goodness is the difference between the log-likelihood of most likely pair and second most likely donor pair, and thus high goodness indicates a low chance of misidentification. Capsules at the upper right corner have higher confidence, and those with log-likelihood below −200 or goodness less than 10 are considered “less confident”. (FIG. 8H) The chemical structure of top 4 identified leads.

FIGS. 9A-9D show optimization of two HUVEC donors barcoding for expanded barcoding capacity for larger library screening. (FIG. 9A) Mixture of three different materials containing corresponding donor pairs were tested in vitro. Goodness is the log-likelihood difference between the picked most likely pair and the second most likely pair, which is a measurement of how stand-out is the picked combination. (FIGS. 9B-9D) Representative heatmaps plot the log-likelihood of each 20×20 donor combination in different mixing ratios (b, 1:2, c, 1:3, d, 1:4). The darkness of each small square codes for the likelihood.

FIGS. 10A-10F show dual donor barcoding identification in C57BL/6J mice. (FIG. 10A) Three different materials were tested; UP-VLVG (control), B1-A51 (one of the negative materials), and Z1-A34 (one of the positive materials). (FIG. 10B) Schematic workflow of three materials screening containing mixed dual donors. (FIG. 10C and FIG. 10D) After two weeks of implant, capsules were retrieved from each mouse (M1-M3) and were separated into three groups depending on fibrosis levels. (FIG. 10E) Representative heatmap result of identified donor pair. (FIG. 10F) 39 mapped to Z1-A34 (positive control material), coded by H16:H14 at 1:2 ratio; 4 mapped to UP-VLVG coded by H6:H8 at 1:2 ratio; 0 sample mapped to B1-A51. Overalled 43/45 samples were mapped from the 400 donor SNP profile. Proportions of each material corresponded to donor pairs were plotted, and Z1-A34 showed the highest value indicating the best immune-protective properties.

FIGS. 11A-11C show that lead hydrogels show low fibrosis intraperitoneally in C57BL/6J mice. (FIG. 11A) Representative dark-field images of preimplant and explanted microcapsules (300˜400 μm size) retrieved from IP space after 2 weeks. Scale bar, 2 mm. (FIG. 11B) Representative confocal images of explanted microcapsules; Capsules were stained with CD68 (light), DAPI (gray), and α-SMA (dark) markers. (FIG. 11C) RT-qPCR analysis to compare RNA expression in different materials. Expression of fibrotic markers (α-SMA and Col1a1) were normalized to SLG20 (control). Two-way ANOVA with Bonferroni correction was used for statistical analysis (****P<0.0001, SLG20 control vs. others).

FIG. 12 shows the results of diabetic reversal study with lead material (Z4-A10). Capsules containing human islets were fabricated at final cell density with 4,000, 8,000, and 16,000 IEQ/alginate volume (mL). The final IEQ values in each capsule were 10, 20, and 40 IEQ per capsule, respectively. In each group, 500 μL, 250 μL, and 125 μL of capsules were implanted in IP space, containing total 2,000 IEQ per mouse.

FIGS. 13A-13H show that lead hydrogel encapsulating xenogeneic human islets demonstrates a diabetic reversal in immunocompetent C57BL/6J mice. (FIG. 13A) Representative images of pre-implant capsules. Z4-A10 capsules containing human islets at a density of 10 IEQ/capsule, 20 IEQ/capsule, and 40 IEQ/capsule, respectively. SLG20 capsule was used as a control material. Dithizone staining indicates viable islets within the capsule matrix. After encapsulation, islets show good viability (live: light, dead: dark). (FIG. 13B) Blood glucose levels for both Z4-A10 and SLG20 groups (4,000 IEQ/mL density) were monitored until mice were euthanized (****P<0.0001 (SLG20 vs. Z4-A10)). (FIG. 13C) IVGTT test with Z4-A10 capsule (4,000 IEQ/mL) implant group in diabetic mouse, and non-implant group in diabetic mice and non-diabetic mice (ns; not significant, ****P<0.0001 (all comparisons)). (FIG. 13D, FIG. 13E) Representative dark-field (FIG. 13D), and dithizone staining (red, FIG. 13E) images of explanted Z4-A10 and SLG20 capsules (4,000 IEQ/mL). (FIG. 13F) Human c-peptide measurements at 80 days post-transplantation (SLG20 vs. Z4-A10). (FIG. 13G, FIG. 13H) Blood glucose monitoring with high islets density groups: Z4-A10 capsules (FIG. 13G) and SLG20 capsules (FIG. 13H). Error bars denote mean±sem; Two-way ANOVA with Bonferroni multiple-comparison correction.

FIGS. 14A-14E shows lead immuno-protective small molecules were used to coat catheter tubing to provide immune protection in the subcutaneous space of C57BL/6 mice. (FIG. 14A) Chemical structures of the unmodified group or coated with either Met-Z1-A3 or Met-B2-A17. (FIG. 14B) XPS data for unmodified, Met-Z1A3, and Met-B2-A17 modified catheters showing wt % of small molecule-specific atoms, indicating successful coating. (FIG. 14C) ToF-SIMS data for unmodified, Met-Z1A3, and Met-B2-A17 modified catheters showing the area with normalized intensity (a.u.) by total ion intensity for the main peaks (CN—, Br—), indicating successful coating. (FIG. 14D) Representative histology images of the measured fibrotic capsule for unmodified and coated catheters. The thinner, less dense purple band of cells at the tissue-catheter interface of the coated catheters indicates a milder immune response. (FIG. 14E) Quantification of fibrotic capsule thickness for unmodified and coated catheters. One-way ANOVA with Bonferroni correction was used for statistical analysis (****P<0.0001, ***P<0.002).

FIGS. 15A-15E show material characterization and evaluation of catheters coated with lead molecules. (FIG. 15A, FIG. 15B) The total intensity of two main peaks analyzed by Tof-SIMS (FIG. 15A, CN— and FIG. 15B, Br—) were plotted to compare with the unmodified catheter. (FIG. 15C) Representative SEM images of unmodified and coated catheters. (FIG. 15D) Example of the measured deposited fibrotic capsule tissue. ImageJ was used to measure tissue deposition via the purple band of tissue adjacent to the catheter. (FIG. 15E) Representative H&E-stained sections for explanted catheters of each group. Scale bar, 2 mm.

### DESCRIPTION OF ILLUSTRATIVE EMBODIMENTS

Disclosed herein are new compounds and compositions with anti-fibrotic properties, methods for their manufacture, and methods for their use, including for the treatment and/or prevention of disease. These compounds are alginate derivatives (described as modified alginate compounds) which contain one or more triazole group that links a compound to the alginate backbone. These compounds possess one or more improved properties compared to other alginates known in the field such as improved compatibility or activity in an in vivo or in vitro assay.

The present invention describes small molecules and small molecule-polymer conjugates which (1) have an anti-fibrotic property and (2) maintain encapsulated cell viability. The chemical structures of these materials are based on anti-fibrotic small molecules identified by an initial screen of roughly 700 materials (Vegas et al., 2016). Studies to date suggest that identified triazole compounds with immunomodulatory properties appear to occupy a privileged structure space whose immunomodulatory performance could not have been anticipated without performing the screen described herein. The triazole-containing modifications associated with improved in vivo performance overall suggest the structural analogs around these triazole modifications may be a versatile chemical space for designing biomaterials that can mitigate foreign body responses and modulate immune responses.

### I. Compounds of the Present Invention

The compounds of the present invention (also referred to as “modified alginate compounds” “compounds of the present disclosure” or “compounds disclosed herein”) are shown, for example, above, in the summary of the invention section, and in the claims below. They may be made using the synthetic methods outlined in the Examples section. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

TABLE 1
Examples of the Modified Alginate Compounds Described Herein

| Compound ID | Structural Formula |
|---|---|
| Z1A3 | 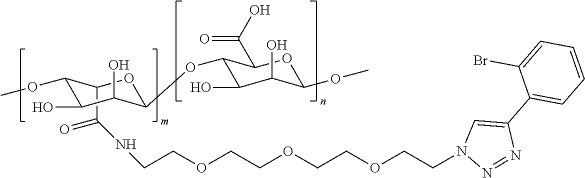 |
| Z1A5 | 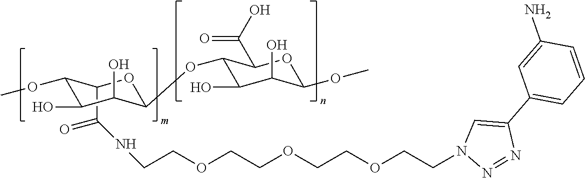 |
| Z1A7 | 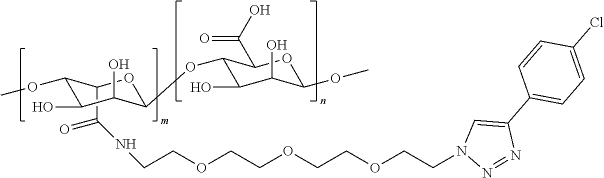 |
| Z1A14 | 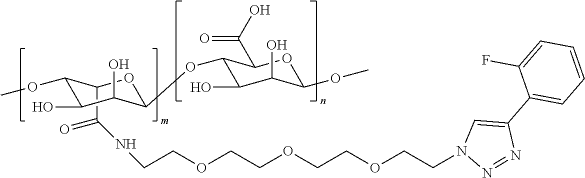 |
| Z1A16 | 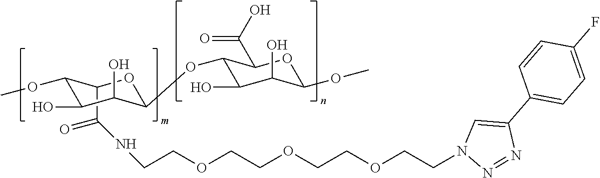 |
| Z1A19 | 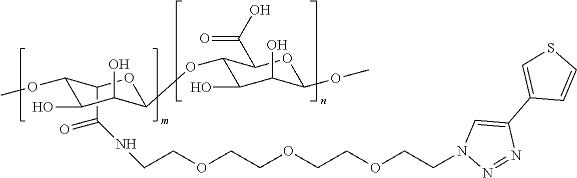 |
| Z1A43 | 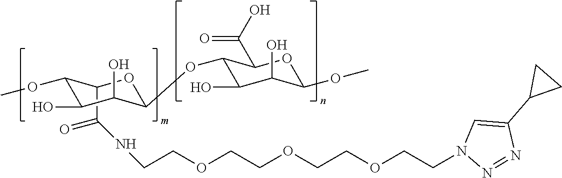 |
| Z2A19 | 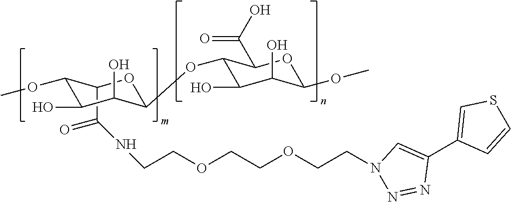 |
| Z2A20 | 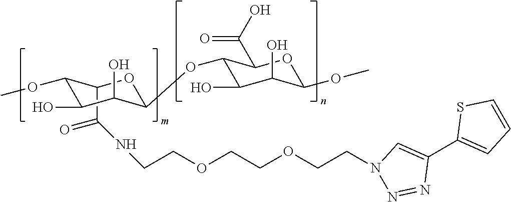 |
| Z2A28 | 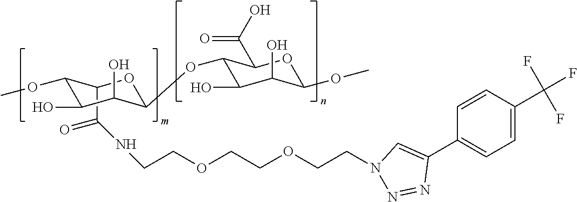 |
| Z3A10 | 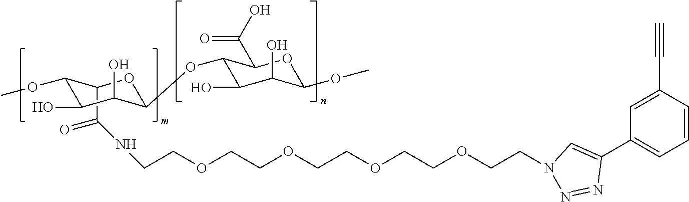 |
| Z3A22 | 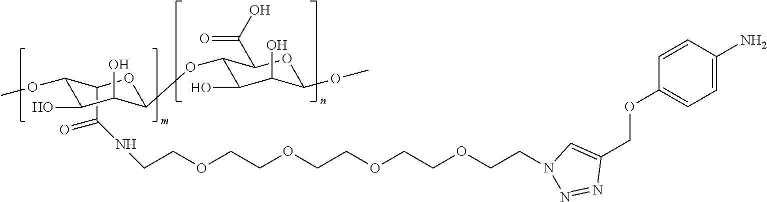 |
| Z3A26 | 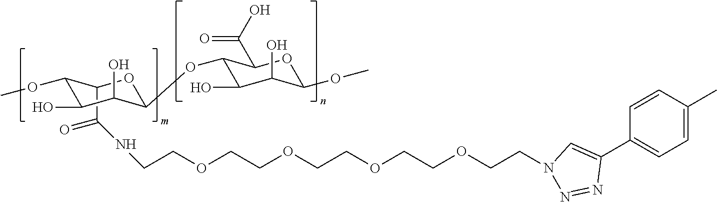 |
| Z3A27 | 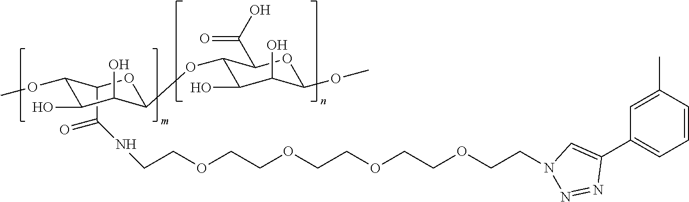 |
| Z3A30 | 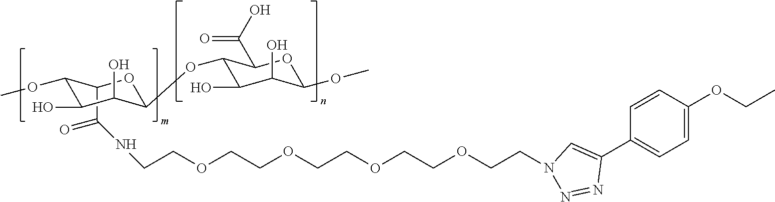 |
| Z3A43 | 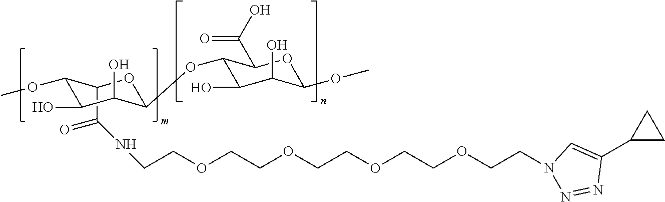 |
| B1A34 | 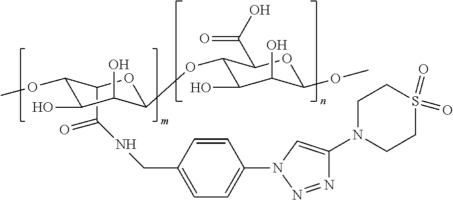 |
| B2A3 | 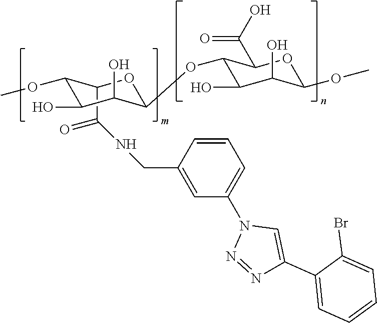 |
| B2A17 | 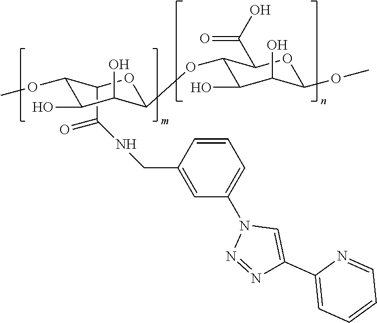 |
| B2A31 | 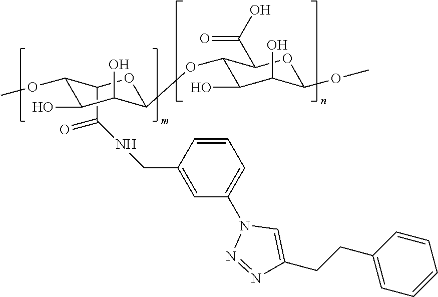 |

All the compounds of the present invention may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all the compounds of the present invention are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the compounds of the present invention have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

Compounds of the present invention may contain one or more asymmetrically substituted carbon, sulfur, or phosphorus atom and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S or the R configuration. In some embodiments, the present compounds may contain two or more atoms which have a defined stereochemical orientation.

Chemical formulas used to represent compounds of the present invention will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In some embodiments, compounds of the present invention function as prodrugs or can be derivatized to function as prodrugs. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a patient, cleaves to form a hydroxy, amino, or carboxylic acid, respectively.

In some embodiments, compounds of the present invention exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will be appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the compounds provided herein, including any solvates thereof are within the scope of the present invention.

### II. Methods of Use of Compounds

The modified alginates described herein may be used in a variety of applications in the food, pharmaceutical, cosmetic, agriculture, printing, and textile industries. Alginates are widely employed in the food industry as thickening, gelling, stabilizing, bodying, suspending, and emulsifying agents. Alternatively, these modified alginates can be used as a matrix to control the delivery of therapeutic, prophylactic, and/or diagnostic agents. Furthermore, these modified alginates can be incorporated in pharmaceutical compositions as excipients, where they can act as viscosifiers, suspension agents, emulsifiers, binders, and disintegrants. The modified alginates may also be used in other application such as a dental impression material, component of wound dressings, and as a printing agent. One of ordinary skill in the art will recognize that the modified alginates disclosed herein can be used in any application for which any currently used alginates or modified alginates may be currently employed. It is specifically contemplated that modified alginates described herein can be used in applications where improved biocompatibility and physical properties (such as being anti-fibrotic), as compared to commercially available alginates, are preferred.

i. Encapsulation of Cells

Historically, alginates and thus the modified alginates described herein may be ionically cross-linked with divalent cations, in water, at room temperature, to form a hydrogel matrix. See, for example, in U.S. Pat. No. 4,352,883. In this process, an aqueous solution containing the biological materials to be encapsulated is suspended in a solution of a water soluble polymer, the suspension is formed into droplets which are configured into discrete capsules by contact with multivalent cations, then the surface of the capsules is crosslinked with polyamino acids to form a semipermeable membrane around the encapsulated materials.

The water soluble polymer with charged side groups is crosslinked by reacting the polymer with an aqueous solution containing multivalent ions of the opposite charge, either multivalent cations if the polymer has acidic side groups or multivalent anions if the polymer has basic side groups. The cations for cross-linking of the polymers with acidic side groups to form a hydrogel are divalent and trivalent cations such as copper, calcium, aluminum, magnesium, strontium, barium, and tin, although di-, tri- or tetra-functional organic cations such as alkylammonium salts, e.g., R₃N+−−\∧∧/−−+NR₃ can also be used. Aqueous solutions of the salts of these cations are added to the polymers to form soft, highly swollen hydrogels and membranes. The higher the concentration of cation or the higher the valence, the greater the degree of cross-linking of the polymer. Concentrations from as low as 0.005 M have been demonstrated to cross-link the polymer. Higher concentrations are limited by the solubility of the salt.

The anions for cross-linking of polymers containing basic sidechains to form a hydrogel are divalent and trivalent anions such as low molecular weight dicarboxylic acids, for example, terephthalic acid, sulfate ions and carbonate ions. Aqueous solutions of the salts of these anions are added to the polymers to form soft, highly swollen hydrogels and membranes, as described with respect to cations.

A variety of polycations can be used to complex and thereby stabilize the polymer hydrogel into a semi-permeable surface membrane. Examples of materials that can be used include polymers having basic reactive groups such as amine or imine groups, having a molecular weight between 3,000 and 100,000, such as polyethylenimine and polylysine. These are commercially available. One polycation is poly(L-lysine); examples of synthetic polyamines are: polyethyleneimine, poly(vinylamine), and poly(allyl amine). There are also natural polycations such as the polysaccharide, chitosan.

Polyanions that can be used to form a semi-permeable membrane by reaction with basic surface groups on the polymer hydrogel include polymers and copolymers of acrylic acid, methacrylic acid, and other derivatives of acrylic acid, polymers with pendant SO₃H groups such as sulfonated polystyrene, and polystyrene with carboxylic acid groups.

In one embodiment, cells are encapsulated in a modified alginate polymer. In an embodiment, modified alginate capsules are fabricated from solution of modified alginate containing suspended cells using the encapsulator (such as an Inotech® encapsulator). In some embodiments, modified alginates are ionically crosslinked with a polyvalent cation, such as Ca²⁺, Ba²⁺ or Sr²⁺. In some embodiments, the modified alginate is crosslinked using BaCl₂. In some embodiments, the capsules are further purified after formation. In some embodiments, the capsules are washed with, for example, HEPES solution, Krebs solution, and/or RPMI-1640 medium.

Cells can be obtained directed from a donor, from cell culture of cells from a donor, or from established cell culture lines. In the some embodiments, cells are obtained directly from a donor, washed and implanted directly in combination with the polymeric material. The cells are cultured using techniques known to those skilled in the art of tissue culture. In the some embodiment, the cells are autologous—i.e., derived from the individual into which the cells are to be transplanted, but may be allogeneic or heterologous.

Cell attachment and viability can be assessed using scanning electron microscopy, histology, and quantitative assessment with radioisotopes. The function of the implanted cells can be determined using a combination of the above-techniques and functional assays. For example, in the case of hepatocytes, in vivo liver function studies can be performed by placing a cannula into the recipient's common bile duct. Bile can then be collected in increments. Bile pigments can be analyzed by high pressure liquid chromatography looking for underivatized tetrapyrroles or by thin layer chromatography after being converted to azodipyrroles by reaction with diazotized azodipyrroles ethylanthranilate either with or without treatment with P-glucuronidase. Diconjugated and monoconjugated bilirubin can also be determined by thin layer chromatography after alkalinemethanolysis of conjugated bile pigments. In general, as the number of functioning transplanted hepatocytes increases, the levels of conjugated bilirubin will increase. Simple liver function tests can also be done on blood samples, such as albumin production. Analogous organ function studies can be conducted using techniques known to those skilled in the art, as required to determine the extent of cell function after implantation. For example, islet cells of the pancreas may be delivered in a similar fashion to that specifically used to implant hepatocytes, to achieve glucose regulation by appropriate secretion of insulin to cure diabetes. Other endocrine tissues can also be implanted. Studies using labeled glucose as well as studies using protein assays can be performed to quantitate cell mass on the polymer scaffolds. These studies of cell mass can then be correlated with cell functional studies to determine what the appropriate cell mass is. In the case of chondrocytes, function is defined as providing appropriate structural support for the surrounding attached tissues.

This technique can be used to provide multiple cell types, including genetically altered cells, within a three-dimensional scaffolding for the efficient transfer of large number of cells and the promotion of transplant engraftment for the purpose of creating a new tissue or tissue equivalent. It can also be used for immunoprotection of cell transplants while a new tissue or tissue equivalent is growing by excluding the host immune system.

Examples of cells which can be implanted as described herein include chondrocytes and other cells that form cartilage, osteoblasts and other cells that form bone, muscle cells, fibroblasts, and organ cells. As used herein, “organ cells” includes hepatocytes, islet cells, cells of intestinal origin, cells derived from the kidney, and other cells acting primarily to synthesize and secret, or to metabolize materials. A particular cell type is a pancreatic islet cell.

The polymeric matrix can be combined with humoral factors to promote cell transplantation and engraftment. For example, the polymeric matrix can be combined with angiogenic factors, antibiotics, anti-inflammatories, growth factors, compounds which induce differentiation, and other factors which are known to those skilled in the art of cell culture.

For example, humoral factors could be mixed in a slow-release form with the cell-alginate suspension prior to formation of implant for transplantation. Alternatively, the hydrogel could be modified to bind humoral factors or signal recognition sequences prior to combination with isolated cell suspension.

The techniques described herein can be used for delivery of many different cell types to achieve different tissue structures. In one embodiment, the cells are mixed with the hydrogel solution and injected directly into a site where it is desired to implant the cells, prior to hardening of the hydrogel. However, the matrix may also be molded and implanted in one or more different areas of the body to suit a particular application. This application is particularly relevant where a specific structural design is desired or where the area into which the cells are to be implanted lacks specific structure or support to facilitate growth and proliferation of the cells.

The site, or sites, where cells are to be implanted is determined based on individual need, as is the requisite number of cells. For cells having organ function, for example, hepatocytes or islet cells, the mixture can be injected into the mesentery, subcutaneous tissue, retroperitoneum, properitoneal space, and intramuscular space. For formation of cartilage, the cells are injected into the site where cartilage formation is desired. One could also apply an external mold to shape the injected solution. Additionally, by controlling the rate of polymerization, it is possible to mold the cell-hydrogel injected implant like one would mold clay. Alternatively, the mixture can be injected into a mold, the hydrogel allowed to harden, then the material implanted.

ii. Coating Products and Surfaces

Medical products can be coated with the disclosed modified alginate polymers using a variety of techniques, examples of which include spraying, dipping, and brush coating. Polymer coatings are typically applied to the surface to be coated by dissolving a polymer in an appropriate, organic solvent, and applying by spraying, brushing, dipping, painting, or other similar technique. The coatings are deposited on the surface and associate with the surfaces via non-covalent interactions. The coated products and surfaces that result are specifically contemplated and disclosed.

In some embodiments, the surface may be pretreated with an appropriate solution or suspension to modify the properties of the surface, and thereby strengthen the non-covalent interactions between the modified surface and the coating.

The polymer solution is applied to a surface at an appropriate temperature and for a sufficient period of time to form a coating on the surface, wherein the coating is effective in forming an anti-fibrotic surface. Typical temperatures include room temperature, although higher temperatures may be used. Typical time periods include 5 minutes or less, 30 minutes or less, 60 minutes or less, and 120 minutes or less. In some embodiments the solution can be applied for 120 minutes or longer to form a coating with the desired anti-fibrotic activity. However, shorter time periods may be used. Anti-fibrotic activity can be measured in any of the ways disclosed herein or known in the art. The anti-fibrotic activity can be the foreign body response determined as described herein.

The modified alginate compounds described herein can be covalently or non-covalently associated with the products, devices, and surfaces. For those embodiments where the modified alginate compounds described herein is covalently associated with the product, device, or surface, the polymer can be attached to the product, device, or surface by, for example, functionalizing the product, device, or surface with a reaction functional group, such as a nucleophilic group, and reacting the nucleophilic group with a reaction functional group on the polymer, such as an electrophilic group. Alternatively, the polymer can be functionalized with a nucleophilic group which is reacted with an electrophilic group on the product, device, or surface.

In particular embodiments, the modified alginate compounds described herein is non-covalently associated with the product, device, or surface. The polymer can be applied to the product, device, or surface by spraying, wetting, immersing, dipping, painting, bonding or adhering or otherwise providing a product, device, or surface with a compound with the modified alginate compounds described herein. In one embodiment, the polymer is applied by spraying, painting, or dipping or immersing. For example, a polymer paint can be prepared by dissolving the modified alginate compounds described herein in a suitable solvent (generally aqueous), and optionally sonicating the solution to ensure the polymer is completely dissolved. The product, device, or surface to be coated can be immersed in the polymer solution for a suitable period of time, e.g., 5 seconds, followed by drying, such as air drying. The procedure can be repeated as many times as necessary to achieve adequate coverage. The thickness of the coating is generally from about 1 nm to about 1 cm, preferably from about 10 nm to 1 mm, more preferably from about 100 nm to about 100 microns.

The coating can be applied at the time the product, device, or surface is manufactured or can be applied subsequent to manufacture of the product, device, or surface. In some embodiments, the coating is applied to the product, device, or surface immediately prior to use of the product, device, or surface.

This is referred to an intraoperative coating. “Immediately prior”, as used herein, mean within 1, 2, 5, 10, 15, 20, 30, 45, 60, 75, 90, 120, 150, 180 minutes or greater of implantation or use. In some embodiments, the product, device, or surface is coated at the hospital, e.g., in the operating room, with 20, 15, 10, or 5 minutes of implantation or use. Coating immediately prior to use may overcome limitations of products, devices, and surfaces coated at the time of manufacture, such as damage of the coating during storage and/or transportation of the product, device, or surface and/or decrease in the efficacy of the coating over time as the coating is exposed to environmental conditions, which may be harsh (e.g., high temps, humidity, exposure to UV light, etc.).

The coated medical products can be used for the known uses and purposes of uncoated or differently coated forms of the medical products.

a. Medical Products

Medical products useful for coating include any types of medical devices used, at least in part, for implantation in the body of a patient. Examples include, but are not limited to, implants, implantable medical products, implantable devices, catheters and other tubes (including urological and biliary tubes, endotracheal tubes, wound drain tubes, needle injection catheters, preferably insertable central venous catheters, dialysis catheters, long term tunneled central venous catheters peripheral venous catheters, short term central venous catheters, arterial catheters, pulmonary catheters, Swan-Ganz catheters, urinary catheters, peritoneal catheters), vascular catheter ports, blood clot filters, urinary devices (including long term urinary devices, tissue bonding urinary devices, artificial urinary sphincters, urinary dilators), shunts (including ventricular or arterio-venous shunts, stent transplants, biliary stents, intestinal stents, bronchial stents, esophageal stents, ureteral stents, and hydrocephalus shunts), balloons, pacemakers, implantable defibrillators, orthopedic products (including pins, plates, screws, and implants), transplants (including organs, vascular transplants, vessels, aortas, heart valves, and organ replacement parts), prostheses (including breast implants, penile prostheses, vascular grafting prostheses, heart valves, artificial joints, artificial larynxes, otological implants, artificial hearts, artificial blood vessels, and artificial kidneys), aneurysm-filling coils and other coil devices, transmyocardial revascularization devices, percutaneous myocardial revascularization devices, tubes, fibers, hollow fibers, membranes, blood containers, titer plates, adsorber media, dialyzers, connecting pieces, sensors, valves, endoscopes, filters, pump chambers, scalpels, needles, scissors (and other devices used in invasive surgical, therapeutic, or diagnostic procedures), and other medical products and devices intended to have anti-fibrotic properties. The expression “medical products” is broad and refers in particular to products that come in contact with blood briefly (e.g., endoscopes) or permanently (e.g., stents).

Useful medical products are balloon catheters and endovascular prostheses, in particular stents. Stents of a conventional design have a filigree support structure composed of metallic struts. The support structure is initially provided in an unexpanded state for insertion into the body and is then widened into an expanded state at the application site. The stent can be coated before or after it is crimped onto a balloon. A wide variety of medical endoprostheses or medical products or implants for highly diverse applications and are known. They are used, for example, to support vessels, hollow organs, and ductal systems (endovascular implants), to attach and temporarily affix tissue implants and tissue transplants, and for orthopedic purposes such as pins, plates, or screws.

The modified alginate compounds described herein may be applied to, absorbed into, or coupled to, a variety of different substrates and surfaces. Examples of suitable materials include metals, metallic materials, ceramics, polymers, fibers, inert materials such as silicon, and combinations thereof.

Suitable polymeric materials include, but are not limited to, styrene and substituted styrenes, ethylene, propylene, poly(urethane)s, acrylates and methacrylates, acrylamides and methacrylamides, polyesters, polysiloxanes, polyethers, poly(orthoester), poly(carbonates), poly(hydroxyalkanoate)s, copolymers thereof, and combinations thereof.

Substrates can be in the form of, or form part of, films, particles (nanoparticles, microparticles, or millimeter diameter beads), fibers (wound dressings, bandages, gauze, tape, pads, sponges, including woven and non-woven sponges and those designed specifically for dental or ophthalmic surgeries), sensors, pacemaker leads, catheters, stents, contact lenses, bone implants (hip replacements, pins, rivets, plates, bone cement, etc.), or tissue regeneration or cell culture devices, or other medical devices used within or in contact with the body.

Implants coated with modified alginate compound coatings are described herein. “Implants” are any object intended for placement in the body of a mammal, such as a human, that is not a living tissue. Implants are a form of medical product. Implants include naturally derived objects that have been processed so that their living tissues have been devitalized. As an example, bone grafts can be processed so that their living cells are removed, but so that their shape is retained to serve as a template for ingrowth of bone from a host. As another example, naturally occurring coral can be processed to yield hydroxyapatite preparations that can be applied to the body for certain orthopedic and dental therapies. An implant can also be an article comprising artificial components. The term “implant” can be applied to the entire spectrum of medical devices intended for placement in a human body or that of a mammal, including orthopedic applications, dental applications, ear, nose, and throat (“ENT”) applications, and cardiovascular applications.

In some embodiments, “implant” as used herein refers to a macroscopic composition including a device for implantation or a surface of a device for implantation and a modified alginate compound coating. In these embodiments, the term “implant” does not encompass nanoparticles and/or microparticles. “Macroscopic” as used herein generally refers to devices, implants, or compositions that can be viewed by the unaided eye.

Examples of implantable medical devices and medical devices and mechanical structures that can use a bio-compatible coating include, but are not limited to, stents, conduits, scaffolds, cardiac valve rings, cardiovascular valves, pacemakers, hip replacement devices, implanted sensor devices, esophageal stents, heart implants, bio-compatible linings for heart valves, dialysis equipment and oxygenator tubing for heart-lung by-pass systems.

In general, a stent is a device, typically tubular in shape, that is inserted into a lumen of the body, such as a blood vessel or duct, to prevent or counteract a localized flow constriction. The purpose of a stent, in some cases, is to mechanically prop open a bodily fluid conduit. Stents are often used to alleviate diminished blood flow to organs and extremities in order to maintain adequate delivery of oxygenated blood. The most common use of stents is in coronary arteries, but they are also widely used in other bodily conduits, such as, for example, central and peripheral arteries and veins, bile ducts, the esophagus, colon, trachea, large bronchi, ureters, and urethra. Frequently, stents inserted into a lumen are capable of being expanded after insertion or are self-expanding. For example, metal stents are deployed into an occluded artery using a balloon catheter and expanded to restore blood flow. For example, stainless steel wire mesh stents are commercially available from Boston Scientific, Natick, Mass.

In some embodiments, the implant is an orthopedic implant. An “orthopedic implant” is defined as an implant which replaces bone or provides fixation to bone, replaces articulating surfaces of a joint, provides abutment for a prosthetic, or combinations thereof or assists in replacing bone or providing fixation to bone, replacing articulating surfaces of a joint, providing abutment for a prosthetic, and combinations thereof.

Orthopedic implants can be used to replace bone or provide fixation to bone, replace articulating surfaces of a joint, provide abutment for a prosthetic, or combinations thereof or assist in replacing bone or providing fixation to bone, replacing articulating surfaces of a joint, providing abutment for a prosthetic, including dental applications, and combinations thereof.

Suitable orthopedic implants include, but are not limited to, wire, Kirschner wire, bone plates, screws, pins, tacs, rods, nails, nuts, bolts, washers, spikes, buttons, wires, fracture plates, reconstruction and stabilizer devices, endo- and exoprostheses (articulating and non-articulating), intraosseous transcutaneous prostheses, spacers, mesh, implant abutments, anchors, barbs, clamps, suture, interbody fusion devices, tubes of any geometry, scaffolds, and combinations thereof.

In other embodiments, the implant is an ear, nose, and/or throat (“ENT”) implant. Exemplary ENT implants include, but are not limited to, ear tubes, endotracheal tubes, ventilation tubes, cochlear implants and bone anchored hearing devices.

In other embodiments, the implant is a cardiovascular implant. Exemplary cardiovascular implants are cardiac valves or alloplastic vessel wall supports, total artificial heart implants, ventricular assist devices, vascular grafts, stents, electrical signal carrying devices such as pacemaker and neurological leads, defibrillator leads, and the like.

Implants can be prepared from a variety of materials. In some embodiments, the material is biocompatible. In some embodiments, the material is biocompatible and non-biodegradable. Exemplary materials include metallic materials, metal oxides, polymeric materials, including degradable and non-degradable polymeric materials, ceramics, porcelains, glass, allogeneic, xenogenic bone or bone matrix; genetically engineered bone; and combinations thereof.

Suitable metallic materials include, but are not limited to, metals and alloys based on titanium (such as nitinol, nickel titanium alloys, thermo-memory alloy materials), stainless steel, tantalum, palladium, zirconium, niobium, molybdenum, nickel-chrome, or certain cobalt alloys including cobalt-chromium and cobalt-chromium-nickel alloys such as ELGILOY® and PHYNOX®.

Useful examples include stainless steel grade 316 (SS 316 L) (comprised of Fe, <0.3% C, 16-18.5% Cr, 10-14% Ni 2-3% Mo, <2% Mn, <1% Si, <0.45% P, and <0.03% S), tantalum, chromium molybdenum alloys, nickel-titanium alloys (such as nitinol) and cobalt chromium alloys (such as MP35N, ASTM Material Designation: 35Co-35Ni-20Cr-10Mo). Typical metals currently in use for stents include SS 316 L steel and MP35N. See also, “Comparing and Optimizing Co—Cr Tubing for Stent Applications,” Poncin, P, Millet, C., Chevy, J., and Profit, J. L., Materials & Processes for Medical Devices Conference, August 2004, ASM International.

Suitable ceramic materials include, but are not limited to, oxides, carbides, or nitrides of the transition elements such as titanium oxides, hafnium oxides, iridium oxides, chromium oxides, aluminum oxides, and zirconium oxides. Silicon based materials, such as silica, may also be used.

Suitable polymeric materials include, but are not limited to, polystyrene and substituted polystyrenes, polyethylene, polypropylene, polyacetylene, polystyrene, TEFLON®, poly(vinyl chloride) (PVC), polyolefin copolymers, poly(urethane)s, polyacrylates and polymethacrylates, polyacrylamides and polymethacrylamides, polyesters, polysiloxanes, polyethers, poly(orthoester), poly(carbonates), poly(hydroxyalkanoate)s, polyfluorocarbons, PEEK®, Teflon® (polytetrafluoroethylene, PTFE), silicones, epoxy resins, Kevlar®, Dacron® (a condensation polymer obtained from ethylene glycol and terephthalic acid), nylon, polyalkenes, phenolic resins, natural and synthetic elastomers, adhesives and sealants, polyolefins, polysulfones, polyacrylonitrile, biopolymers such as polysaccharides and natural latex, collagen, Cellulosic polymers (e.g., alkyl celluloses, etc.), polysaccharides, poly(glycolic acid), poly(L-lactic acid) (PLLA), a polydioxanone (PDA), or racemic poly(lactic acid), polycarbonates, (e.g., polyamides (nylon); fluoroplastics, carbon fiber, and blends or copolymers thereof.

The polymer can be covalently or non-covalently associated with the surface; however, in particular embodiments, the polymer is non-covalently associated with the surface. The polymer can be applied by a variety of techniques in the art including, but not limited to, spraying, wetting, immersing, dipping, such as dip coating (e.g., intraoperative dip coating), painting, or otherwise applying a hydrophobic, polycationic polymer to a surface of the implant.

A surface of a product adapted for use in a medical environment can be capable of sterilization using autoclaving, biocide exposure, irradiation, or gassing techniques, like ethylene oxide exposure. Surfaces found in medical environments include the inner and outer aspects of various instruments and devices, whether disposable or intended for repeated uses.

b. Hydrogels

Medical products can also be made of or using hydrogels. The modified alginate compounds described herein may form hydrogels for this and other purposes. Products made of other hydrogels can also be coated with the disclosed modified alginate polymers. Thus, the modified alginate compounds described herein may be used as a coating on a product or surface or can be used as the product itself. Hydrogels are three-dimensional, hydrophilic, polymeric networks capable of imbibing large amounts of water or biological fluids (Peppas et al. Eur. J. Pharm. Biopharm. 2000, 50, 27-46). These networks are composed of homopolymers or copolymers, and are insoluble due to the presence of chemical crosslinks or physical crosslinks, such as entanglements or crystallites. Hydrogels can be classified as neutral or ionic, based in the nature of the side groups. In addition, they can be amorphous, semicrystalline, hydrogen-bonded structures, supermolecular structures and hydrocolloidal aggregates (Peppas, N. A. Hydrogels. In: Biomaterials science: an introduction to materials in medicine; Ratner, B. D., Hoffman, A. S., Schoen, F. J., Lemons, J. E., Eds; Academic Press, 1996, pp. 60-64; Peppas et al., Eur. J. Pharm. Biopharm. 2000, 50, 27-46). Hydrogels can be prepared from synthetic or natural monomers or polymers. The hydrogels may include the modified alginate compounds described herein.

Hydrogels can be prepared from synthetic polymers such as poly(acrylic acid) and its derivatives [e.g. poly(hydroxyethyl methacrylate) (pHEMA)], poly(N-isopropylacrylamide), poly(ethylene glycol) (PEG) and its copolymers and poly(vinyl alcohol) (PVA), among others (Bell and Peppas, Adv. Polym. Sci. 122:125-175 (1995); Peppas et al., Eur. J. Pharm. Biopharm. 50:27-46 (200); Lee and Mooney, Chem. Rev. 101:1869-1879 (2001)). Hydrogels prepared from synthetic polymers are in general non-degradable in physiologic conditions. Hydrogels can also be prepared from natural polymers including, but not limited to, polysaccharides, proteins, and peptides. The modified alginate compounds described herein are one example. These networks are in general degraded in physiological conditions by chemical or enzymatic means.

In some embodiments, the hydrogel is non-degradable under relevant in vitro and in vivo conditions. Stable hydrogel coatings are necessary for certain applications including central venous catheters coating, heart valves, pacemakers and stents coatings. In other cases, hydrogel degradation may be a preferential approach such as in tissue engineering constructs.

In some embodiments, the hydrogel can be formed by dextran. Dextran is a bacterial polysaccharide, consisting essentially of α-1,6 linked D-glucopyranose residues with a few percent of α-1,2, α-1,3, or α-1,4-linked side chains, Dextran is widely used for biomedical applications due to its biocompatibility, low toxicity, relatively low cost, and simple modification. This polysaccharide has been used clinically for more than five decades as a plasma volume expander, peripheral flow promoter and antithrombolytic agent (Mehvar, R. J. Control. Release 2000, 69, 1-25). Furthermore, it has been used as macromolecular carrier for delivery of drugs and proteins, primarily to increase the longevity of therapeutic agents in the circulation. Dextran can be modified with vinyl groups either by using chemical or enzymatic means to prepare gels (Ferreira et al. Biomaterials 2002, 23, 3957-3967).

Dextran-based hydrogels prevent the adhesion of vascular endothelial, smooth muscle cells, and fibroblasts (Massia, S. P.; Stark, J. J. Biomed Mater. Res. 2001, 56, 390-399. Ferreira et al. 2004, J. Biomed. Mater. Res. 68, 584-596) and dextran surfaces prevent protein adsorption (Osterberg et al. J. Biomed. Mat. Res. 1995, 29, 741-747).

As described herein, the modified alginate compounds described herein can be used to encapsulate cells. In some embodiments, the encapsulated cells can be fabricated into a macrodevice. For example, in some embodiments, cells encapsulated in modified alginate hydrogel can be coated onto a surface, such as a planar surface. In some embodiments, capsules containing cells can be adhered to tissue of a subject using a biocompatible adhesive. In other embodiments, capsules containing cells can be coated onto a medical device suitable for implantation.

iii. Treating Diseases or Disorders

Alternatively, the encapsulated cells can be transplanted into a patient in need thereof to treat a disease or disorder. In some embodiments, the encapsulated cells are obtained from a genetically non-identical member of the same species. In alternative embodiments, the encapsulated cells are obtained from a different species than the patient. In some embodiments, hormone- or protein-secreting cells are encapsulated and transplanted into a patient to treat a disease or disorder.

In some embodiments, the disease or disorder is caused by or involves the malfunction hormone- or protein-secreting cells in a patient. In a some embodiment, the disease or disorder is diabetes. Medical products, devices, and surfaces coated with a modified alginate compounds described herein can be transplanted or implanted into a patient in need thereof to treat a disease or disorder. The disclosed capsules, products, devices, and surfaces can remain substantially free of fibrotic effects, or can continue to exhibit a reduced foreign body response, for 2 weeks, 3 weeks, 4 weeks, 5 weeks, 6 weeks, 7 weeks, 8 weeks, 9 weeks, 10 weeks, 2 months, 3 months, 4 months, 5 months, 6 months, 7 months, 8, months, 9 months, 10 months, 11 months, 1 year, 2 years, or longer after administration or implantation.

The disclosed capsules, products, devices, and surfaces can be administered or implanted alone or in combination with any suitable drug or other therapy. Such drugs and therapies can also be separately administered (i.e., used in parallel) during the time the capsules, products, devices, and surfaces are present in a patient. Although the disclosed capsules, products, devices, and surfaces reduce fibrosis and immune reaction to the capsules, products, devices, and surfaces, use of anti-inflammatory and immune system suppressing drugs together with or in parallel with the capsules, products, devices, and surfaces is not excluded. In one embodiment, however, the disclosed capsules, products, devices, and surfaces are used without the use of anti-inflammatory and immune system suppressing drugs. In some embodiments, fibrosis remains reduced after the use, concentration, effect, or a combination thereof, of any anti-inflammatory or immune system suppressing drug that is used falls below an effective level. For example, fibrosis can remain reduced for 2 weeks, 3 weeks, 4 weeks, 5 weeks, 6 weeks, 7 weeks, 8 weeks, 9 weeks, 10 weeks, 2 months, 3 months, 4 months, 5 months, 6 months, 7 months, 8, months, 9 months, 10 months, 11 months, 1 year, 2 years, or longer after the use, concentration, effect, or a combination thereof, of any anti-inflammatory or immune system suppressing drug that is used falls below an effective level.

### III. Combination Therapy

In addition to being used as a monotherapy, the compounds of the present invention may also find use in combination with one or more other therapies. Effective combination therapy may be achieved with a single composition or pharmacological formulation that includes both agents, or with two distinct compositions or formulations, administered at the same time, wherein one composition includes a compound of this invention, and the other includes the second agent(s). Alternatively, the therapy may precede or follow the other agent treatment by intervals ranging from minutes to months.

Non-limiting examples of such combination therapy include combination of one or more compounds of the invention with another anti-inflammatory agent, a chemotherapeutic agent, radiation therapy, an antidepressant, an antipsychotic agent, an anticonvulsant, a mood stabilizer, an anti-infective agent, an antihypertensive agent, a cholesterol-lowering agent or other modulator of blood lipids, an agent for promoting weight loss, an antithrombotic agent, an agent for treating or preventing cardiovascular events such as myocardial infarction or stroke, an antidiabetic agent, an agent for reducing transplant rejection or graft-versus-host disease, an anti-arthritic agent, an analgesic agent, an anti-asthmatic agent or other treatment for respiratory diseases, or an agent for treatment or prevention of skin disorders.

### IV. Definitions

The definitions below supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanyl” means —N═C═O; “azido” means —N₃; in a monovalent context “phosphate” means —OP(O)(OH)₂ or a deprotonated form thereof, in a divalent context “phosphate” means —OP(O)(OH)O— or a deprotonated form thereof; “mercapto” means —SH; and “thio” means ═S; “thiocarbonyl” means —C(═S)—; “sulfonyl” means —S(O)₂—; and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, the formula

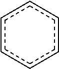

covers, for example,

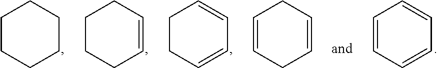

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

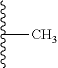

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page.” The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

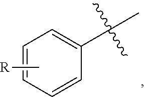

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

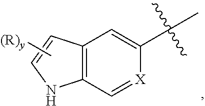

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “alkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C₁₋₄-alkyl”, “C1-4-alkyl”, “alkyl_{(C1-4)}”, and “alkyl_{(C≤4)}” are all synonymous. Except as noted below, every carbon atom is counted to determine whether the group or compound falls with the specified number of carbon atoms. For example, the group dihexylamino is an example of a dialkylamino_{(C12)} group; however, it is not an example of a dialkylamino_{(C6)} group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic R system. An aromatic compound or chemical group may be depicted as a single resonance structure; however, depiction of one resonance structure is taken to also refer to any other resonance structure. For example:

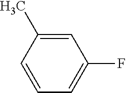

is also taken to refer to

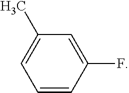

Aromatic compounds may also be depicted using a circle to represent the delocalized nature of the electrons in the fully conjugated cyclic π system, two non-limiting examples of which are shown below;

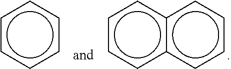

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H-R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

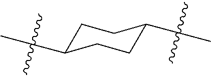 [SMILES: *C1CCC(*)CC1]

is a non-limiting example of cycloalkanediyl group. A “cycloalkane” refers to the class of compounds having the formula H-R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H-R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “alkynyl” refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H-R, wherein R is alkynyl.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

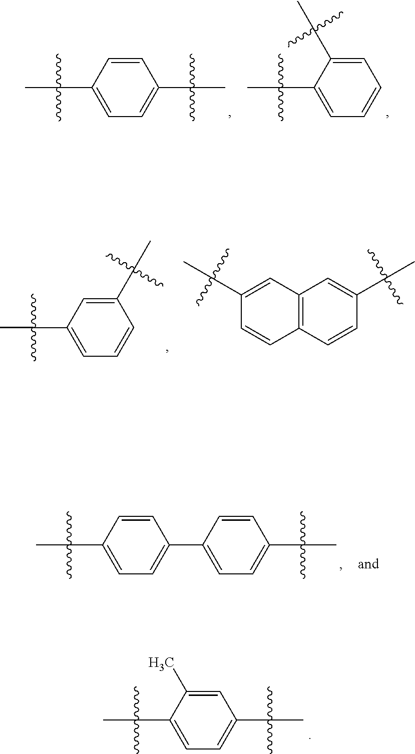

An “arene” refers to the class of compounds having the formula H-R, wherein R is aryl as that term is defined above. Benzene and toluene are non-limiting examples of arenes.

The term “aralkyl” refers to the monovalent group-alkanediyl-aryl, in which the terms alkanediyl and aryl are each used in a manner consistent with the definitions provided above. Non-limiting examples are: phenylmethyl (benzyl, Bn) and 2-phenyl-ethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl, isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H-R, wherein R is heteroaryl. Pyridine and quinoline are non-limiting examples of heteroarenes. The term “heteroarenediyl” refers to a divalent aromatic group, with two aromatic carbon atoms, two aromatic nitrogen atoms, or one aromatic carbon atom and one aromatic nitrogen atom as the two points of attachment, said atoms forming part of one or more aromatic ring structures, each with three to eight ring atoms, wherein at least one of the ring atoms of the aromatic ring structure(s) is nitrogen, oxygen or sulfur, and wherein the divalent group consists of no atoms other than carbon, hydrogen, aromatic nitrogen, aromatic oxygen and aromatic sulfur. If more than one ring is present, the rings are fused; however, the term heteroarenediyl does not preclude the presence of one or more alkyl or aryl groups (carbon number limitation permitting) attached to one or more ring atoms. Non-limiting examples of heteroarenediyl groups include:

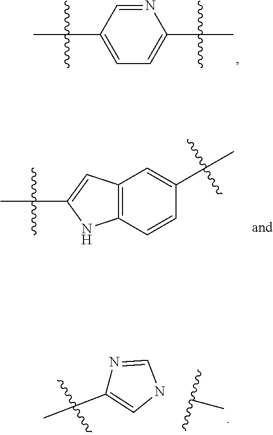

The term “heteroaralkyl” refers to the monovalent group-alkanediyl-heteroaryl, in which the terms alkanediyl and heteroaryl are each used in a manner consistent with the definitions provided above. Non-limiting examples are: pyridinylmethyl and 2-quinolinyl-ethyl.

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings are fused. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. Non-limiting examples of N-heterocycloalkyl groups include N-pyrrolidinyl and

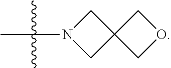 [SMILES: *N1CC2(COC2)C1]

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group.

The term “alkoxy” refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The term “amido” (acylamino), when used without the “substituted” modifier, refers to the group —NHR, in which R is acyl, as that term is defined above. A non-limiting example of an amido group is —NHC(O)CH₃.

When a chemical group is used with the “substituted” modifier, one or more hydrogen atom(s) of the group has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “hydroxyalkyl” is a subset of substituted alkyl, in which one or more hydrogen atom has been replaced with a hydroxy (i.e. —OH) group, such that no other atoms aside from carbon, hydrogen, and oxygen are present. The groups —CH₂OH, —CH₂CH₂OH, —CH(OH)CHOH, —CH₂CH(OH)CH₃, and —CH(OH)CH₂OH are non-limiting examples of hydroxyalkyl groups. The term “monohydroxyalkyl” is a subset of substituted alkyl, in which one hydrogen atom has been replaced with a hydroxy (i.e. —OH) group, such that no other atoms aside from carbon, hydrogen, and one oxygen are present. The groups —CH₂OH, —CH₂CH₂OH, and —CH₂CH(OH)CH₃ are non-limiting examples of monohydroxyalkyl groups. The term “fluoroalkyl” is a subset of substituted alkyl, in which one or more hydrogen atom has been replaced with a fluoro, such that no other atoms aside from carbon, hydrogen, and fluorine are present. The groups —CH₂F, —CHF₂, and —CF₃ are non-limiting examples of fluoroalkyl groups. The term “monofluoroalkyl” is a subset of substituted alkyl, in which one hydrogen atom has been replaced with a fluoro, such that no other atoms aside from carbon, hydrogen, and one fluorine are present. The groups —CH₂F, —CH₂CH₂F, and —CH₂CH(F)CH₃ are non-limiting examples of monofluoroalkyl groups. The term “aminoalkyl” is a subset of substituted alkyl, in which one or more hydrogen atom has been replaced with an amino (i.e. —NH₂) group, such that no other atoms aside from carbon, hydrogen, and nitrogen are present. The groups —CH₂NH₂, —CH(NH₂)CH₃, —CH₂CH₂NH₂, —CH₂CH(NH₂)CH₃ and —CH(NH₂)CH₂NH₂ are non-limiting examples of aminoalkyl groups. The term “monoaminoalkyl” is a subset of substituted alkyl, in which one hydrogen atom has been replaced with an amino (i.e. —NH₂) group, such that no other atoms aside from carbon, hydrogen, and one nitrogen are present. The groups —CH₂NH₂, —CH₂CH₂NH₂, and —CH₂CH(NH₂)CH₃ are non-limiting examples of monoaminoalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients.

An “active ingredient” (AI) or active pharmaceutical ingredient (API) (also referred to as an active compound, active substance, active agent, pharmaceutical agent, agent, biologically active molecule, or a therapeutic compound) is the ingredient in a pharmaceutical drug that is biologically active.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to the patient or subject, is sufficient to effect such treatment or prevention of the disease as those terms are defined below.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an active pharmaceutical ingredient of the present invention. The prodrug itself may or may not have activity in its prodrug form. For example, a compound comprising a hydroxy group may be administered as an ester that is converted by hydrolysis in vivo to the hydroxy compound. Non-limiting examples of suitable esters that may be converted in vivo into hydroxy compounds include acetates, citrates, lactates, phosphates, tartrates, malonates, oxalates, salicylates, propionates, succinates, fumarates, maleates, methylene-bis-p-hydroxynaphthoate, gentisates, isethionates, di-p-toluoyltartrates, methanesulfonates, ethanesulfonates, benzenesulfonates, p-toluenesulfonates, cyclohexylsulfamates, quinates, and esters of amino acids. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Experimental Procedures and Characterization Data

### A. Novel Biomaterial Generation Library Based on Alginate Derivatives

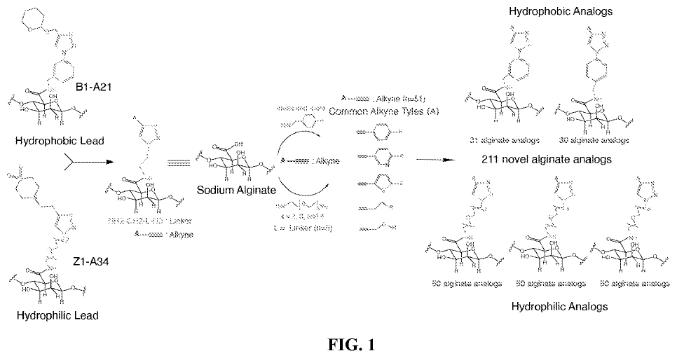

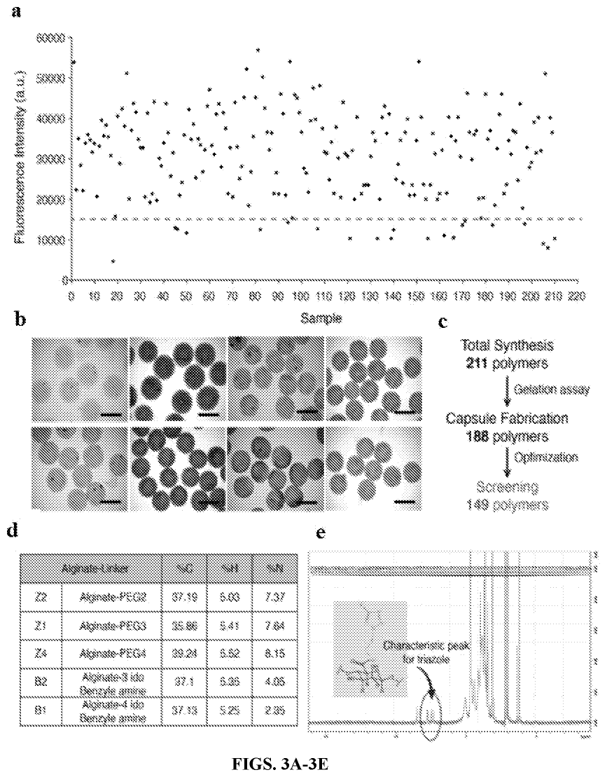

Combinatorial libraries of hydrogels were developed to identify materials with reduced recognition in preclinical fibrosis models using C57BL/6 mice using genetic barcoding techniques. Although the physicochemical parameters governing anti-fibrotic properties are not fully understood at this time, a combinatorial biomaterial screening approach was developed to generate a library of alginate-based hydrogels, utilizing several reverse chemical reactions that covalently modify latent functionalities and properties on a polymeric alginate backbone. This synthetic chemical scheme enables rapid production of a diverse series of chemical structures that are stereospecific, and the reaction is compatible with alginate compound modification. Low molecular weight, ultrapure VLVG alginate (Nova Matrix inc.) with high guluronate content was used as the starting material and synthesis of a 6872-member alginate analog library with a variety of amines, alcohols, azides, and alkynes is proposed. In the initial phase, a total of 211 alginate analogues were synthesized by maintaining the triazole ring throughout. Among the 211 unique alginate analog polymers, ˜150 analogs were generated from the combinatorial synthesis of three hydrophilic PEG-based linkers resembling the reported hydrophilic lead (Z1-A34) with 51 new alkynes, and 61 triazole containing analogs from two hydrophobic linkers resembling the hydrophobic lead (B1-A21) (FIG. 1, Table 2). The covalently attached triazole containing alginate analogs were characterized using elemental analysis and nuclear magnetic resonance spectroscopy (NMR) (FIG. 3D, 3E). Following confirmation of synthesis, purity, solubility, and gel-forming ability (gelation assay), 149 alginate analogs were selected for in vivo screening purposes (FIG. 3). Elemental analysis has shown that up to 20-30% of the alginate can be modified using the optimized reaction conditions, providing for significant modulation of material properties.

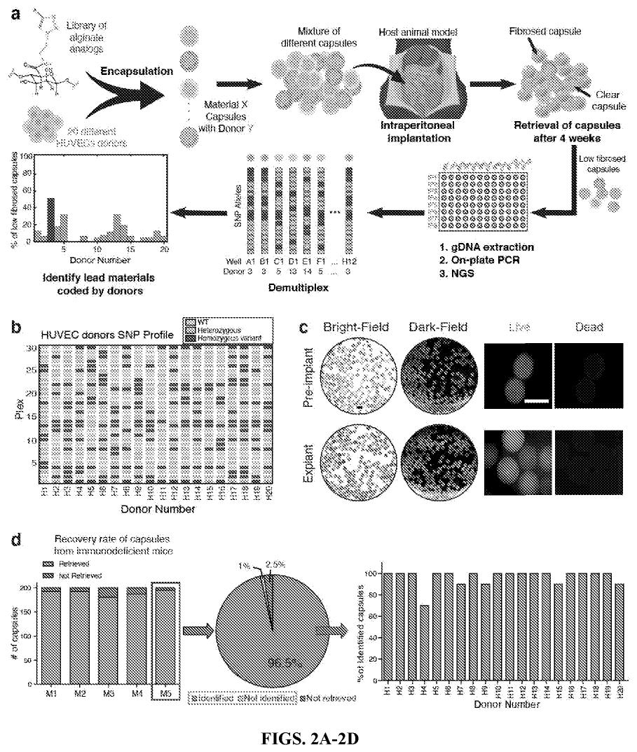

A novel high throughput in vivo screening method was developed both in rodents models that utilizes xenogeneic transplantation of human cells into profibrotic C57BL/6 model rodents for testing multiple biomaterials at a single implantation site using high-throughput biomaterial barcoding and analysis. These methods comprise tagging each biomaterial with a barcode cell. A bar-coding technique was developed using 20 different unique HUVECs, which was employed to in vivo screen a large library of immunoprotective chemically modified triazole containing hydrogels biomaterials in mouse and NHP model using next generation sequencing (NGS). See FIG. 2A.

TABLE 2
Table of linkers and alkynes employed with notations.
Alkyne Table

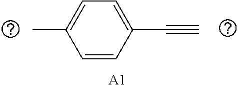

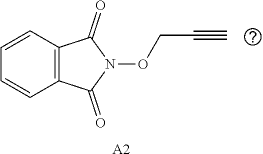

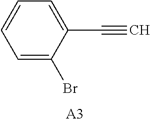

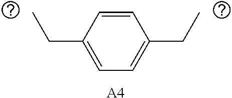

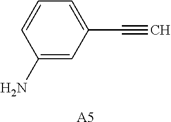

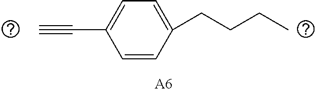

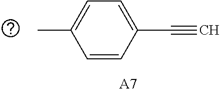

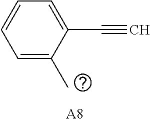

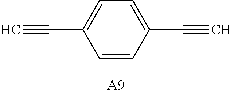

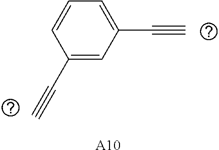

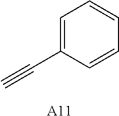

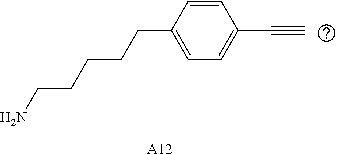

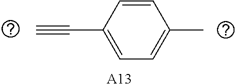

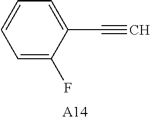

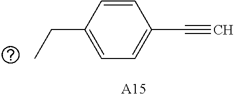

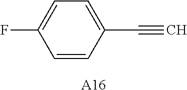

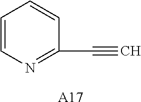

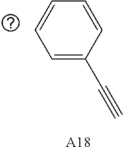

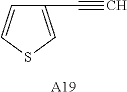

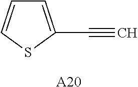

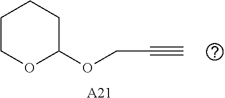

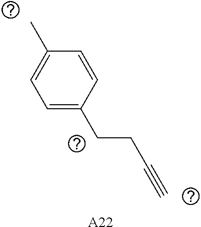

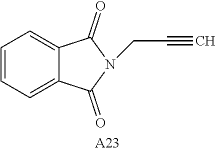

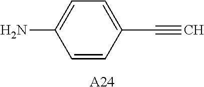

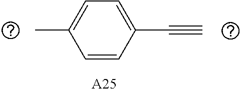

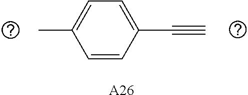

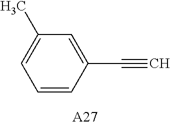

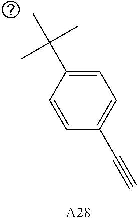

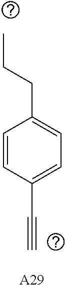

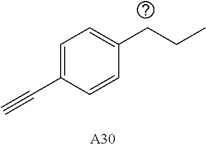

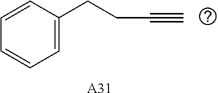

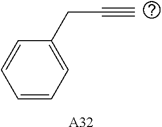

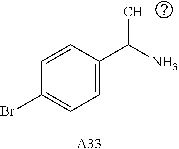

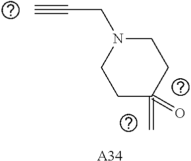

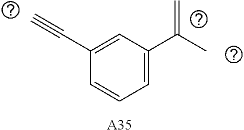

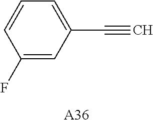

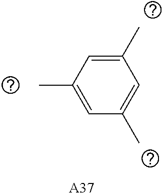

[symbol]

A38

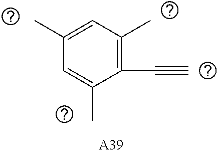

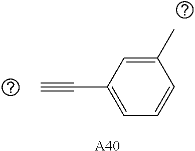

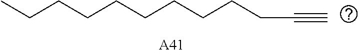

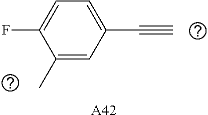

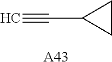

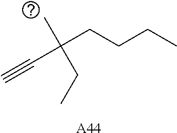

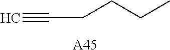

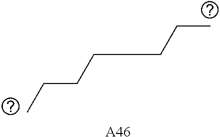

[symbol]

A47

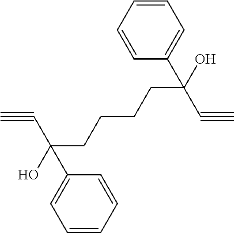 [SMILES: C#CC(O)(CCCCC(O)(C#C)c1ccccc1)c1ccccc1]

A48

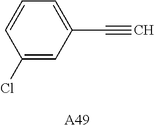

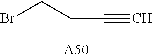

Linker Table

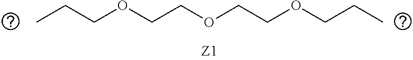

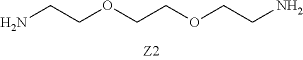

[symbol]

Z4

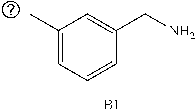

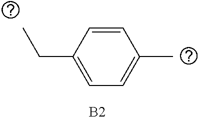

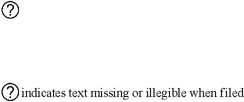

[symbol]

indicates data missing or illegible when filed

TABLE 3
Information for the 20 different HUVEC donors

| Donor Code | Cells | Company | Cat No. | Lot No. |
|---|---|---|---|---|
| H1 | Human | Lonza | CC-2517 | 0000260634 |
| H2 | Umbilical Vein | | | 0000315132 |
| H3 | Endothelial | | | 0000315288 |
| H4 | Cells | | | 0000317156 |
| H5 | (HUVECs) | | | 0000317687 |
| H6 | | | | 18TL142763 |
| H7 | | | | 0000321046 |
| H8 | | | | 0000319181 |
| H9 | | | | 0000319180 |
| H10 | | | | 0000318930 |
| H11 | | | | 0000316669 |
| H12 | | | | 0000198228 |
| H13 | | | | 0000254439 |
| H14 | | | | 0000246600 |
| H15 | | | | 0000246083 |
| H16 | | | | 0000278143 |
| H17 | | | | 0000273864 |
| H18 | | | | 0000273863 |
| H19 | | | | 0000269280 |
| H20 | | | | 0000269279 |

TABLE 4
Table of synthesized alginate analogues
and screened in vivo (mice and NHP).
Synthesized with this linker (YS/NS)-Screened in Mice (YM/NM)

| Alkyne | Z1 | Z2 | Z4 | B1 | B2 |
|---|---|---|---|---|---|
| A1 | YS-YM | YS-NM | YS-YM | YS-YM | YS-YM |
| A2 | YS-YM | YS-YM | YS-YM | YS-NM | YS-YM |
| A3 | YS-YM | YS-YM | YS-YM | NS-NM | YS-YM |
| A4 | YS-YM | YS-YM | YS-YM | NS-NM | YS-YM |
| A5 | YS-YM | NS-NM | YS-NM | YS-YM | YS-NM |
| A6 | YS-NM | NS-NM | YS-YM | NS-NM | YS-YM |
| A7 | YS-YM | YS-YM | YS-YM | NS-NM | YS-YM |
| A8 | YS-YM | YS-YM | YS-YM | NS-NM | YS-YM |
| A9 | YS-YM | NS-NM | YS-YM | YS-NM | YS-YM |
| A10 | YS-YM | YS-YM | YS-YM | YS-YM | YS-YM |
| A11 | YS-NM | NS-NM | YS-YM | NS-NM | YS-YM |
| A12 | YS-NM | NS-NM | YS-YM | YS-YM | YS-YM |
| A13 | YS-YM | YS-YM | YS-YM | NS-NM | YS-YM |
| A14 | YS-YM | YS-YM | YS-YM | YS-NM | YS-YM |
| A15 | YS-YM | YS-YM | YS-YM | YS-YM | YS-YM |
| A16 | YS-YM | YS-YM | YS-YM | NS-NM | YS-YM |
| A17 | YS-YM | YS-NM | YS-YM | NS-NM | YS-YM |
| A18 | YS-YM | YS-YM | YS-YM | NS-NM | YS-YM |
| A19 | YS-YM | YS-YM | YS-YM | NS-NM | YS-YM |
| A20 | NS-NM | YS-YM | YS-YM | NS-NM | YS-YM |
| A21 | YS-YM | YS-YM | YS-YM | NS-NM | YS-YM |
| A22 | YS-NM | YS-YM | YS-YM | NS-NM | YS-YM |
| A23 | YS-YM | YS-YM | YS-YM | YS-YM | YS-YM |
| A24 | YS-YM | NS-NM | YS-YM | YS-NM | YS-NM |
| A25 | YS-YM | YS-YM | YS-YM | NS-NM | YS-YM |
| A26 | YS-YM | YS-YM | YS-YM | YS-YM | YS-YM |
| A27 | YS-YM | YS-YM | YS-YM | YS-NM | YS-YP |
| A28 | YS-YM | YS-YM | YS-YM | NS-NM | YS-YM |
| A29 | YS-YM | YS-YM | YS-YM | NS-NM | YS-YM |
| A30 | YS-YM | YS-YM | YS-YM | NS-NM | YS-YM |
| A31 | YS-YM | YS-YM | YS-YM | YS-YM | YS-YM |
| A32 | YS-YM | YS-YM | YS-YM | YS-YM | YS-YM |
| A33 | YS-NM | YS-YM | YS-YM | YS-YM | YS-YM |
| A34 | YS-NM | YS-NM | YS-YM | YS-YM | YS-YM |
| A35 | YS-NM | YS-YM | YS-YM | YS-NM | YS-NM |
| A36 | YS-NM | YS-YM | YS-YM | YS-YM | NS-NM |
| A37 | YS-YM | YS-YM | NS-NM | NS-NM | NS-NM |
| A38 | YS-YM | YS-YM | NS-NM | YS-YM | NS-NM |
| A39 | YS-YM | NS-NM | NS-NM | NS-NM | NS-NM |
| A40 | YS-YM | YS-YM | NS-NM | NS-NM | NS-NM |
| A41 | YS-NP | NS-NM | NS-NM | NS-NM | NS-NM |
| A42 | YS-YM | YS-NM | YS-YM | NS-NM | NS-NM |
| A43 | YS-YM | YS-NM | YS-YM | NS-NM | NS-NM |
| A44 | YS-YM | YS-NM | YS-YM | NS-NM | NS-NM |
| A45 | YS-NM | NS-NM | YS-NM | NS-NM | NS-NM |
| A46 | YS-YM | NS-NM | YS-YM | NS-NM | NS-NM |
| A47 | YS-YM | NS-NM | YS-YM | NS-NM | NS-NM |
| A48 | NS-NM | NS-NM | NS-NM | NS-NM | NS-NM |
| A49 | YS-YM | NS-NM | NS-NM | NS-NM | NS-NM |
| A50 | YS-NM | NS-NM | NS-NM | NS-NM | NS-NM |

### B. Material and Methods

Reagents. All chemicals were from Sigma Aldrich (unless mentioned otherwise), USA and used without any further purification. Alginates used in this study are described in Table 5.

TABLE 5
Table of alginate formulations.

| Name | Company | Batch no. | Viscosity [mPa*s] | Usage |
|---|---|---|---|---|
| UP-VLVG | Novamatrix | BP-1903-04 | <20 | Starting material for |
| | | | | alginate modification |
| SLG20 | | BP-1811-27 | 20-200 | Control |
| | | | | (Unmodified alginate) |
| SLG100 | | BP-1806-13 | 20-200 | Blending material with |
| | | | | modified alginate |

High throughput modified alginate analog synthesis. To generate high-resolution structure-function relationships, a library of 211 next generation analogs (containing the crucial triazole ring) was synthesized that incorporate additional structural changes, such as chain extensions, and differential substitution.

Generic strategy for the synthesis and characterization of alginate. Amide coupling reaction were performed using one equivalent of UPVLVG alginate and one equivalent of amine linkers (5 different amine linkers in Table 2) in the presence of coupling agent of 0.5 equivalent of 4-(4,6-dimethoxy-1,3,5-triazin-2-yl)-4-methylmorpholinium chloride resulting in 5 distinct amine linker conjugated alginate polymers. These linker-modified alginate derivatives were purified by dialysis for 3 days (in saline and water) using a 10-12 kDa dialysis membrane followed by lyophilization. These 5 molecules were characterized by NMR and elemental analysis for their purity and % modification of the starting alginates. In the following step, one equivalent of alkynes (45: alkynes) were conjugated with the appropriately modified alginates by copper-catalyzed click reactions. A total of 211 numbers of triazole containing alginate derivatives were generated from the synthesis. All 211 different alginates were purified by dialysis followed by lyophilization and chemically characterized by NMR.

### Optimized Syntheses for Preparation of Small Molecules.

Z2-A19 amine: 3-ethynylthiophene (1 equiv., 4 g, 36.98 mmol) was added in a 1 L round bottom flask containing 420 mL of 5:1 methanol:water (350 mL methanol and 70 mL water) (5:1 methanol:water) followed by addition of tris((1-benzyl-4-triazolyl)methyl)amine (0.25 equiv., 2.932 gm, 5.52 mmol)) and stirring for 15 minutes. This was followed by addition of triethylamine (0.25 equiv., 0.77 mL, 5.52 mmol) and copper iodide (0.1 equiv., 422 mg, 2.22 mmol). The reaction flask was cooled to 0° C. over 15 minutes while being purged with argon before adding 11-azido-peg-2-amine (1 equiv., 5.86 mL, 36.98 mmol). The reaction was stirred for 5 mins at room temperature followed by overnight stirring at 55° C. The reaction mixture was filtered over Celite® and the solvent was removed using rotavap. The crude reaction was then purified by liquid chromatography with dichloromethane:ultra (22% MeOH in DCM with 3% NH₄OH) mixture 0% to 40% on a 120 gm ISCO silica column.

Z1-A3 amine: 1-bromo-2-ethynylbenzene (1 equiv., 2 g, 11 mmol) was added in a 250 mL round bottom flask containing 180 mL of 5:1 methanol:water (150 mL methanol and 30 mL water) (5:1 methanol:water) followed by dropwise addition of tris((1-benzyl-4-triazolyl)methyl)amine (0.25 equiv., 1.466 gm, 2.76 mmol) dissolved in 24 mL of 5:1 methanol:water (20 mL methanol and 4 mL water) and stirring for 15 minutes. This was followed by addition of triethylamine (0.25 equiv., 0.385 mL, 2.76 mmol) and copper iodide (0.1 equiv., 211 mg, 1.11 mmol). The reaction flask was cooled to 0° C. over 15 minutes while being purged with argon before adding 11-azido-peg-3-amine (1 equiv., 2.193 mL, 11.05 mmol). The reaction was stirred for 5 mins at room temperature followed by overnight stirring at 55° C. The reaction mixture was filtered over Celite® and the solvent was removed using rotavap. The crude reaction was then purified by liquid chromatography with dichloromethane:ultra (22% MeOH in DCM with 3% NH₄OH) mixture 0% to 40% on a 120 gm ISCO silica column.

Synthesis of Z4-A10 amine: 1,3-diethynylbenzene (1 equiv., 4 g, 32 mmol) was added in a 1000 mL round bottom flask containing 450 mL of 5:1 methanol:water (375 mL methanol and 75 mL water) followed by dropwise addition of tris((1-benzyl-4-triazolyl)methyl)amine (0.25 equiv., 2.932 gm, 5.52 mmol) dissolved in 30 mL of 5:1 methanol:water (25 mL methanol and 5 mL water) and stirring for 15 minutes. This was followed by addition of triethylamine (0.25 equiv., 0.77 mL, 5.52 mmol) and copper iodide (0.1 equiv., 422 mg, 2.22 mmol). The reaction flask was cooled to 0° C. for 15 minutes while being purged with argon before adding 11-azido-peg-4-amine (1 equiv., 6.92 mL, 32 mmol). The reaction was stirred for 5 mins at room temperature followed by overnight stirring at 55° C. The reaction mixture was filtered over Celite®, and the solvent was removed using rotavap. The crude reaction was then purified by liquid chromatography with dichloromethane:ultra (22% MeOH in DCM with 3% NH₄OH) mixture 0% to 40% on a 120 gm ISCO silica column.

Synthesis of B2-A17 amine: 3-iodobenzylamine (1 equiv., 4 g, 14.8 mmol) was added in a 50 mL round bottom flask containing 24 mL of methanol followed by sequential addition of triethylamine (2.4 equiv., 3.6 gm, 35.62 mmol), sodium azide (2 equiv., 1.93 g, 29.68 mmol), water (6 mL), copper iodide (0.15 equiv., 423.89 mg, 2.22 mmol), sodium ascorbate (0.1 equiv., 293.95, 0.1 eq, 2.97 mmol), 1.83 ml trans-N-N′-dimethylcyclohexene-1,2-diamine (0.2 equiv., 422.12 mg, 2.97 mmol). The mixture was evacuated and flashed with argon three times, and (4-(4-(pyridine-2-yl)-1H-1,2,3-triazol-1-yl)phenyl)methanamine (1 equiv., 3.72 g, 14.8 mmol) was added. The reaction was stirred for 5 mins at room temperature followed by overnight stirring at 55° C. The reaction mixture was filtered over Celite®, and the solvent was removed using rotavap. The crude reaction was then purified by liquid chromatography with dichloromethane:ultra (22% MeOH in DCM with 3% NH₄OH) mixture 0% to 40% on a 120 gm ISCO silica column.

Synthesis of B1-A51 amine: (4-iodophenyl)methanamine (1 equiv., 2 g, 15.13 mmol) was added to 90 mL of methanol. Triethylamine (2.4 equiv., 3.67 g, 36.3 mmol) and sodium azide (2 equiv., 1.97 g, 30.3 mmol) were added to the reaction flask. 40 mL of ultrapure water was added to the flask after everything previously added was dissolved. Sodium ascorbate (0.1 equiv., 300 mg, 1.5 mmol) and copper iodide (0.15 equiv., 432 mg, 2.27 mmol) were added to the flask. The flask was argon purged by bubbling argon through the mixture for 15 minutes. Trans-N,N′-dimethylcyclohexane-1,2-diamine (0.2 equiv., 430 mg, 3.03 mmol) and 1-ethynyl-2-methoxybenzene (1 equiv., 2 g, 15.13 mmol) were added to the flask. The reaction was stirred for 5 mins at room temperature followed by overnight stirring at 55° C. under argon. The reaction mixture was filtered over Celite®, and the solvent was removed using rotavap. The crude reaction was then purified by liquid chromatography with dichloromethane:ultra (22% MeOH in DCM with 3% NH₄OH) mixture 0% to 40% on a 120 gm ISCO silica column.

Synthesis of Z1-A34 amine: 4-Propargylthiomorpholine 1,1-Dioxide (1 eq.) was added to a 250 mL round bottom flask and dissolved in methanol:water mixture (5:1). Consequently, Tris[(1-benzyl-1H-1,2,3-triazol-4-yl)methyl]amine (0.25 eq.), Triethylamine (0.25 eq.), and copper iodide (0.1 eq.) was added. The reaction mixture was purged with argon for 15 mins and cooled to 0° C., after which 11-azido-3,6,9-trioxaundecan-1-amine (1 eq., 6.30 g, 28.86 mmol) was added. The reaction mixture was stirred at room temperature for 15 mins and afterward heated to 55° C. for overnight. The reactions were cooled to room temperature and filtered through Celite® to remove any insoluble parts. The filtrate was dried using rotavap under reduced pressure with silica. The crude reaction was then purified by liquid chromatography with dichloromethane:ultra (22% MeOH in DCM with 3% NH₄OH) mixture 0% to 40% on a 120 gm ISCO silica column and further characterized with ESI mass and NMR mass spectroscopy.

Alginate reaction: 1.5 g of VLVG (1 equiv) was dissolved in 45 ml of water. Then 7.65 mmol of the Z2-A19, Z1-A3 (1 eq) amine was dissolved in 22.5 ml acetonitrile and added to the mixture. Following it, an aqueous solution of 4-(4,6-Dimethoxy-1,3,5-triazin-2-yl)-4-methylmorpholinium chloride (0.75 eq) was added dropwise to the mixture. The reaction was stirred overnight at 55° C. The solvent was removed under reduced pressure and the solid was dissolved in water. The solution was filtered through a pad of cyano-functionalized silica and the water was removed under reduced pressure to concentrate the solution. It was then dialyzed against a 10,000 MWCO membrane in DI water for three days. The water was removed under reduced pressure and lyophilized to obtain functionalized alginates.

Synthesis of methacryloyl Z1A3 (Met-Z1A3): 2-(2-(2-(2-(4-(2-bromophenyl)-1H-1,2,3-triazol-1-yl)ethoxy)ethoxy)ethoxy)ethan-1-amine (1 equiv., 1.4 gm, 3.51 mmol) was added to a 50 mL round bottom flask and purged with argon for 3 times (10 mins each). 25 mL of anhydrous dichloromethane was added followed by triethylamine (1.5 equiv., 734 μL, 5.265 mmol) and methacryloyl chloride (1.5 equiv., 509.6 μL, 5.265 mmol) as a dropwise manner under argon atmosphere. The reaction was stirred for 5-6 hours followed by purification on a 40 gm ISCO silica column using dichloromethane:ultra (22% MeOH in DCM with 3% NH₄OH) mixture 0% to 40% to obtain ˜1 gm of colorless solid (Met-Z1A3). The compound was characterized my mass spectroscopy and NMR to confirm the mass and purity.

Synthesis of methacryloyl B2-A17 (Met-B2-A17): (3-(4-(puridin-2-yl)-1H-1,2,3-triazol-1-yl)phenyl)methanamine) (1.2 gm, 4.78 mmol, B2-A17) was added to a 50 mL round bottom flask and purged with argon three times (10 mins each). 25 mL of anhydrous dichloromethane (30 mL) was added, followed by triethylamine (1.5 equiv., 7.17 mmol, 999.6 L) and methacryloyl chloride (1.5 equiv., 7.17 mmol, 693.99 μL) in a dropwise manner under argon atmosphere. The reaction was stirred for 5-6 hours followed by purification on a 40 gm ISCO silica column using hexane/ethyl acetate (1:0 to 0:1) to obtain ˜1 gm of colorless solid (Met-B2-A17). The compound was characterized by mass spectroscopy and NMR to confirm the mass and purity.

Functionalization of Alginates with Small Molecules:

Functionalization of alginate with Z4-A10 amine: 1.5 g of UP-VLVG (1 equiv) was dissolved in 45 mL of water. Then 7.65 mmol of the Z4-A10 (1 eq) amine was dissolved in 22.5 mL acetonitrile and added to the mixture. Following it, an aqueous solution of 4-(4,6-Dimethoxy-1,3,5-triazin-2-yl)-4-methylmorpholinium chloride (0.75 eq) was added dropwise to the mixture. The reaction was stirred overnight at 55° C. The solvent was removed under reduced pressure, and the solid was dissolved in water. The solution was filtered through a pad of cyano-functionalized silica. The solution was then dialyzed against a 10,000 MWCO pretreated dialysis tubing in DI water for three days. The dialyzed solution was frozen at −80° C. and lyophilized until dry.

Functionalization of alginate with B1-A51 amine: 1.5 g of UP-VLVG (1 equiv) was dissolved in 45 mL of water. Then 7.65 mmol of the B1-A51 (1 eq) amine was dissolved in 22.5 mL acetonitrile and added to the mixture. Following it, an aqueous solution of 4-(4,6-Dimethoxy-1,3,5-triazin-2-yl)-4-methylmorpholinium chloride (0.75 eq) was added dropwise to the mixture. The reaction was stirred overnight at 55° C. The solvent was removed under reduced pressure, and the solid was dissolved in water. The solution was filtered through a pad of cyano-functionalized silica. The solution was then dialyzed against a 10,000 MWCO pretreated dialysis tubing in DI water for three days. The dialyzed solution was frozen at −80° C. and lyophilized until dry.

Functionalization of alginate with Z1-A34 amine: 2 g (1 eq) of UP-VLVG (BP-1903-04; Novamatrix) was dissolved in water (75 mL). Then Z1-A34 small molecule (3.99 g, 10.20 mmol, 1 eq) was dissolved in water under vortexing. The pH of the Z1-A34 solution is adjusted to 7.4 using HCl. Then Z1-A34 aqueous solution is slowly added to the UP-VLVG solution while stirring. Then a solution of (4-(4,6-dimethoxy-1,3,5-triazin-2-yl)-4-methyl-morpholinium chloride) (DMTMM, 0.5 eq.) was added dropwise to the mixture of UP-VLVG and Z1-A34. The reaction was heated to 55° C. and stirred overnight. The solution was filtered through a pad of cyano-functionalized silica. The solution was then dialyzed against a 10,000 MWCO pretreated dialysis tubing in a beaker using saline (2 days) and mili-Q water (3 days). The dialyzed solution was frozen at −80° C. and lyophilized until dry.

Gelation assay. The 211 alginate analogs were tested for their ability to efficiently crosslink in the presence of barium to form a hydrogel in a fluorophore retention assay. 100 l of a 1% (w/v) solution of modified alginate polymers in 0.9% saline were dispensed into a 96-well plate. 1 μl of a 1% (w/v) solution of rhodamine B in DMSO was added to each well; followed by 50 μl of a 1 M barium chloride solution then incubated for 10 min on an orbital shaker. The wells were washed 3× with deionized water followed by fluorescence measurements (ex: 540 nm/em: 580 nm). A formerly synthesized UPVLVG alginate will be used as a positive control; deionized water as a negative control.

Coating of Catheters with Met-Z1-A3/Met-B2-A17 using Surface Plasma Cleaning. Barium impregnated silicone rubber catheters (Codman® HOLTER® Atrial Distal catheter, Ref #821670) catheters were cut into identical length pieces using a surgical blade. To perform the chemical modifications, the catheters were plasma treated for three 1 min intervals, rotating between exposures to cover all sides, using high RF frequency at 600-700 mbar pressure (Expanded Plasma Cleaner, Harrick Plasma, P/N PDC-001) and immediately dropped into a 0.02 M solution of Met-Z1-A3/Met-B2-A17 in 5% DMSO in toluene. The reaction was kept under stirring for 2 hours and the implants were thoroughly washed three times in methanol, three times in ethanol, three times with sterile grade water, and finally again with sterile grade ethanol. Finally, the implants were vacuum dried for overnight.

### Scanning Electron Microscopy of Coated and Unmodified Catheters

An unmodified catheter sample was taped to an SEM pin mount, lightly treated with an air gun to remove particulates, and Au sputtered using a Denton Desk V Sputter system (Rice SEA). This was repeated for each coated catheter group. The sputtered catheters were imaged using a JEOL 6500F Scanning Electron Microscope.

### XPS of Unmodified and Coated Catheters

X-ray photoelectron spectroscopy (XPS) is a surface-sensitive spectroscopic method that quantitatively measures the elemental composition at the surface (within 6 nm range) of any material when the sample is irradiated mono-energetic X-rays causing emissions of photoelectrons from the material's surface. The elemental compositions of uncoated, Met-Z1-A3 coated, Met-B2-A17 coated implants were analyzed by PHI Quantera XPS. A Survey technique at 1100 eV, 200 m spot size, 50 W 15 kV ion gun neutralization was used for the analysis.

### ToF-SIMS of Unmodified and Coated Catheters

Positive and negative high mass resolution spectra were performed using a ToF-SIMS NCS instrument, which combines a TOF.SIMS5 instrument (ION-TOF GmbH, Munster, Germany) and an in-situ Scanning Probe Microscope (NanoScan, Switzerland) at Shared Equipment Authority from Rice University. A bunched 30 keV Bi₃⁺ ions (with a measured current of 0.2 pA) was used as a primary probe for analyzing a field of view of 250×250 μm², with a raster of 128 by 2128 pixels and by respecting the static limit of 1.10¹² ions/cm² not to damage the surface. A charge compensation with an electron flood gun has been applied during the analysis, and an adjustment of the charge effects has been operated using a surface potential. The cycle time was fixed to 100 s (corresponding to m/z=0-1102 a.m.u mass range).

Material Characterization and Evaluation of Catheters Coated with Lead Molecules.

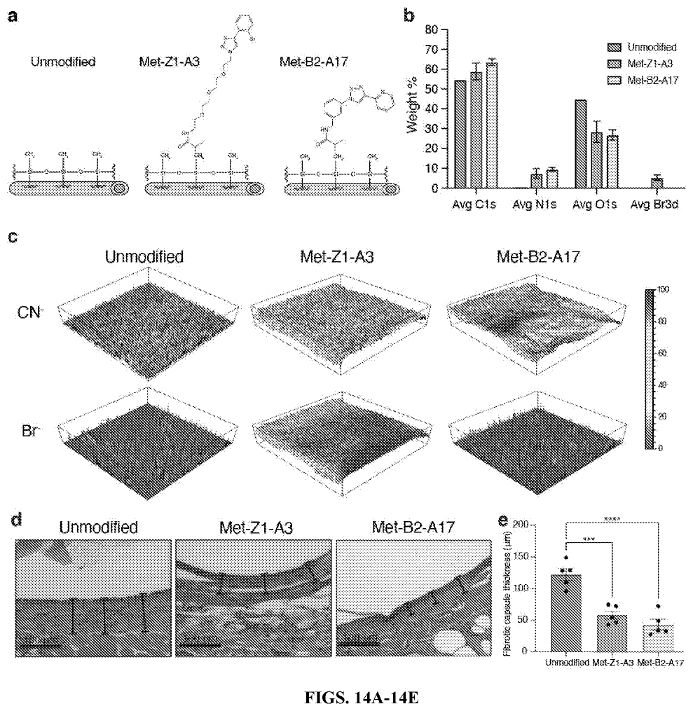

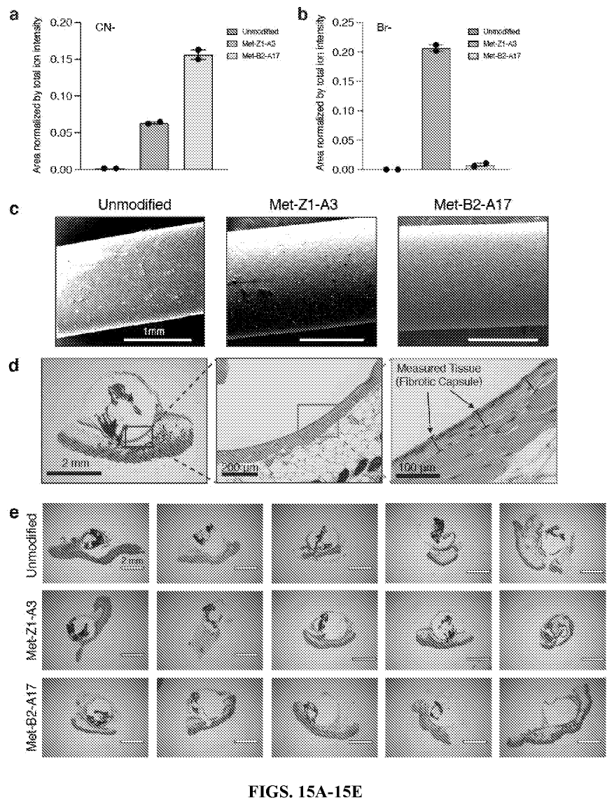

XPS results showed higher nitrogen content in catheters coated with the small molecules than uncoated catheters, suggesting an effective attachment of nitrogen-containing small molecules to the catheters (FIG. 14B). Moreover, the Br⁻ content increase in bromine-containing Z1-A3 coated catheters supports the attachment of small molecules to the catheter surface. The XPS results were further supported by Tof-SIMS results showing higher CN⁻ content in catheters coated with small molecules and higher Br⁻ content in bromine-containing Z1-A3 coated catheters (FIG. 14C, FIG. 15A, FIG. 15B) compared to uncoated ones. Additionally, the Met-Z1-A3 and Met-B2-A17 catheters appeared smoother than the unmodified catheter observed from the SEM images (FIG. 15C), possibly due to the removal of microscopic bumps during the coating process.

Cell culture and expansion. Human umbilical vein endothelial cells (HUVECs, CC-2517, LONZA, MD, USA) from 20 different donors (Table 3) were cultured in VascuLife® VEGF medium complete kit (LL-0003, Lifeline Cell Technology, CA, USA). The HUVECs were sub-cultured for expansion and maintained in a humidified incubator at 37° C. in a 5% CO₂ atmosphere. The media was changed 3 times every week.

### 20 Donor's SNP Profiles

A 30-plex single nucleotide polymorphism (SNP) panel was designed to uniquely identify HUVEC donors through multiplex PCR reactions and next-generation sequencing (NGS). The SNP loci were downloaded from the 1000 Genomes database, and the genomic sequences were fetched from the National Center for Biotechnology Information (NCBI) website. The SNP were selected such that the minor allele's population allele frequency 10% and 90%, and their loci evenly distributed across chromosomes 1-22.

Genomic DNA (gDNA) was extracted from 20 different donors of HUVECs using a DNeasy kit (Qiagen, catalog #69054). For each donor, 100 ng of gDNA was added to 50 μL of PCR reaction mix with a concentration of 50 nM for each primer and Phusion Hot Start Flex 2× Master Mix (NEB, catalog #M0536L). The PCR reaction condition included an activation at 98° C. for 30 sec, and 20 cycles of denaturation at 98° C. for 30 sec, annealing at 63° C. for 2 min, and extension at 72° C. for 1 min and completed the reaction with incubation at 72° C. for 5 min (shorten as 98° C.: 30 s-(98° C.:10 s-63° C.:2 min-72° C.:1 min)×20-72° C.:5 min-4° C.: hold). The PCR products were purified using Monarch PCR & DNA Cleanup Kit (5 μg) (NEB, catalog #T1030S). The purified PCR products were then prepared for NGS on Illumina Miseq platform using NEBNext Ultra II DNA Library Prep Kit for Illumina (NEB, catalog #E7645S) according to the manufacturer's protocol. The library quantification and quality control were performed on Agilent 2100 Bioanalyzer. Then deep sequencing was performed at approximately 5000× depth to establish SNP profile for 20 different HUVEC donors.

HUVEC Encapsulation within Modified Alginate.

Capsule fabrication for Mice. For each round screening, twenty different materials were tested, and a total of 149 materials were screened in C57BL/6J mice. Therefore, every round, twenty different HUVECs donors were used to create corresponded cell-barcodes for each material. Modified alginates initially dissolved at 3-5% w/v in 0.8% saline and then blended with 3% w/v SLG100 (also dissolved in 0.8% saline) at a volume ratio of 70% modified alginate to 30% SLG100. Alginate solutions were sterilized by filtration through a 0.2-μm filter. Immediately prior to encapsulation, the cultured HUVECs were centrifuged at 250 G for 5 minutes and washed with Ca-free Krebs buffer (4.7 mM KCl, 25 mM HEPES, 1.2 mM KH2PO4, 1.2 mM MgSO4·7H2O, 135 mM NaCl). After washing, cells were centrifuged again, and all supernatants was aspirated. The cell pellet was then re-suspended in alginate solution at the cell density of 5×10⁶ cells per 0.5 ml alginate solution (˜40,000 cells/capsule). Each HUVECs donors were encapsulated with corresponding modified alginate solutions. Alginate capsules were made using an electro-spraying machine (Pump 11 Pico Plus, Harvard Apparatus, MA, USA). 18 G blunt-tipped needle is attached to a 1-ml Luer-lock syringe containing the alginate solution, which is clipped to a syringe pump that is oriented vertically over a 150 ml of crosslinking solution bath (20 mM BaCl2, 250 mM D-Mannitol, 25 mM HEPES with 0.01 v/v % tween 20). A voltage generator was attached to the needle tip and grounded to the crosslinking bath. The settings of the syringe pump were 5 ml/hr flow rate at 15-20 cm height. By adjusting a voltage between 5.5 and 7 kV, cell density per capsule was maintained consistently. After the capsules are formed in the crosslinking bath, they are then collected and then washed 3 times with HEPES buffer (25 mM HEPES (Gibco, Life Technologies, California, USA), 1.2 mM MgCl2×6H2O, 4.7 mM KCl, 132 mM NaCl) and washed 3 times with medium, and cultured overnight at 37° C. incubator for transplantation. 10 capsules of each material were aliquoted and mixed into one 2 ml tube, and this mixture of 200 capsules were used for implantation. Immediately prior to implantation into the peritoneal cavity of mice, the capsules were washed an additional two times with 0.9% saline. All materials were observed under bright-field microscopy to verify homogenous cell density and size of capsules.

For microcapsules implantation, top ten lead materials identified from mice screening and two controls (SLG20 and Z1-A34) were used. Modified alginates were dissolved at 3˜5% w/v in 0.8% saline and blended with 3% w/v SLG 100 at 70:30 ratio. SLG20 were dissolved at 1.4% w/v in 0.8 saline. Formulated alginate solutions were used to make 300˜500 μm size capsules. Encapsulation procedures were same with 1.5 mm size capsules except that 30 G needle was used for microcapsules with 200 μL/min flow rate. After washing, 400 μL of microcapsules were aliquoted and implanted into mice IP cavity space for 2 weeks.

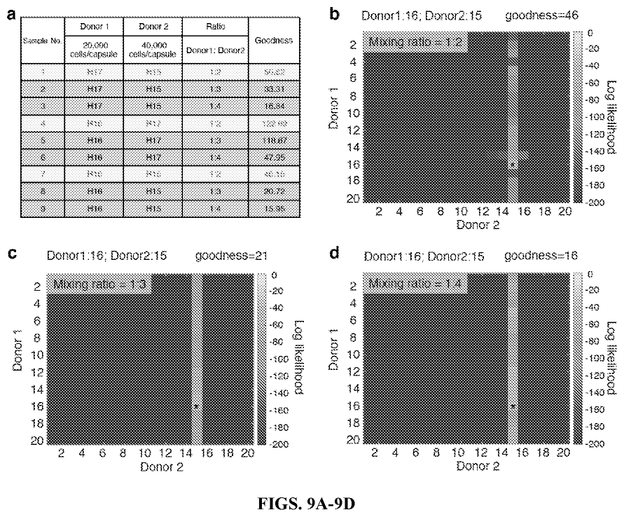

Capsule fabrication for NHP implant: By mixing two different HUVECs donors at specific ratios, 400 different donor combinations from twenty different donors can be created to tag the biomaterials. To confirm the feasibility of mixed donor identification, different ratios (1:2, 1:3, and 1:4) were tested and determined what ratios are detectable via NGS. A total of nine combinations were made with the three cell lines (FIG. 9A). Once the correct ratios were aliquoted in their respective test tubes, nine different ratio capsules were created using SLG20, followed by the encapsulation protocol mentioned in the previous section. Including 20 single HUVECs donors, a total of 400 combinations of cells barcodes can be generated, and 100 cell barcodes among 400 were used for the NHP study. A total of 100 cell barcodes were created, and these mixtures of HUVECs were encapsulated with 100 different materials, respectively. Cell density per capsule was fixed at 60,000 cells/capsule, which included 20,000 cells of donor 1 and 40,000 cells of donor 2, following the same encapsulation method described in the previous section (mouse study). Thirty capsules per material were mixed into one T225 flask, and these mixed capsules were prepared for IP implantation. After the aliquoting desired number of capsules into flasks, these samples were shipped overnight for implantation in an NHP.

### Optimization of Donor Barcoding and Identification

By combining two different donors from 20 donors, a total of 400 donor pairs was created as described above. First, to test the feasibility of mixed donors, the blend ratio of two donors was optimized in vitro. Three donor combinations (H15&H16, H16&H17, H15&H17) with three different blend ratios (1:2, 1:3, 1:4) were used for this study, and subsequently, a total of 9 different conditions were tested (FIG. 9A). One capsule from each group was mixed, and the donor pairs of 9 capsules were analyzed (sample no. 1-9). Three different mixing ratios were tested to find the optimal condition with higher identification confidence (goodness levels). Log-likelihood analysis was applied to deconvolute donor identity. All samples were successfully identified with their matching donor pairs. The goodness level decreased as the cell density of the two mixing cells was more apart from one another. The blend ratio of donor1:donor2 was set to 1:2 as optimal conditions for further experiments (FIG. 9B-9D).

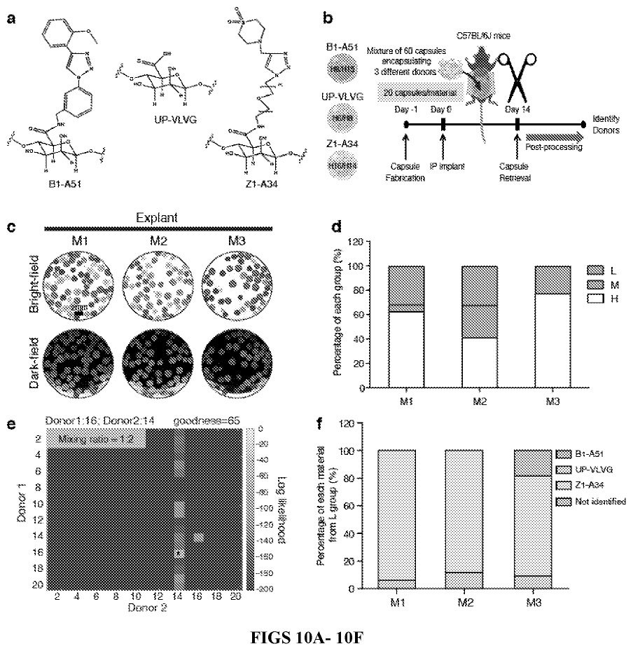

Next, with the optimized condition in vitro, the identification feasibility with three different materials was tested in mice. Three materials (Z1-A34: positive control mitigating FBRs, PVLVG: unmodified control, and B1-A51: a negative control unable to mitigate fibrosis) encapsulating two donor pairs at 1:2 ratios were prepared. The mixtures of three materials (20 capsules/material for each mouse) were implanted into IP space in mice (M1-M3) for two weeks (FIG. 10A & FIG. 10B). After retrieving capsules (FIG. 10C), they were separated into three groups based on fibrosis levels (FIG. 10D). A total of 45 capsules were selected for donor identification, and the corresponding materials were determined (FIG. 10E & FIG. 10F). 43/45 (95.6%) of donor pairs were successfully identified among 400 donor combinations, suggesting that this mixed donor barcoding strategy can be applied to screen hundreds of materials in a larger animal model. The result of this cohort showed that previously published Z1-A34 (immune protective material) has the highest numbers of low fibrosis capsules, following UP-VLVG (unmodified alginate). The ability to screen hundreds of materials with this dual barcoding strategy will exponentially extend the high-throughput potential of the screening to identify materials mitigating FBRs.

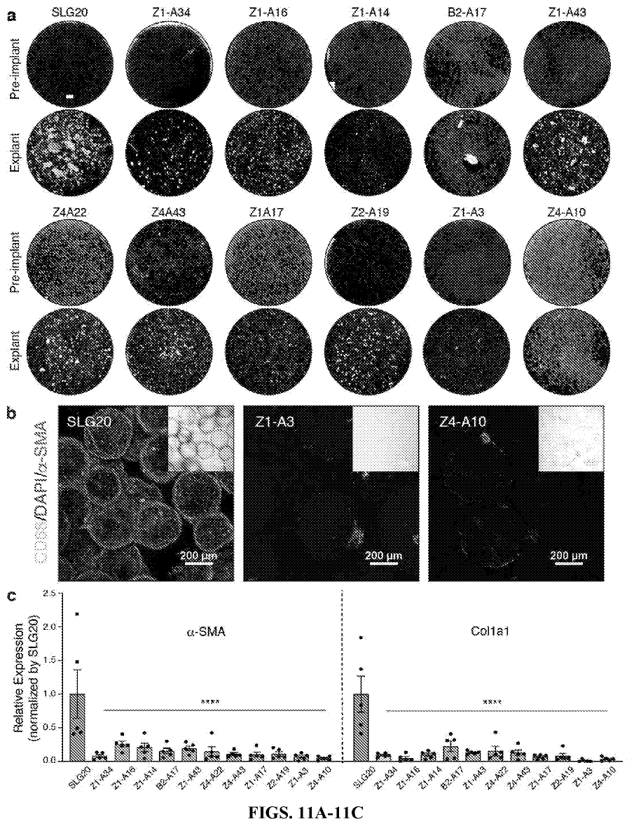

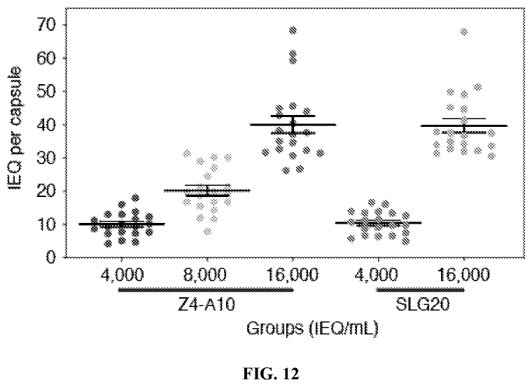

Human islet encapsulation with lead material: Human islets (from Prodo Labs) were cultured in PIM(S) media (Prodo Labs) for further use. The cultured islets were centrifuged at 1200 rpm for 3 mins and washed with Ca-free Krebs buffer. The islets were then centrifuged again. The islet pellet was then resuspended in a 5% solution of Z4-A10 (blended with 3% SLG100 at 70:30 ratio) at an islet density of 5,000 islets per 1 mL alginate solution. Capsules were crosslinked in BaCl₂ solution, and their sizes were adjusted to 1.5 mm. After crosslinking, the capsules were washed with HEPES three times and washed again with media twice. As the islets had variable sizes (50˜400 μm), the total number of encapsulated islets were recounted and converted into islets equivalents (IEQ) (FIG. 12). The average IEQ of each capsule was 10 IEQ/capsule (1×). To make high-density capsules (2× and 4× density), alginate volume was reduced to 0.5 mL and 0.25 mL, respectively, while keeping the same IEQ. Finally, ˜20 IEQ/capsule (2×) and ˜40 IEQ/capsule (4×) were fabricated with Z4-A10 modified alginate. SLG20 was used as a control material, followed by the same encapsulation method and islets density.

Optimization of DNA extraction (in vitro): To increase the amount of extracted gDNA from a single capsule, different parameters were optimized using a DNesay kit (Qiagen, catalog #69054). Fresh capsules and flash-frozen capsules were compared with or without lysis conditions. 50 mM EDTA in 10 mM HEPES solution was applied to lyse capsules and then centrifuged at 250 G for 5 mins. Only pelleted cells were used for the following DNA extraction process. Un-lysed capsules were also used after homogenization as a comparison. Second, the yield was tested against different elution temperature settings (RT vs. 56° C.). Finally, different cell numbers per capsule (5,000, 10,000, 20,000, 40,000, and 80,000 cells per capsule) were compared to determine optimal cell density for in vivo study.

Optimization of DNA Extraction from a Single Capsule and NGS Identification

Since individual hydrogel capsules contain unique genotypes from different HUVECs, extracting gDNA from each capsule is an essential first step to identifying each material's barcode.

Cells are present within crosslinked hydrogel matrix, which makes it challenging to isolate cells from the hydrogel. Cell isolations and gDNA extraction steps were optimized to increase gDNA content by comparing lysis compositions, cell density, and extraction temperature (FIG. 4). Pre-implant HUVECs capsules were used to compare DNA extraction efficiency in different conditions. First, each capsule was dissociated with EDTA solution. Lysis of capsules with EDTA before extracting DNA from a single capsule improved DNA extraction efficiency ˜5-fold (FIG. 4A). Also, flash-frozen capsules showed a similar DNA content level with fresh samples, confirming that explanted capsules can be stored for future use after flash freezing steps. Also, different elution conditions were compared to increase the extracted DNA amount (FIG. 4B). When samples were eluted with pre-warmed elution buffer at 56° C., DNA content was increased. In addition, as elution volume increased, the total DNA amount increased. However, increasing elution volume reduced DNA concentration, and 100 μl of elution buffer was chosen as the optimal volume for further analysis. Finally, cell numbers per capsule were adjusted by comparing different cell density conditions (FIG. 4C). Extracted DNA content was elevated as cell density per capsule was increased. However, total DNA yield was decreased, so ˜20,000 cells/capsule was set as the minimum cell density for encapsulation. The optimized conditions were applied to extract gDNA from explanted capsules, and these extracted gDNA samples were used for optimizing NGS methods.

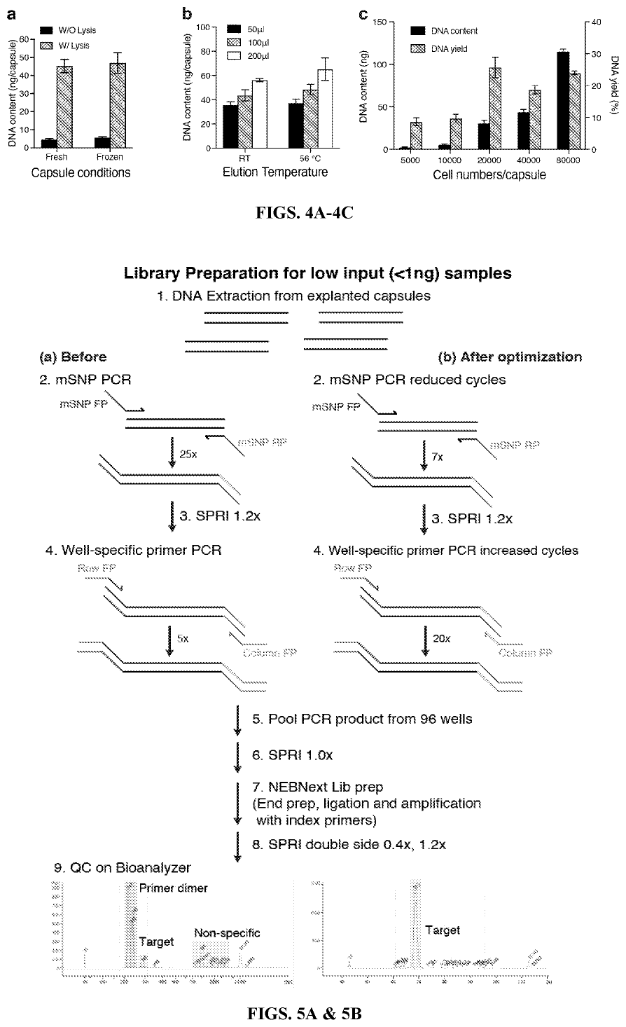

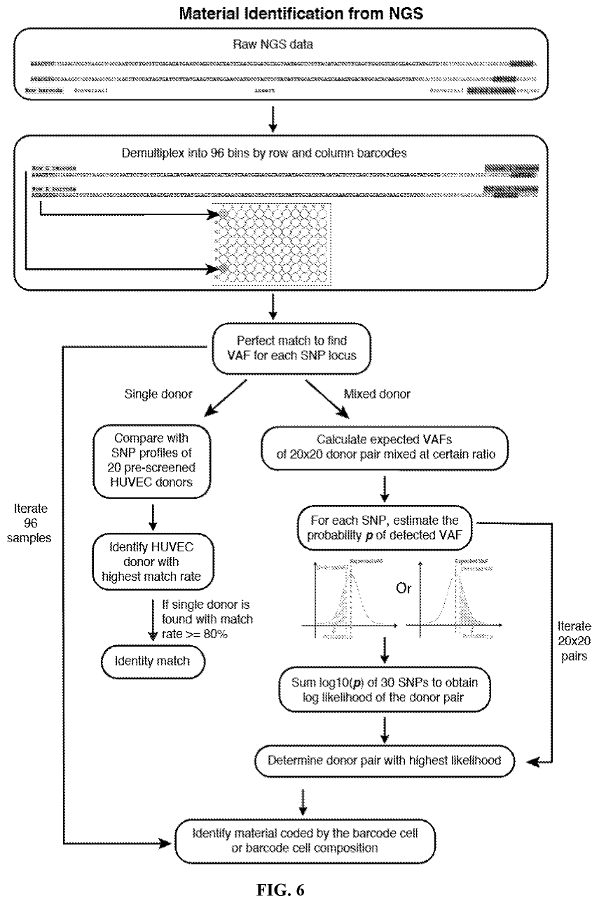

Successful DNA extraction was done from in vitro and in vivo explanted capsules with high quality and enough human DNA content for polymerase chain reaction (PCR) amplification, followed by NGS for SNP genotyping. Furthermore, NGS library preparation was optimized to increase the on-target rate even with low DNA input (FIG. 5). Finally, the bioinformatic pipeline shows the high-throughput strategy of the NGS analysis process for material/donor identification (FIG. 6).

Optimization of NGS library preparation workflow: Because DNA extracted from capsules was at low concentration, and the input for constructing the NGS library is typically below 1 ng, the PCR reactions were more prone to primer-dimer, especially in multiplex PCR. Here, the library preparation workflow was optimized to reduce primer-dimer. The first PCR amplified SNPs with multiplex primers containing 5′-overhang sequences. The primer concentration, PCR cycles, and annealing time of the first PCR were adjusted to reduce primer-dimers that could arise from multiplex PCR. Following on-plate purification, the second PCR amended position barcodes by amplifying with row-specific and column-specific primers comprising hamming barcode sequence and sequence that annealed to the 5′-overhang region of SNP primers. The amplification cycles of the second PCR were also adjusted.

### Implantation/Transplantation Surgeries.

IP implant of mixed capsules in C57BL/6J mice: All mice experiments were approved by Rice University's Institution Animal Care and Use Committee (IACUC). Immune-competent male C57BL/6J mice were first weighed and anesthetized with 14% isoflurane in oxygen at a heating pad. Buprenorphine was administered subcutaneously based off their weight (0.5 mg/kg dose). Their abdomens were shaved and sterilized using betadine and isopropanol scrubbing 3 times, respectively. A 0.5-10 cm midline incision through the skin was made using a sharp blade. The peritoneal wall was then grasped with forceps and a 5 mm incision was made along the linea alba. A volume of 0.5 ml of capsules was then implanted into the peritoneal cavity. The abdominal muscle was closed using absorbable sutures. The skin was then closed with suture.

Subcutaneous Implant of Catheter Samples in C57BL/6 Mice: Unmodified and coated catheters were implanted in the subcutaneous space of n=6 C57BL/6 mice (Charles River Labs). Specifically, one incision site was made in the back of each mouse, and one separate subcutaneous pocket was made for each catheter implant. The incisions were sutured closed, and the mice were monitored and cared for according to Rice Animal Resource Facility standards.

IP implant of human islets capsules in STZ-induced diabetic C57BL/6J mice and blood glucose monitoring: To create insulin-dependent diabetic mice, healthy C57BL/6J mice were treated with streptozotocin (STZ). For five consecutive days, STZ solution at 7.5 mg/ml concentration (50 mg/kg of STZ) was injected in IP space. The blood glucose (BG) levels and weights of all the mice were measured after one hour of fasting. Only mice whose BG levels were above 350 mg/dL for two consecutive days were considered diabetic and used for islets transplantation. Two hundred capsules containing human islets were implanted into diabetic mice (˜2,000 IEQ per mouse) for 1× group. Furthermore, 100 capsules at ˜20 IEQ/capsule and 50 capsules at ˜40 IEQ/capsule were implanted for 2× and 4× groups, keeping the same IEQ number per mouse (˜2,000 IEQ per mouse). BG levels were monitored three times a week following transplantation of islet containing Z4-A10 and SLG20 capsules. Mice with BG levels below 250 mg/dL were considered normoglycemic. Monitoring continued until all mice had returned to a hyperglycemic state, at which point they were euthanized, and the capsules were retrieved. Mice fasted for 4 hrs before in vivo glucose tolerance test. Each mouse was given a bolus dose of 30% sterile glucose solution in saline at 1.5 g/kg through a tail vein injection. Blood glucose levels were measured every 15 mins for 2 hrs after the glucose injection.

Implantation of capsules laparoscopically into IP space in NHP: On the day of the scheduled surgery, the NHP (male Mauritian cynomolgus monkey) was sedated and anesthetized (as per approved animal protocol). The anterior abdomen was shaved and prepped from xyphoid to pubis. A small (2 cm) supraumbilitical incision was performed, and a 5-12 mm trocar was inserted. Pneumoperitoneum was created with CO₂ at a pressure of 10-14 mmHg. After warming up with heated saline, the camera was inserted into the peritoneal cavity through the trocar. Under the view of laparoscopy, another 2 small incisions (1-2 cm) were made (on the left and right flank), and 5-12 mm trocars were inserted into the peritoneal cavity. A 2 mL sterile pipette connected to the syringe by a silicone tube was inserted through the trocar to the abdominal cavity. The capsules were distributed evenly in the following spaces: perihepatic, retrogastric, perisplenic, left and colonflexium, omentum, and behind small bowels. The three small incisions were then sutured by layered closure using Vicryl 3-0 for muscle and 4-0 Vicryl for skin (subcuticular). The animal's recovery was followed by the OR staff, veterinary staff, and technicians. The animal protocol involving the care and use of non-human primates in this study was reviewed and approved by the University of Illinois-Chicago (UIC) Institutional Animal Care and Use Committee (IACUC) prior to commencement. All animal procedures were performed in accordance with the Guidelines for Care and Use of Laboratory Animals of UIC.

### Retrieval of Materials.

Capsule retrieval from IP space: At a specific period of implantation, five mice from each round were euthanized under CO₂ administration, followed by cervical dislocation. An incision was then made using the forceps and scissors along the abdomen skin and peritoneal wall. Ca+ Krebs buffer was then used to wash out all material capsules from the abdomen and into Petri dishes for collection. After ensuring all the capsules were washed out or manually retrieved, they were transferred into 50 ml conical tubes. After several washing steps of Krebs buffer, the mixed explanted capsules were processed for further imaging and selection.

Capsule retrieval from the IP cavity of NHPs via laparoscopic technique: NHPs were sedated and anesthetized (as per approved protocol). The anterior abdomen was shaved and prepped from xiphoid to pubis. A 2 cm supraumbilical incision was performed, and a 5-12 mm trocar was inserted. Pneumoperitoneum was created with CO₂ at a pressure of 10-14 mmHg. After warming up with heated saline, the camera was inserted into the peritoneal cavity through the trocar. Under the view of laparoscopy, another incision (1-2 cm) was made (on the left or right flank), and a 5-12 mm trocar was inserted into the peritoneal cavity. Laparoscopic images and video was taken to record microcapsule distribution. 20-30 cc of saline was used for flushing any free capsules laparoscopically that may be present in the pelvic space that may have leaked from the lesser sac. The animal protocol involving the care and use of non-human primates in this study was reviewed and approved by the University of Illinois-Chicago (UIC) Institutional Animal Care and Use Committee (IACUC) prior to commencement. All animal procedures were performed in accordance with the Guidelines for Care and Use of Laboratory Animals of UIC.

Processing, Fixation, and Histology of Catheter Explants: Catheters were explanted at 4 weeks by carefully removing the catheters and attached tissues together. The subcutaneous catheter explants were strongly attached to the skin and were covered with a thin membrane that lightly adhered to the muscle (FIG. 15). The tissue around the catheter was cut about 3 mm from the catheter, and the catheter with skin attached was removed from the mouse. Explants were fixed with 10% formalin (Sigma) for four days before being transferred to PBS. Further processing, sectioning, and histology was done by the Baylor Pathology and Histology Core. Specifically, samples were paraffin embedded, sectioned along the cross-sectional axis of the catheters, and stained with H&E stain.

Imaging and selection of the explanted material capsules: For phase-contrast imaging, capsules were gently washed using Krebs buffer and transferred into 35 mm Petri dishes for bright- and dark-field imaging using Leica microscope. Based on the levels of fibrotic overgrowth onto the capsule surface, capsules were manually separated into three different groups ((L: Low, M: Medium, and H: High fibrosed groups). Selected clear capsules with low fibrosis (L group) were flash-frozen and used for DNA extraction, otherwise left capsules were flash-frozen with liquid N₂ and stored at −80° C. for further uses.

Live/dead cells staining: Fluorescent imaging of cells stained with live/dead assay was performed to check encapsulated HUVECs viability from either pre- or post-implant capsules. 5 capsules of each material were washed with DPBS and stained with 2 μM calcein AM and 4 μM EthD-1 in complete media. Capsules were incubated for 30 minutes and imaged using an EVOS microscope with fluorescence filters. Live cells were imaged with a GFP filter as green and dead cells were imaged with a Texas-Red filter as red color. (Explant capsules-NSG mice) In case of explanted capsules from NSG mice, capsules were washed three times with Ca+ Krebs buffer and then incubated in staining solution. Capsules from each mouse were transferred into 35 mm petri dish and washed twice with DPBS. They were imaged under 2× magnification and acquired images were stitched to observe entire dish.

Dithizone staining: The explanted islets capsules were stained with dithizone (DTZ). 5 mg of DTZ was dissolved in 1 mL of dimethyl sulfoxide (DMSO), thoroughly mixed, and incubated for 5 mins. 4 mL of DPBS was added into the mixed solution and filtered through a 0.22 m filter. Islets capsules were placed in a 35 mm Petri dish and washed with PBS three times. Capsules were then incubated in DTZ solution for 5 mins and washed with DPBS three times to remove background staining. The stained capsules were imaged with a Leica microscope.

Protein extraction and collagen content quantification from retrieved microcapsules: Cells/tissues deposited on microcapsules' surface were lysed using RIPA buffer (Cat #89901, Thermo Scientific, PA, USA) for protein extraction. Briefly, a ratio of 100 μl microcapsules to 200 μl lysis buffer with Halt™ Protease Inhibitor Cocktail (Cat #78430, Thermo Scientific, PA, USA) was used for cell lysis from capsules. Lysate were centrifuged for 20 min at 12000 rpm at 4° C. and the supernatant was transferred into a new tube. The pellets were washed with the same volume of lysis buffer, and then centrifuged for 20 min at 12000 rpm at 4° C. The supernatant was combined with the previous one and the extracted protein were stored at −80° C. for future use. Protein concentration in the lysate was quantified using BCA assay (Pierce BCA Protein Assay Kit, Cat #23225, Thermo Scientific, PA, USA). Lysate from each sample containing a quantity of 20 μg of protein was diluted with water up to 100 μL, mixed with 37% hydrochloric acid at 1:1 ratio, and then hydrolyzed at 120° C. for 3 hrs. The resulting solution was used to determine the collagen content of retrieved microcapsules using a hydroxyproline assay kit (Cat #MAK008, Sigma-Aldrich, MO, USA) according to the manufacturer's instructions. The absorbance at 560 nm was measured, and the value at blank of hydroxyproline standard was subtracted from all readings. The hydroxyproline content was determined from the hydroxyproline standard curve.

Immunofluorescence staining for confocal imaging: For immunofluorescence staining, retrieved microcapsules were washed with Krebs buffer and fixed in 4% paraformaldehyde overnight at 4° C. Samples were washed with PBS three times, and cells were permeabilized with a 1% Triton X-100 for 15 mins at room temperature. After washing with PBS, samples were incubated in 1% bovine serum albumin (BSA) solution for 1 hr at room temperature for blocking, and then incubated with staining solution containing the antibody cocktails (diluted at 1:200 Alexa Fluor 488 anti-mouse CD68 Antibody (Cat #137012, BioLegend, CA, USA), 1:200 Anti-mouse α-Smooth Muscle-Cy3 (Cat #C6198, Sigma-Aldrich, MO, USA) in 1% BSA, and 2 drops/ml of DAPI (NucBlue Fixed Cell ReadyProbes Reagent, Cat #R37606, Invitrogen, CA, USA)) for 1 hr at room temperature. After washing with 0.1% tween 20 solution, samples were washed twice with PBS, and transferred to a 50% glycerol solution in a glass-bottomed 24-well plate for imaging. Nikon A1-Rsi confocal microscope was used for immunofluorescence imaging.

Histological processing (H&E and Masson's Trichrome staining) of retrieved catheters and histology-based assessment of tissue overgrowth on catheters: Stained sections of catheter explants were imaged with a Leica M165C light microscope. To determine the extent of tissue overgrowth on the catheters, the dark band of purple tissue between the catheter and skin layer was measured using ImageJ software. This method is based on the findings of Xie et. al., which show the tissue band directly adjacent to the subcutaneous implant stains dark purple with H&E and stains blue with Trichrome (Xie et al., 2018). Staining blue with Trichrom means that this tissue band is collagen rich, which in turn suggest this tissue is indicative of fibrotic overgrowth. Because of this correlation, the dark purple tissue was measured and used to assess the relative immune response to the differently coated catheters.

Specifically, the catheter section image was rotated to angle the adjacent skin tissue downwards. Three measurements, which were equally spaced along the skin tissue, were taken of the dark purple band of tissue for each image and averaged to avoid arbitrarily measuring one spot. Measuring the length of the scale bar burned into the image was used to convert the ImageJ pixel measurement to millimeters. Note that some sections have large, lighter purple bands of tissue that run more perpendicular to the catheter. These were specifically avoided during measurements since they are part of scar tissue from the incision.

DNA extraction from explanted capsules and RT-qPCR. For DNA extraction from a single capsule, the optimized method was applied for this study. Encapsulated cells from pre- or post-explant capsules were lysed in 50 mM EDTA for 15 mins and centrifuged at 5000 rpm for 10 mins. The supernatant was aspirated, and the cell pellet was suspended in 200 μl of PBS. Total gDNA from a single capsule was isolated using the DNeasy kit (Qiagen, catalog #69504) according to the manufacturer's instructions with small modifications as optimized conditions. Briefly, cell suspension was lysed with proteinase K and RNase A for 5 mins at RT, and incubated with lysis buffer for 20 mins at 56° C. After the addition of ethanol, the supernatant was transferred into a column and washed twice with a wash buffer. DNA was collected with elution buffer heated at 56° C. and store at −20° C. for further uses. Total RNA was extracted from 100 μl of retrieved microcapsules (300-400 μm size). Snap frozen capsules were thaw on ice, homogenized, and processed using RNeasy Mini Kit (Qiagen, catalog #74104) according to the manufacturer's instructions. Extracted RNA was converted to cDNA for RT-qPCR using the high-capacity cDNA reverse transcription kit (Applied Biosystems, catalog #4368814). Real-time qPCR was performed using 3 μL of gDNA/cDNA in a 10 μL reaction volume with SYBR Green (PowerUp SYBR Green Master Mix; Applied Biosystems, catalog #A25741) to quantify PCR product. PCRs were carried out under the following conditions: 95° C. for 10 s, 48° C. for 20 s, 72° C. for 30 s (40 cycles), 72° C. for 5 min, 65° C. for 5 s, and a final cycle at 95° C. All reactions were run in triplicates. Data were analyzed with the 2-ΔΔCT method, and relative RNA levels were compared after normalization to mouse b-actin (ActB) and SLG20 (control). The primers used in this study are listed in Table 6.

TABLE 6
List of RT - qPCR primers

| | | SEQ |
|---|---|---|
| | | ID |
| Target | Primers (5′→3′) | NO: |
| Mouse Alpha | Forward: CGCTTCCGCTGCCCAGAGACT | 1 |
| Smooth muscle | Reverse: | 2 |
| actin (a-SMA) | TATAGGTGGTTTCGTGGATGCCCGCT | |
| Mouse Collagen | Forward: CATGTTCAGCTTTGTGGACCT | 3 |
| 1a1 (Col1a1) | Reverse: GCAGCTGACTTCAGGGATGT | 4 |
| Mouse b-actin | Forward: GCTTCTTTGCAGCTCCTTCGTT | 5 |
| (ActB) | Reverse: CGGAGCCGTTGTCGACGACC | 6 |

NGS Analysis to Identify the Immunomodulatory Alginate Analogue with Low Fibrosis.

NGS library preparation. DNA content in individual capsules was semi-quantitatively evaluated using qPCR as a sample quality control procedure, with Ct values in negative correlation to extracted DNA content. Samples with amplifiable DNA content would go through two PCR steps to amplify and barcode the target amplicons. With DNA from each capsule in individual wells of a 96-well PCR plate, the first PCR was performed using 30-plex primers targeting 30 non-pathogenic SNP whose genotype profile will uniquely identify a HUVEC donor (Table 7). The 30-plex primers all contained a 5′-overhang sequence to incorporate a universal binding domain to target amplicons. The reaction mixture was comprised of 30-plex SNP primers at concentration of 50 nM each and Phusion Hot Start Flex 2× Master Mix at 1×. The reaction condition was activation at 98° C. for 30 s, and 7 cycles of denaturation at 98° C. for 30 s, annealing at 63° C. for 5 min, and extension at 72° C. for 1 min and complete the reaction with incubation at 72° C. for 5 min (shorten as 98° C.:30 s-(98° C.:10 s-63° C.:5 min-72° C.:1 min)×7-72° C.:5 min-4° C.:hold). AMPure XP magnetic beads (Beckman Coulter, catalog #A63881) with a 1.2× volumetric ratio will be added to the first PCR product. The suspension was incubated at room temperature for 5 min at room temperature, then placed on a magnetic stand to separate and discard the supernatant. The remaining magnetic beads were washed twice with 80% ethanol, and DNA content was eluted in water. The second PCR used primers carrying overhang Hamming code sequence to uniquely barcode capsule samples. A total of 12 barcoded column-specific forward primers and 8 barcoded row-specific reverse primers were designed to have a distance of at least 3 from one another that allowed barcoding of 12×8=96 capsules. The reaction condition was 98° C.:30s-(98° C.:10 s-63° C.:1 min-72° C.:1 min)×20-72′C:5 min-4° C.:hold) with primer concentration of 400 nM and Phusion Hot Start Flex 2× Master Mix at 1×. The row and column barcodes uniquely determined sample position on the 96-well plate, and thus barcoded amplicons could be pooled from all wells, forming a single library containing SNP information from 96 different capsule samples (Table 8). The products were purified using AMVPure XIP magnetic beads at 1.0× volumetric ratio, as previously described above.

Illumina sequencing adapters are amended by ligation-based method and library index was added by PCR using NEBNext Ultra II DNA Library Prep Kit for Illumina (NEB, catalog #E7645 S) according to the manufacturer's protocol. The library quantification and quality control was be performed on Agilent 2100 Bioanalyzer.

TABLE 7
SNP information and SNP primer sequences.

| | | Position | | | |
|---|---|---|---|---|---|
| | SNP ID | (GrCh38) | | Primer sequence (5′→3′) | SEQ ID NO: |
| 1 | rs10230708 | 7: 26625472 | Forward | CCAAAGTCGTTAAGCTGCCAAACCAATG | 7 |
| | | | | GGAGTCACTGCTG | |
| | | | Reverse | TATGTCCGTCGTTGCGAAGTGCCTGGACA | 8 |
| | | | | TCAGTGTCCTCATCT | |
| 2 | rs2043583 | 3: 24598078 | Forward | CCAAAGTCGTTAAGCTGCCAACCTGAAT | 9 |
| | | | | GTCAGTTTTGTTAGAGCAAC | |
| | | | Reverse | TATGTCCGTCGTTGCGAAGTGACAGGGT | 10 |
| | | | | GATGTAAAAGTGTCTGA | |
| 3 | rs955456 | 4: 23651593 | Forward | CCAAAGTCGTTAAGCTGCCAACAGACTT | 11 |
| | | | | AATCAAAGCCCTTGAAAAGA | |
| | | | Reverse | TATGTCCGTCGTTGCGAAGTGACTCATGG | 12 |
| | | | | CAACTTGCTTCTCA | |
| 4 | rs966516 | 4: 24775000 | Forward | CCAAAGTCGTTAAGCTGCCAACCTCCCAT | 13 |
| | | | | AGTGATTCTTATGAAGTCA | |
| | | | Reverse | TATGTCCGTCGTTGCGAAGTGGGATAAC | 14 |
| | | | | CTTGTGTGCATGTCACT | |
| 5 | rs11247921 | 1: 26339111 | Forward | CCAAAGTCGTTAAGCTGCCAACCACACT | 15 |
| | | | | CTGCCTCTCATGGTAT | |
| | | | Reverse | TATGTCCGTCGTTGCGAAGTGAGGCAAG | 16 |
| | | | | TAAGCAGACAATAGCA | |
| 6 | rs10510620 | 3: 29288795 | Forward | CCAAAGTCGTTAAGCTGCCAATCCGCAA | 17 |
| | | | | AACCTACAATCTCTGAA | |
| | | | Reverse | TATGTCCGTCGTTGCGAAGTGCAGTATAG | 18 |
| | | | | CTGAGGTAACCACAGTA | |
| 7 | rs3789806 | 7: 140749271 | Forward | CCAAAGTCGTTAAGCTGCCAACTTGTATA | 19 |
| | | | | TAGACGGTAAAATAAACACCAAGA | |
| | | | Reverse | TATGTCCGTCGTTGCGAAGTGCTGGACA | 20 |
| | | | | GTTACCTTATTCAAACATCA | |
| 8 | rs1884444 | 1: 67168129 | Forward | CCAAAGTCGTTAAGCTGCCAATTCCTGCT | 21 |
| | | | | TCCAGACATGAATCA | |
| | | | Reverse | TATGTCCGTCGTTGCGAAGTGCACCATAC | 22 |
| | | | | CTCCATGACACCA | |
| 9 | rs16754 | 11: 32396399 | Forward | CCAAAGTCGTTAAGCTGCCAACTCTCTGC | 23 |
| | | | | CTGCAGGATGTG | |
| | | | Reverse | TATGTCCGTCGTTGCGAAGTGGCGTTTCT | 24 |
| | | | | CACTGGTCTCAGAT | |
| 10 | rs28932178 | 5: 177210575 | Forward | CCAAAGTCGTTAAGCTGCCAAACTAAGA | 25 |
| | | | | GTGCAGAGCCTGGAA | |
| | | | Reverse | TATGTCCGTCGTTGCGAAGTGTGGGGTTT | 26 |
| | | | | GTGAACAAGAGTAGA | |
| 11 | rs10741037 | 10: 24041034 | Forward | CCAAAGTCGTTAAGCTGCCAACACTTTAT | 27 |
| | | | | CAGACACAGTTATGTGCT | |
| | | | Reverse | TATGTCCGTCGTTGCGAAGTGGCCCACAT | 28 |
| | | | | TTAGAATTTAGAGGTAACT | |
| 12 | rs10805227 | 4: 21513579 | Forward | CCAAAGTCGTTAAGCTGCCAACTATCTGC | 29 |
| | | | | AGGATTGTGTTCAATGTA | |
| | | | Reverse | TATGTCCGTCGTTGCGAAGTGAGCAAAC | 30 |
| | | | | CACTGGGGAAAATACT | |
| 13 | rs10833604 | 11: 21712549 | Forward | CCAAAGTCGTTAAGCTGCCAACTCTCTAG | 31 |
| | | | | AGTGCAGATTGGTAGAA | |
| | | | Reverse | TATGTCCGTCGTTGCGAAGTGCAAGTGA | 32 |
| | | | | GAGACCAAGGAAAACAA | |
| 14 | rs10964389 | 9: 20022427 | Forward | CCAAAGTCGTTAAGCTGCCAACAAAGTT | 33 |
| | | | | GATAAATTAAAGGACTAAGGCAC | |
| | | | Reverse | TATGTCCGTCGTTGCGAAGTGTTAAATCC | 34 |
| | | | | TGCCTCTACTACTTGCT | |
| 15 | rs11045749 | 12: 21092846 | Forward | CCAAAGTCGTTAAGCTGCCAACATTCTGT | 35 |
| | | | | CTGGGATGAGGTGAT | |
| | | | Reverse | TATGTCCGTCGTTGCGAAGTGAACAGTTA | 36 |
| | | | | TATGAAAAAAATGCTCAATATCACT | |
| 16 | rs12213948 | 6: 25424028 | Forward | CCAAAGTCGTTAAGCTGCCAATGAAAGA | 37 |
| | | | | CGTCACAGCAAGGT | |
| | | | Reverse | TATGTCCGTCGTTGCGAAGTGCGGGGAC | 38 |
| | | | | CAGGAGCAAAG | |
| 17 | rs12259813 | 10: 26967533 | Forward | CCAAAGTCGTTAAGCTGCCAATGTAGGA | 39 |
| | | | | GAGATTGGGCTAGAGAG | |
| | | | Reverse | TATGTCCGTCGTTGCGAAGTGCTCTACCT | 40 |
| | | | | GGGAAATCTCATTCATTC | |
| 18 | rs1516755 | 11: 21061449 | Forward | CCAAAGTCGTTAAGCTGCCAACTAACTTC | 41 |
| | | | | CTAACTAAAACTTTACAGTGGA | |
| | | | Reverse | TATGTCCGTCGTTGCGAAGTGTCAGTAGA | 42 |
| | | | | ACTTTGAAGGGTACACA | |
| 19 | rs1937037 | 9: 22913892 | Forward | CCAAAGTCGTTAAGCTGCCAAGCACGTA | 43 |
| | | | | GATGAAATTGCCCCATA | |
| | | | Reverse | TATGTCCGTCGTTGCGAAGTGACTTCCTA | 44 |
| | | | | CTTAGCCCTTTAGAAATGTAA | |
| 20 | rs2616187 | 8: 20811932 | Forward | CCAAAGTCGTTAAGCTGCCAAGGAAAAT | 45 |
| | | | | ATGTCTAAAAAGGCTCTGGAG | |
| | | | Reverse | TATGTCCGTCGTTGCGAAGTGCCAGTGCA | 46 |
| | | | | GTGTTTCTCAAACTC | |
| 21 | rs2710998 | 7: 24953942 | Forward | CCAAAGTCGTTAAGCTGCCAAGTTTGTTC | 47 |
| | | | | TAAGGTTCATCTGGTGAT | |
| | | | Reverse | TATGTCCGTCGTTGCGAAGTGGCTCATGA | 48 |
| | | | | AGAAAATAATCCTTATGGTAATC | |
| 22 | rs2874755 | 8: 27010387 | Forward | CCAAAGTCGTTAAGCTGCCAATGTCCCA | 49 |
| | | | | CTTTTTACCTCCCTTC | |
| | | | Reverse | TATGTCCGTCGTTGCGAAGTGCTTCATGG | 50 |
| | | | | AGGAGATAGTAACTAAGGT | |
| 23 | rs4665582 | 2: 23290086 | Forward | CCAAAGTCGTTAAGCTGCCAATGTGCTA | 51 |
| | | | | CGACAGAGCTAAGTAC | |
| | | | Reverse | TATGTCCGTCGTTGCGAAGTGTGGTCAGC | 52 |
| | | | | TTAAATAGCTACTGCT | |
| 24 | rs4712476 | 6: 20291800 | Forward | CCAAAGTCGTTAAGCTGCCAACCCCGGA | 53 |
| | | | | TGTCAGGGAATG | |
| | | | Reverse | TATGTCCGTCGTTGCGAAGTGGTGAGTAT | 54 |
| | | | | GCACGTCTCCATCT | |
| 25 | rs611628 | 1: 41832653 | Forward | CCAAAGTCGTTAAGCTGCCAACCAGGCA | 55 |
| | | | | CCACTGCTTTGT | |
| | | | Reverse | TATGTCCGTCGTTGCGAAGTGGCAAGGG | 56 |
| | | | | GACAGAAATTTGCTTATC | |
| 26 | rs7893462 | 10: 27939936 | Forward | CCAAAGTCGTTAAGCTGCCAAACCTTGTC | 57 |
| | | | | AAGAACCTAAATAGTGAGAA | |
| | | | Reverse | TATGTCCGTCGTTGCGAAGTGTGGGAGA | 58 |
| | | | | GTTCACTGCACCT | |
| 27 | rs7902135 | 10: 25287563 | Forward | CCAAAGTCGTTAAGCTGCCAACGTGGGC | 59 |
| | | | | TAGTCAAGAATATAAAATGTTAG | |
| | | | Reverse | TATGTCCGTCGTTGCGAAGTGCATGCAG | 60 |
| | | | | GTGGTGTGAATCTC | |
| 28 | rs9466930 | 6: 23840904 | Forward | CCAAAGTCGTTAAGCTGCCAATGTGTGG | 61 |
| | | | | CTCAGTATACCACTTAG | |
| | | | Reverse | TATGTCCGTCGTTGCGAAGTGCTCAAGCC | 62 |
| | | | | ATGTCATATTTTCAAATAGAC | |
| 29 | rs2862909 | 13: 24527029 | Forward | CCAAAGTCGTTAAGCTGCCAAGCACATC | 63 |
| | | | | ATACATTATTTCTGTTGCTAT | |
| | | | Reverse | TATGTCCGTCGTTGCGAAGTGAGAGCCC | 64 |
| | | | | ACTTAGCATCTCCA | |
| 30 | rs1338945 | 20: 23119232 | Forward | CCAAAGTCGTTAAGCTGCCAAGAAATAT | 65 |
| | | | | TGCTGGGGTCAGCG | |
| | | | Reverse | TATGTCCGTCGTTGCGAAGTGGGAGGGT | 66 |
| | | | | TTAAGGTGTTTTATGTTTTG | |

TABLE 8
Position barcoding primer sequences

| Primer | | |
|---|---|---|
| Name | Primer Sequence (5′→3′) | SEQ ID NO: |
| FP-R1 | ATACGTGCCAAAGTCGTTAAGCTGCCAA | 67 |
| FP-R2 | TGAAGTTCCAAAGTCGTTAAGCTGCCAA | 68 |
| FP-R3 | TGATTAGCCAAAGTCGTTAAGCTGCCAA | 69 |
| FP-R4 | CTAATCACCAAAGTCGTTAAGCTGCCAA | 70 |
| FP-R5 | ATGACGCCCAAAGTCGTTAAGCTGCCAA | 71 |
| FP-R6 | GTAATGTCCAAAGTCGTTAAGCTGCCAA | 72 |
| FP-R7 | AAACTTCCCAAAGTCGTTAAGCTGCCAA | 73 |
| FP-R8 | TCCGAGACCAAAGTCGTTAAGCTGCCAA | 74 |
| RP-C1 | GTCAATCTATGTCCGTCGTTGCGAAGTG | 75 |
| RP-C2 | CGCTATGTATGTCCGTCGTTGCGAAGTG | 76 |
| RP-C3 | ACCGATTTATGTCCGTCGTTGCGAAGTG | 77 |
| RP-C4 | AACACCGTATGTCCGTCGTTGCGAAGTG | 78 |
| RP-C5 | CTCGGAATATGTCCGTCGTTGCGAAGTG | 79 |
| RP-C6 | GACTGATTATGTCCGTCGTTGCGAAGTG | 80 |
| RP-C7 | TACTGCGTATGTCCGTCGTTGCGAAGTG | 81 |
| RP-C8 | AACTGGCTATGTCCGTCGTTGCGAAGTG | 82 |
| RP-C9 | ATCGGTCTATGTCCGTCGTTGCGAAGTG | 83 |
| RP-C10 | GCCAGTTTATGTCCGTCGTTGCGAAGTG | 84 |
| RP-C11 | CACGTATTATGTCCGTCGTTGCGAAGTG | 85 |
| RP-C12 | CGCATGTTATGTCCGTCGTTGCGAAGTG | 86 |

Single donor sample analysis. Encapsulating materials were barcoded by the co-encapsulated HUVEC donor cells, and thus determining HUVEC donor identities through sequencing data analysis could reveal material information. NGS fastq data was demultiplexed by row and column barcodes to re-group sequences amplified from the same DNA input. Sequence alignment to target amplicons was conducted in bowtie2. Then for each amplicon sequence, grep function was applied to search the dominant and variant alleles to calculate variant allele frequency (VAF) for each SNIP locus. Since capsules implanted in mice contained only one HUVEC donor, the HUVEC donor with the highest match rate to the analyte sample was identified as the barcoding cell.

Dual donors sample analysis: Log-likelihood was employed to analyze explanted samples that encapsulated one or two HUVEC donors. Specifically, SNP VAF profiles were calculated for all possible compositions of donor cells. VAF_{i,j,k} in equation (1) represents the expected VAF of the kth SNP when donor i and donor j were mixed at a ratio of 1:R. Here, all possible combinations of 20 different HUVEC donors had 20×20=400 different compositions. For each possible composition, the observed VAF of each SNP, depending on how close or far the observed value was from the VAF in the composition, a probability, p(i. j, k), was calculated from Gaussian distribution. The overall log-likelihood of each composition, Log(L_{i,j}), is obtained from summing the log-likelihood of all SNPs, and the composition with the highest overall log-likelihood is determined as the barcoding cell composition.

$${VAF}_{i , j , k} = \frac{1}{1 + R} \cdot {VAF}_{i , k} + \frac{κ}{1 + R} \cdot {VAF}_{j , k} \qquad ( 1 )$$

$${L}_{i , j} = {\prod }_{k = 1}^{30} p ( i , j , k ) \qquad ( 2 )$$

$$Log ( {L}_{i , j} ) = {\prod }_{k = 1}^{30} log [ p ( i , j , k ) ] \qquad ( 3 )$$

Statistical analysis. Statistical analysis for material screening used a customized MATLAB script. The retrieved percentage for good materials was average among biological replicates (the number of mice N=5). And an error bar of 95% confidence interval was calculated for each material from binomial distribution. Any two materials with non-overlapping error bar were identified as statistically significant (p<0.05). For statistical analysis of other graphs, one-way or two-way ANOVA with Bonferroni multiple-comparison correction was used (****P<0.0001, ***P<0.002).

### C. Results and Discussion

Material development, method development for NGS screening and screening in immune-compromised mice. In order to screen a large library of immunoprotective hydrogels in vivo using cellular barcoding, a set of successive methods were developed for identifying a cell encapsulation material following in vivo screening of cell encapsulation materials, the methods comprising (a) preparing a plurality of barcode cells derived from one or more subjects, wherein each composition of barcode cells comprises a unique profile of a plurality of SNPs that serve as a genetic barcode for each cell encapsulation material; (b) fabricating capsules using various encapsulation materials and barcode cells; (c) implanting the capsules in vivo into a test subject; (d) explanting the capsules after a set period of time; and (d) determining the sequence of the plurality of SNPs in the barcode cells of each explanted capsule, thereby identifying the cell encapsulation material of each capsule (FIG. 2A) Earlier published reports demonstrated the development of a 774 combinatorically synthesized hydrogel library and the identification of three lead triazole containing anti-fibrotic covalently modified alginate analogues with similar molecular structure (1). Careful structural analysis showed that the commonality between the lead analogues were in the presence of triazole (a heterocyclic five-membered ring with two carbon atoms and three nitrogen atoms). This suggests that triazole containing molecules may regulate immune cell populations at the surface of these biomaterials, including macrophages, inhibiting their activation and interrupting the fibrotic processes. In this present manuscript, a total of 211 new alginate analogs (encapsulation materials) were synthesized by triazole surface modification (FIG. 1). A combinatorial biomaterial approach was developed to generate a library of alginate-based hydrogels by covalently attached small molecules functionalities by keeping the triazole analogues common to all. Low molecular weight (MW), ultrapure alginate UPVLVG with high guluronate (G) content (>60% G, ˜25 kDa MW, NovaMatrix) was used as the starting material. Three hydrophilic PEG based linkers (FIG. 1, Table 2) were used to create 150 unique polymers. Two hydrophobic linkers were used to generate another 61 unique polymers all containing triazole (FIG. 1, Table 2). Further the covalently attached characterized using elemental analysis, nuclear magnetic resonance spectroscopy (NMR), and gelation assays (FIGS. 3D, 3E, and 3A, respectively). After initial characterization studies including purity, solubility and gel forming ability about 149 alginate analogues were selected for in vivo screening purposes (Table 3). Further various in vitro characterizations of newly formed capsules with or without cells were carried out using 1) live-dead assays using fluorescent based imaging of human umbilical vein endothelial cells (HUVECs) as well as 2) bright-field and dark-field microscopy to verify homogenous shape and size for all capsules. Twenty unique donors of HUVECs are sequenced via next generation sequencing (NGS) and a unique bar-coding has been established to identify their individual single nucleotide polymorphisms (SNPs) which was used as a barcode to tag and identify different encapsulation materials. Successful DNA extraction were done from in vitro and in vivo explanted capsules with high quality and enough human DNA for polymerase chain reaction (PCR) amplification followed by NGS for human SNP identification. Deep sequencing of HUVEC cells revealed genetic profiles of the selected 30 non-pathogenic SNPs. Each SNP locus could possess one of the three genotypes: homozygous for wildtype (WT) allele, heterozygous, or homozygous for variant allele. The genotypes of the 30 SNPs altogether formed unique genetic profiles that could ambiguously represent the HUVEC cell identify (FIG. 2B). FIG. 2C is a representative image from the screening (pre implant and post explant). Thus, by encapsulating a single donor HUVEC in each different hydrogel material, the material identity is uniquely tagged by cellular barcodes that could be read through sequencing post-implantation. The extraction method of gDNA from a single capsule was optimized (FIG. 4) to increase DNA input for sequencing. Further, the workflow was established for NGS library preparation and material identifications (FIG. 5 and FIG. 6).

To demonstrate that all donors (H1-H20, Table 3) can be deconvoluted by NGS, a mixture of 20 different hydrogel capsules (made from the library of 211 novel hydrogel analogs) encapsulating 20 unique HUVECs were implanted in the intraperitoneal (IP) space of NSG (NOD SCID gamma) mice for four weeks. The NSG mouse lacks mature T cells, B cells, and natural killer cells to induce normal innate immunity function. Screening in immunodeficient mice allows us to assess this cellular barcoding strategy with minimal host immune responses against implanted materials. Imaging of post-retrieval capsules showed minimal cell deposition, indicating a lack of fibrosis and high viability of encapsulated cells (FIG. 2C). The capsules from one mouse were analyzed with the NGS technique, and 195 out of 200 capsules (˜96.5%) were successfully identified based on their unique cellular barcoding (FIG. 2D). The identified percentages were evenly distributed across donors. These results indicated that cellular barcoding using different HUVEC donors and NGS genotyping can be leveraged as a suitable strategy for determining biomaterial identity without altering materials properties.

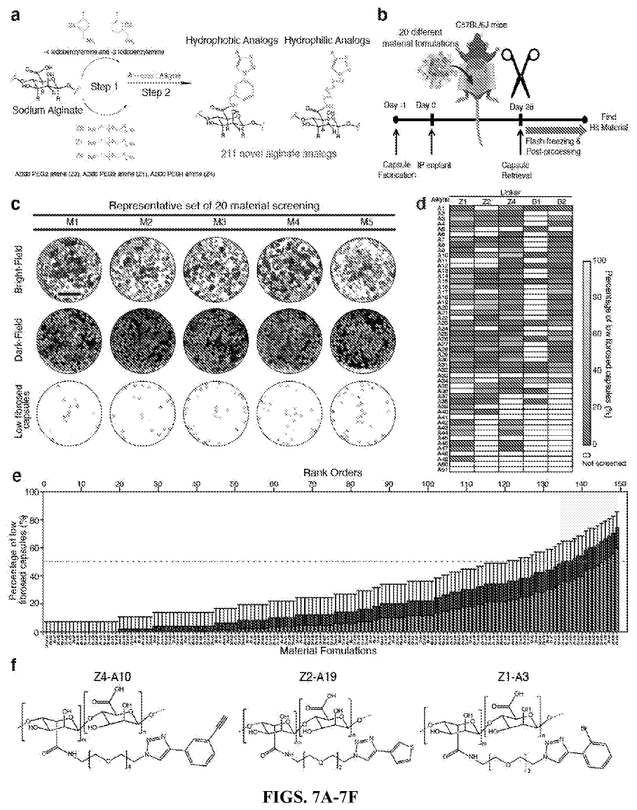

Material Screening in Immune-competent Mice. To measure levels of immune responses, these newly synthesized alginate analogs were evaluated using a novel high throughput in vivo screening method (FIG. 7A). The unique single nucleotide polymorphism (SNP) genotypes of the 20 different HUVEC donors were used to barcode each material. A mixture of 20 different materials (10 capsules per material) was implanted into intraperitoneal (IP) space in each mouse for each round. A total of ˜150 novel materials were screened using this method. After 28 days of in vivo implantation in mice, the capsules were retrieved from IP space, and post processes were performed to identify the material's donor pairs (FIG. 7B). Those capsules which are showing low fibrotic responses suggests, without being bound by theory, that the materials have immune-protective properties. Since these explanted capsules were a mixture of 20 different materials, the encapsulated HUVEC donors should be identified using a high-throughput method to find corresponding materials. All explanted capsules were separated into three groups (L: Low, M: Medium, H: High fibrosed capsules) based on surface fibrosis levels under the microscope. Capsules in the low fibrosed (L) groups showed a clear surface with low fibrosis deposition. In contrast, the high fibrosed (H) group showed higher deposition of fibrotic tissues on capsules surface, resulting in the formation of aggregate due to fibrotic overgrowth between capsules. Capsules between two groups were assigned as medium fibrosed group (M). Only the L group capsules were selected for materials identification using NGS assay (FIG. 7C).

To increase the throughput of material screening, the workflow was designed to process a batch of 96 extracted DNA samples in a run. Following amplifying SNP amplicons on a 96-well PCR plate, each sample on the plate would be position-barcoded by a unique combination of forward and reverse primers containing Hamming barcodes to represent their positions on the plate. This allows that the barcoded amplicons could be pooled without losing sample information, and the pooled library could then be processed for sequencing on Illumina platform with ligation-based method. For in-vivo screening in mice, two of such libraries were able to screen explanted capsuled from one animal, and the estimated sequencing space would be approximately 11.5 million reads (based on 2000 coverage/plex×30 plex×96 samples/library), or 2.9% of capacity of a NextSeq flow cell.

All identified capsules were plotted (FIG. 7E) to find the hit materials with anti-inflammatory properties. The percentage of low fibrosed capsules represented the material's anti-fibrosis performance (FIG. 7E, Table 3). For each round, 10 capsules were implanted per material, and so the graph was plotted as a percentage of low fibrosed capsules (compare with implanted capsules number). It was found that a total of 15 alginate analogs (blue and orange color bars in FIG. 7E), (including one previously known material, Z1-A34, marked as the green bar in FIG. 7E), showed above 50% low fibrosed capsules, which means 5 capsules on average came out clearly from 10 implanted capsules. The screening result also was plotted as a heat map (FIG. 7D) to visualize the fibrosis outcomes of different combinations of alkyne and linkers. These results show that some of the alkynes (A3, A17, A19, A30, A43) displayed low fibrosis levels across the different linkers. These alkynes contain phenyl bromine, pyridine, thiophene, ethoxy, and cyclopropyl functionality, respectively (Table 2). Some similarities of these alkynes include a lack of branched structures and longer carbon chains, which makes these alkynes relatively compact. Additionally, these alkynes all contain a carbon ring structure, and most have an electronegative atom (O, N, S, Br) in or around the carbon ring.

FIG. 7F shows the chemical structures of top three identified lead alginate analogues (orange bars in FIG. 7E) with lowest FBR or fibrosis (Z4-A10, Z2-A19, and Z1-A3). All the three new analogues contain triazole moiety in the backbone that further supports the role of triazole towards prohibiting fibrosis. Interestingly all the three lead materials have hydrophilic peg linkers attached with the alginate, in addition of the functionalization of bromobenzene (Z1-A3), thiophene (Z2-A19) and ethynylbenzene (Z4-A10) to the triazole (FIG. 7F). Notably, in the top 15 leads, only two hydrophobic analogs were identified (B2-A17 and B1-A34), one of which was later used for catheter coating application.

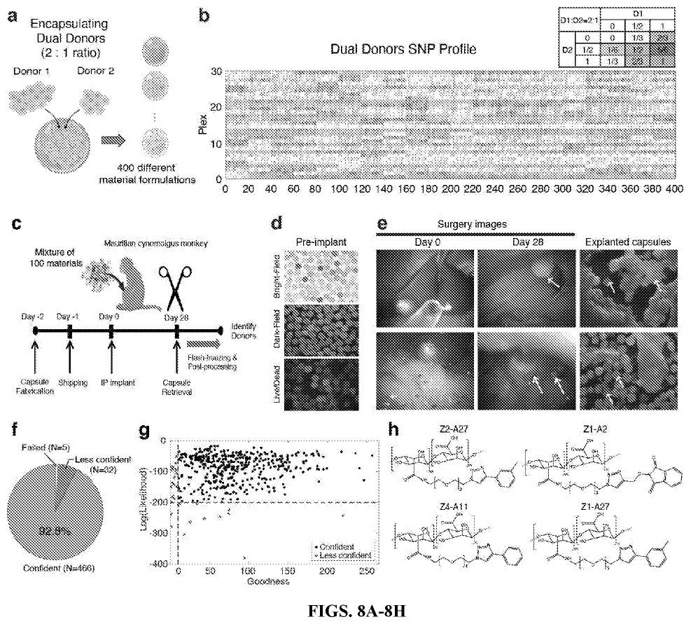

Dual donor cells barcoding expands the high-throughput potential. To translate this novel barcoding strategy to screen a larger batch of anti-fibrotic biomaterials, a dual donor barcoding strategy was utilized, and its feasibility was tested in an NHP model. Only 20 different materials were implanted in one mouse. In contrast, in the NHP model, due to the larger capacity of implantable space, the material batch size can be increased to 100, resulting in a 5-fold increase in throughput. With single donor encapsulation, its throughput is limited by the number of available unique donors. Here, a new method using a dual donor barcoding strategy was devised to increase the screening throughput without purchasing and validating new cell donors. By mixing two different HUVECs at a ratio of 1:2 (FIG. 8A), 400 unique permutations of barcoding compositions were created with only 20 HUVEC donors (FIG. 8B), which significantly expands existing donors' barcoding capacity. With this ratio, the convoluted variant allele frequencies (VAFs) now include seven possibilities (0, ⅙, ⅓, ½, ⅔, ⅚, and 1). Hence, the donor identification strategy is modified from direct comparison with individual donors to log-likelihood analysis (FIG. 6). The likelihoods of all possible combinations were assessed from Gaussian distribution to determine the mixed donor identity. The mixing ratio of two donors was optimized (FIG. 9), and small cohort screening (comparison of three materials) was performed to confirm the feasibility of a dual donor barcoding strategy (FIG. 10).

Following the development of 400 novel dual barcodes, a set of 100 unique triazole containing alginate analogs (randomly selected from the 211 newly synthesized analogs library (represented in FIG. 1) encapsulated with the unique combinations of two different HUVECs (1:2 ratio) were implanted (30 capsules per materials) in the IP space of an NHP for four weeks (FIG. 5C). The representative images of pre-implant capsules showed the homogenous distribution of 1.5 mm capsules (FIG. 8D). Live/dead staining of pre-implant capsules confirms that cells were viable before implantation to NHP (FIG. 8D). After four weeks, all the free-floating capsules in IP space were retrieved and used for material identification (white arrow: tissue aggregated capsules, yellow arrow: free-floating capsules, FIG. 8E) using NGS assay. From 3000 implanted capsules, a total 503 free-floating capsules were retrieved and used for NGS analysis, resulting in ˜92.6% of sampled capsules being successfully identified with high confidence (FIG. 8F). The remaining 7.4% capsules were not confidently identified either because of insufficient sequencing depth (N=5, failed) or low barcode matching likelihood (N=32, less confident). The distribution of capsules in confidence space was plotted in FIG. 8G. This study successfully demonstrates the in vivo screening applicability and feasibility of 100 new materials in the NHP model with dual barcoding strategies. Four lead hydrogels were identified from 100's that showed minimum fibrosis in NHP (Z1-A2, Z4-A11, Z1-A27, and Z2-A27, FIG. 8H). All these four lead hydrogels contain hydrophilic linkers. Interestingly, these four leads from NHP screening were not identified as top 15 leads in the mouse study and hence were not utilized for further applications in this study.

New Lead Materials Screened from Mice Study Mitigated Foreign Body Responses and can be Used for Various Coating Application to Protect from Immune Response.

Chemically modified alginates reduce FBRs in immune-competent mice in individual settings. All materials used for screening were tested in mixed conditions for high-throughput assay. Therefore, it was desired to confirm whether the screened top lead materials have immune-protective property in individual setting. Microcapsules with 300˜400 μm size were tested for implantation for 2 weeks since the smaller size of capsules can provoke immune responses in a short period. The top ten lead materials, one positive control hydrogel reported earlier (Z1-A34), and one negative control hydrogel (SLG20) were used for this study. After 2 weeks of implantation in IP space in mice, dark-field images showed less fibrosis deposition in lead materials than controls (FIG. 11A). The SLG20 control had more elevated immune responses, and most microcapsules were aggregated within fibrosis tissues, in contrast to selected lead materials, including Z4-A10 and Z1-A3. Surface fibrosis levels were determined by imaging capsules with macrophage and fibroblast markers and RT-qPCR analysis (FIG. 11B-11C) 6.50. From immunofluorescence imaging (FIG. 11B), Z4-A10 and Z1-A3 showed the least intensity of macrophage (CD68, green) and myofibroblast (α-SMA, red) markers compared with SLG20 control, indicating low fibrosis levels. Reverse transcription PCR analysis of fibrotic markers (α-SMA and Col1a1) revealed that lead materials have significantly lower expression of both markers, indicating lower fibrosis and reduced collagen deposition on capsule surface compared to SLG20 (FIG. 11C). Z4-A10 and Z1-A3 looked most promising in preventing the FBRs among all top leads and hence were considered for further applications, including delivery of xenogeneic human islets (Z4-A10) in diabetic rodents and coating of medical-grade catheters (Z1-A3). These results confirmed the anti-fibrosis effect of screened lead materials in individual settings, providing various immunomodulatory application opportunities.

### Lead Hydrogel Restores Long-Term Glycemia Using Xenogeneic Human Islets in an Immunocompetent Animal Model

Anti-fibrotic alginate (Z4-A10, Schemes 5 and 6) hydrogels of the present invention were used to encapsulate xenogeneic human islets. These formulations provide a highly porous and anti-fibrotic hydrogel outer membrane to enable long-term nutrient diffusion, high islet viability, and low fibrosis in vivo. High-throughput screening in mice used a high density of cell loading, ˜30,000 HUVECs per capsule, to provide enhanced selection pressure for identifying materials that can protect densely packed encapsulated xenogeneic cells from rejection. For the diabetes correction studies described herein, a similar cell density per capsule (15K-60K cells per capsule) was maintained to assess the efficacy of lead formulation in enabling long-lasting viability and protection of pancreatic islets in STZ induced C57BL/6J mice.

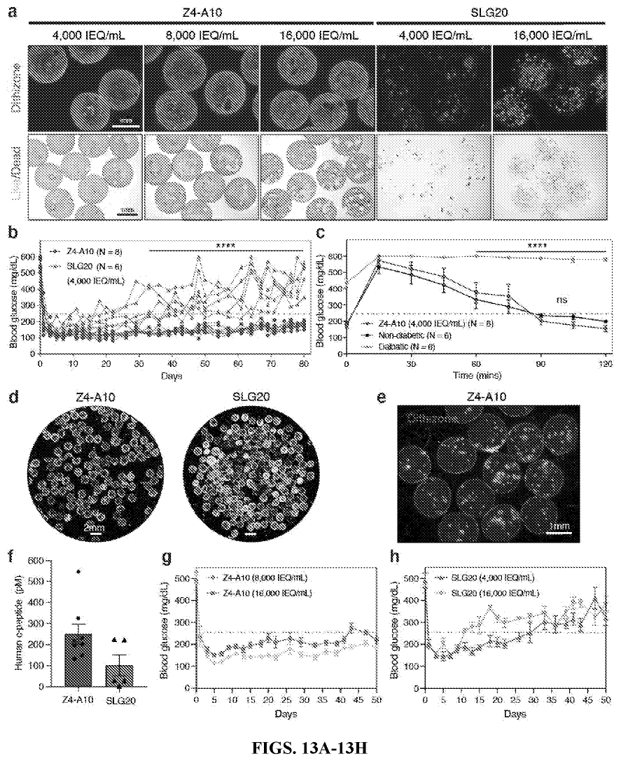

Capsules with three different densities (4K IEQ/mL of alginate, 8K IEQ/mL of alginate, and 16K IEQ/mL of alginates, FIG. 12) of human islets were prepared using Z4-A10 alginates, the lead triazole containing alginate identified through the high-throughput screening (FIG. 7E-7F). Control SLG20 capsules were prepared at islet cell densities of 4K IEQ/mL and 16K IEQ/mL. The pre-implant dithizone staining and live/dead imaging of the capsule groups (Z4-A10 and control SLG20) demonstrated the viability of the islets (FIG. 13A). The Z4-A10 capsules at a density of 4K IEQ/mL demonstrated long-term restoration of euglycemia and maintain glycemic correction until 80 days of data recording with the average blood glucose (BG) levels below 250, considered as the BG level of a healthy mouse, at a fasting condition (FIG. 13B). However, the control SLG20 alginate at the same dose (IEQ density of 4K/mL) failed to maintain glycemic correction for more than four weeks. Intravenous glucose tolerance test (IVGTT) was performed after four hours of fasting on day 75, showing encapsulated islet cells restored normoglycemia to a rate comparable with healthy C57BL/6J mice (FIG. 13C). Further, the postretrieval capsule images (dark-field) displayed minimal fibrotic overgrowth on the surface of Z4-A10 capsules compared with SLG20 (FIG. 13D). Dithizone staining also supports the long-term islet viability after 80 days of implantation (FIG. 13E). The concentration of human c-peptide, a surrogate biomarker for insulin production, was measured from the serum separated from mouse blood 80 days post-transplantation. Higher levels of c-peptide secretion were observed in the Z4-A10 group compared to SLG20, suggesting, without being bound by theory, better improved long-term viability (FIG. 13F).

In high-density encapsulation groups, Z4-A10 capsules with 16K IEQ/mL concentration could maintain long-term glycemic control >50 days of function (FIG. 13G). In contrast, the control SLG20 group failed to maintain glycemic control for no more than 10 days of implantation (FIG. 13H). These results suggest, again without being bound by theory, that Z4-A10 is enhanced in the ability to protect encapsulated islets from foreign body response, which maintains longer viability and function of grafts.

Lead anti-fibrotic materials show low fibrosis when coating catheters. Towards testing the effectiveness of newly developed small molecules against preventing fibrosis in the context of other medical devices, medical grade silicone catheters were plasma treated and coated with methacrylol modified Z1-A3 (one new hydrophilic lead) and B2-A17 (one new hydrophobic lead) (FIG. 14A, Scheme 7-10). Silicone catheters are inherently hydrophobic in nature. Hence, one top hydrophobic lead (B2-A17) and one hydrophilic lead (Z1-A3) were selected to determine if hydrophilicity/hydrophobicity affects the mitigation of fibrosis. XPS and ToF-SIMS were used to analyze the surface chemistry of the unmodified and coated catheters to confirm the successful coating of the catheters (FIG. 14B-14C, FIGS. 14A-14C).

To test the fibrotic response on uncoated and small molecule coated catheters in a pro-fibrotic C57BL/6J mice model, these catheters were implanted in mice subcutaneous space for four weeks. Histology and imaging of the explanted catheters after four weeks showed that the unmodified catheter explant led to thicker dark purple tissue deposition (which is collagen-rich and indicative of fibrotic overgrowth23) between the catheter and the skin tissue compared to the small molecules coated catheters (Met-Z1-A3 and Met-B2-A17) (FIG. 14D-14E, FIG. 15D-15E). The results indicated that, without being bound by theory, the small molecule coated catheters are more capable of preventing FBR than the unmodified catheters. It is also noticeable that the hydrophobic lead (Met-B2-A17) performed better in preventing fibrotic deposition than one of the hydrophilic leads (Met-Z1-A3) when used to coat hydrophobic silicone catheters23.

### NMR Data for the Top 20 Better Performing Alginate Analogues.

Z1A3: ¹H (600 MHz; D₂O): 2.99 (4H, s, N—CH₂—CH₂—S), 3.20-3.53 (m, alginate protons), 3.46 (4H, s, N—CH₂—CH₂—S), 3.52-3.76 (16H, m, ethoxy), 3.7-5.2 (m, alginate protons), 8.21 (1H, s, triazole).
Z1A5: ¹H (600 MHz; D₂O): 3.02 (4H, s, N—CH₂—CH₂—S), 3.25-3.57 (m, alginate protons), 3.52 (4H, s, N—CH₂—CH₂—S), 3.55-3.78 (16H, m, ethoxy), 3.8-5.3 (m, alginate protons), 8.48 (1H, s, triazole).
Z1A7: ¹H (600 MHz; D₂O): 2.95 (4H, s, N—CH₂—CH₂—S), 3.17-3.51 (m, alginate protons), 3.43 (4H, s, N—CH₂—CH₂—S), 3.52-3.73 (16H, m, ethoxy), 3.7-5.3 (m, alginate protons), 8.25 (1H, s, triazole).
Z1A14: ¹H (600 MHz; D₂O): 3.05 (4H, s, N—CH₂—CH₂—S), 3.20-3.45 (m, alginate protons), 3.43 (4H, s, N—CH₂—CH₂—S), 3.48-3.69 (16H, m, ethoxy), 3.73-5.3 (m, alginate protons), 8.31 (1H, s, triazole).
Z1A16: ¹H (600 MHz; D₂O): 2.92 (4H, s, N—CH₂—CH₂—S), 3.20-3.55 (m, alginate protons), 3.45 (4H, s, N—CH₂—CH₂—S), 3.52-3.74 (16H, m, ethoxy), 3.75-5.2 (m, alginate protons), 8.22 (1H, s, triazole).
Z1A19: ¹H (600 MHz; D₂O): 2.95 (4H, s, N—CH₂—CH₂—S), 3.15-3.50 (m, alginate protons), 3.48 (4H, s, N—CH₂—CH₂—S), 3.50-3.72 (16H, m, ethoxy), 3.70-5.3 (m, alginate protons), 8.20 (1H, s, triazole).
Z1A43: ¹H (600 MHz; D₂O): 2.95 (4H, s, N—CH₂—CH₂—S), 3.12-3.43 (m, alginate protons), 3.41 (4H, s, N—CH₂—CH₂—S), 3.45-3.68 (16H, m, ethoxy), 3.70-5.5 (m, alginate protons), 8.17 (1H, s, triazole).
Z2A19: ¹H (600 MHz; D₂O): 2.97 (4H, s, N—CH₂—CH₂—S), 3.20-3.56 (m, alginate protons), 3.53 (4H, s, N—CH₂—CH₂—S), 3.57-3.78 (16H, m, ethoxy), 3.80-5.6 (m, alginate protons), 8.24 (1H, s, triazole).
Z2A20: ¹H (600 MHz; D₂O): 2.95 (4H, s, N—CH₂—CH₂—S), 3.22-3.57 (m, alginate protons), 3.55 (4H, s, N—CH₂—CH₂—S), 3.58-3.82 (16H, m, ethoxy), 3.84-5.5 (m, alginate protons), 8.25 (1H, s, triazole).
Z2A28: ¹H (600 MHz; D₂O): 2.98 (4H, s, N—CH₂—CH₂—S), 3.24-3.58 (m, alginate protons), 3.56 (4H, s, N—CH₂—CH₂—S), 3.60-3.84 (16H, m, ethoxy), 3.82-5.4 (m, alginate protons), 8.28 (1H, s, triazole).
Z3A10: ¹H (600 MHz; D₂O): 3.03 (4H, s, N—CH₂—CH₂—S), 3.20-3.54 (m, alginate protons), 3.51 (4H, s, N—CH₂—CH₂—S), 3.54-3.82 (16H, m, ethoxy), 3.78-5.3 (m, alginate protons), 8.38 (1H, s, triazole).
Z3A22: ¹H (600 MHz; D₂O): 2.74 (4H, s, N—CH₂—CH₂—S), 3.05-3.38 (m, alginate protons), 3.34 (4H, s, N—CH₂—CH₂—S), 3.43-3.75 (16H, m, ethoxy), 3.78-5.2 (m, alginate protons), 8.08 (1H, s, triazole).
Z3A26: ¹H (600 MHz; D₂O): 2.98 (4H, s, N—CH₂—CH₂—S), 3.15-3.48 (m, alginate protons), 3.45 (4H, s, N—CH₂—CH₂—S), 3.52-3.76 (16H, m, ethoxy), 3.78-5.1 (m, alginate protons), 8.17 (1H, s, triazole).
Z3A27: ¹H (600 MHz; D₂O): 2.92 (4H, s, N—CH₂—CH₂—S), 3.08-3.46 (m, alginate protons), 3.41 (4H, s, N—CH₂—CH₂—S), 3.48-3.73 (16H, m, ethoxy), 3.76-5.7 (m, alginate protons), 8.35 (1H, s, triazole).
Z3A30: ¹H (600 MHz; D₂O): 2.95 (4H, s, N—CH₂—CH₂—S), 3.12-3.42 (m, alginate protons), 3.48 (4H, s, N—CH₂—CH₂—S), 3.46-3.82 (16H, m, ethoxy), 3.76-5.4 (m, alginate protons), 8.28 (1H, s, triazole).
Z3A43: ¹H (600 MHz; D₂O): 3.02 (4H, s, N—CH₂—CH₂—S), 3.08-3.43 (m, alginate protons), 3.48 (4H, s, N—CH₂—CH₂—S), 3.47-3.84 (16H, m, ethoxy), 3.73-5.6 (m, alginate protons), 8.32 (1H, s, triazole).
B1A34: ¹H (600 MHz; D₂O): 3.20-5.5 (m, alginate protons), 7.15-7.27 (1H, s, C—CH═C), 7.47-7.65 (1H, d, C—CH═C—N), 8.27 (1H, s, triazole).
B2A3: ¹H (600 MHz; D₂O): 3.07-5.7 (m, alginate protons), 6.82-7.07 (1H, s, C—CH═C), 7.17-7.38 (1H, d, C—CH═C—N), 7.75 (1H, s, triazole).
B2A17: ¹H (600 MHz; D₂O): 3.20-5.3 (m, alginate protons), 7.08-7.17 (1H, s, C—CH═C), 7.26-7.48 (1H, d, C—CH═C—N), 7.82 (1H, s, triazole).
B2A31: ¹H (600 MHz; D₂O): 3.08-5.5 (m, alginate protons), 7.03-7.18 (1H, s, C—CH═C), 7.25-7. (1H, d, C—CH═C—N), 7.74 (1H, s, triazole).

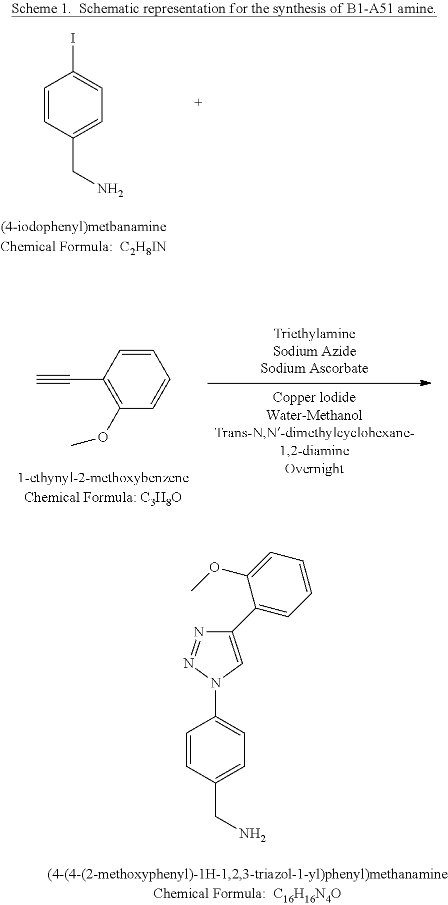

Mass and NMR data: ¹H (600 MHz; CDCl₃): 3.63 (s, 3H, CH₃—O—C), 4.01 (s, 2H, NH₂—CH₂—CH₂), 7.05 (s, 1H, —Obenzene), 7.17 (in, 2H, aromatic), 7.52 (in, 2H, aromatic), 7.79 (s, 1H, triazole) ¹³C (600 MHz; CDCl₃): 46.5 (NH₂CH₂), 56.8 (CH₃—O—CH₂), 111.4 (CH₃—O—CH₂—C(aromatic)), 121.6 (CH aromatic), 122.7 (CH triazole), 128.4 (CH aromatic), 135.8 (Cq-N aromatic), 144.9 (Cq-C aromatic), 146.9 (C triazole), 157.6 (0-C(aromatic)) ESI: M+1=281.1

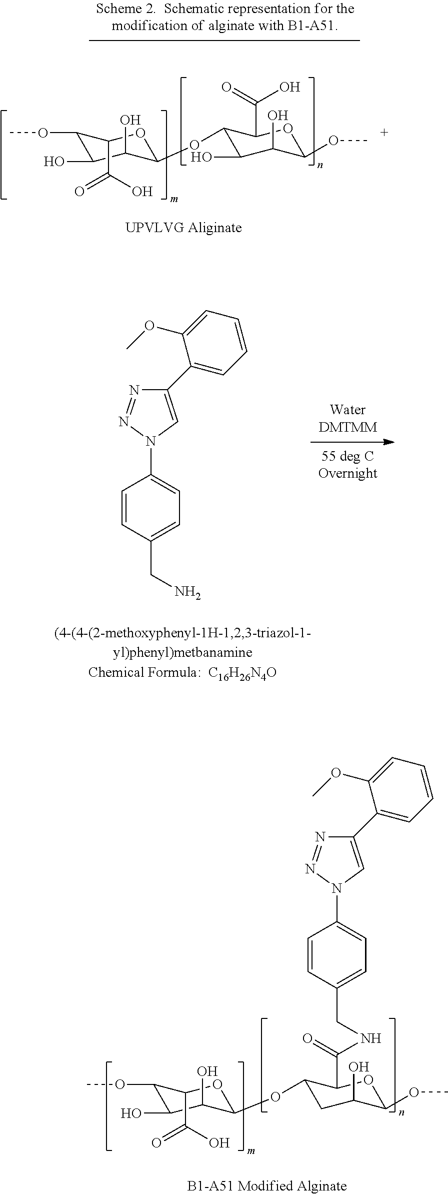

NMR data: 1 H (600 MHz; CDCl₃): 3.23-3.45 (m, alginate protons), 3.72 (s, 2H, C—H₂C—O), 3.75 (s, 3H, CH₃—O), 3.8-5.2 (m, alginate protons), 4.59 (s, 1H, C—CH—O), 4.61 (s, 2H, C—CH₂—O), 7.18 (s, 1H, O—CH aromatic), 7.32 (s, 2H, aromatic), 7.67 (s, 2H, aromatic), 8.04 (s, 1H, triazole).

### Elemental: C: 34.75%, H: 8.55%, N: 3.56%.

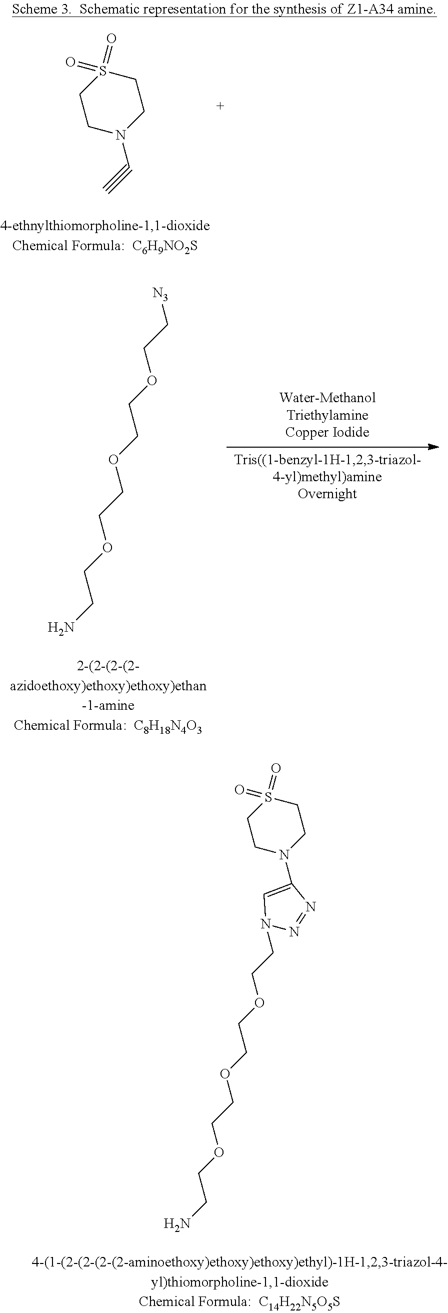

Mass and NMR data ¹H (600 MHz; CDCl₃): 2.87 (s, 2H, NH₂—CH₂—CH₂—O), 3.05 (m, 8H, N—CH₂—CH₂—S), 3.51 (t, 2H, NH₂—CH₂), 3.61 (m, 8H, PEG) 3.81 (s, 2H, —CH₂-Triazole), 3.89 (t, 2H, N—CH₂—CH₂—O), 4.55 (t, 2H, N—CH₂—CH₂—O), 7.51 (s, 2H, aromatic), 7.68 (s, 1H, triazole). ¹³C (600 MHz; CDCl₃): 41.74 (NH₂—CH₂), 50.42 (N—CH₂), 50.56 (N—CH₂ Thiomorpholine) 51.52 (S—CH₂ Thiomorpholine) 52.2 (Thiomorpholine-CH₂-Triazole), 69.54-73.14 (m, PEG), 124.10 (CH triazole), 143.32 (C triazole).

### ESI MS: [M+H+]=392.1939

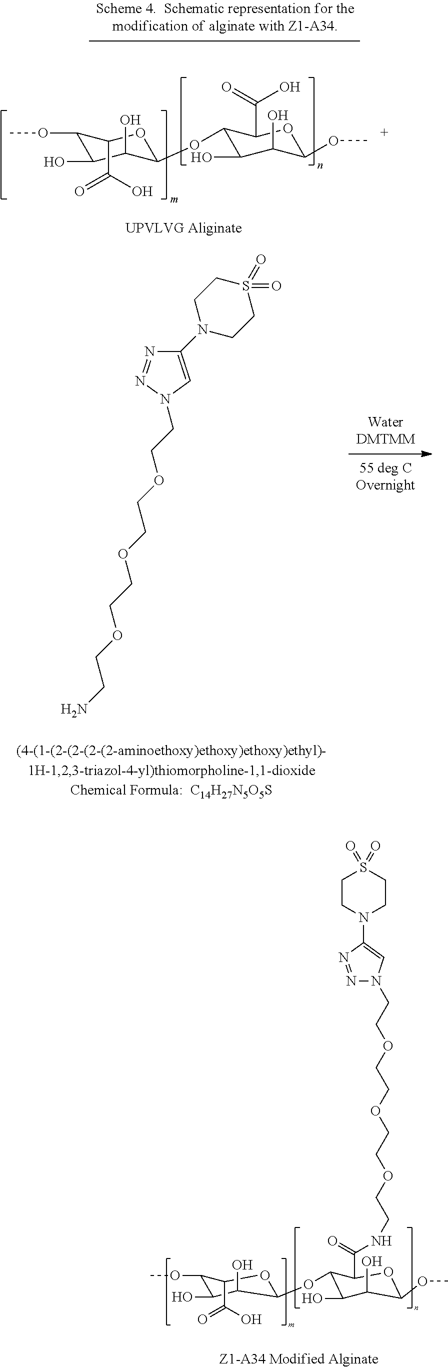

NMR data: 1 H (600 MHz; D₂O): 2.95 (s, 4H, N—CH₂—CH₂—S), 3.05-3.34 (m, alginate protons), 3.56 (s, 4H, N—CH₂—CH₂—S), 3.48-3.71 (16H, m, ethoxy), 3.65-5.32 (m, alginate protons), 8.08 (1H, s, triazole).

### Elemental: C: 35.67%, H: 4.34%, N: 5.08%, O: 33.50%.

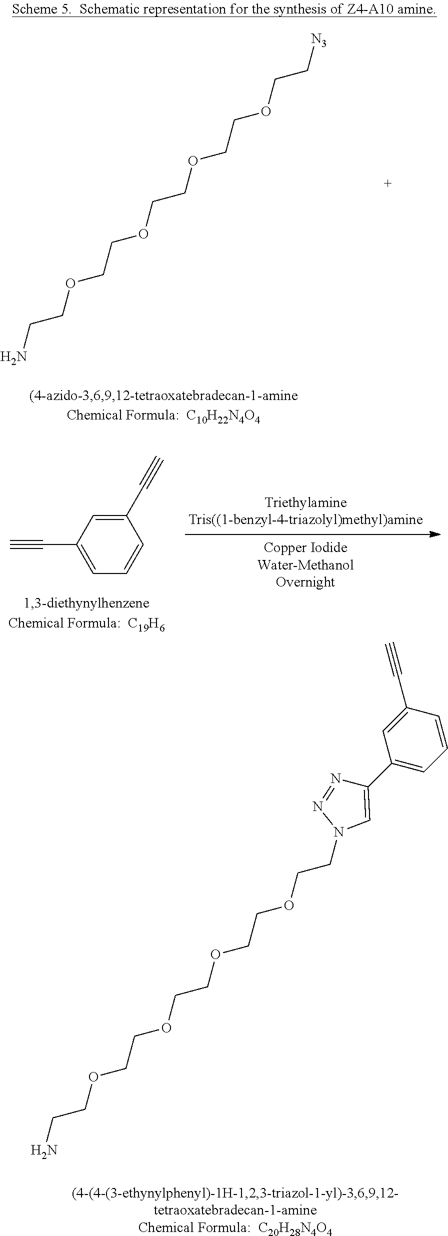

Mass and NMR data: ¹H (600 MHz; CDCl₃): 2.93 (s, 2H, NH₂—CH₂—CH₂—O), 3.15 (s, H, alkyne), 3.47 (t, 2H, NH₂—CH₂), 3.65 (m, 8H, PEG) 3.86 (s, 2H, CH₂-triazole), 3.89 (t, 2H, N—CH₂—CH₂—O), 4.55 (t, 2H, N—CH₂—CH₂—O), 7.69 (s, 1H, triazole), 7.79 (s, 2H, aromatic). ¹³C (600 MHz; CDCl₃): 40.7 (NH₂CH₂), 51.8 (N—CH₂—C), 69.2 (N—C—CH₂—O), 71.5 (C—CH₂—O), 73.2 (N—CH₂—CH₂—O), 82.1 (alkyne), 119.6 (H₂C═C), 122.6 (CH aromatic), 127.7 (CH aromatic), 129.5 (CH aromatic), 130.6 (CH aromatic), 132.4 (CH aromatic), 133.4 (C triazole), 139.7 (CH aromatic), 147.9 (1-ethylene), 166.5 (C, NH—C═O).

### ESI MS: [M+H+]=389.2080

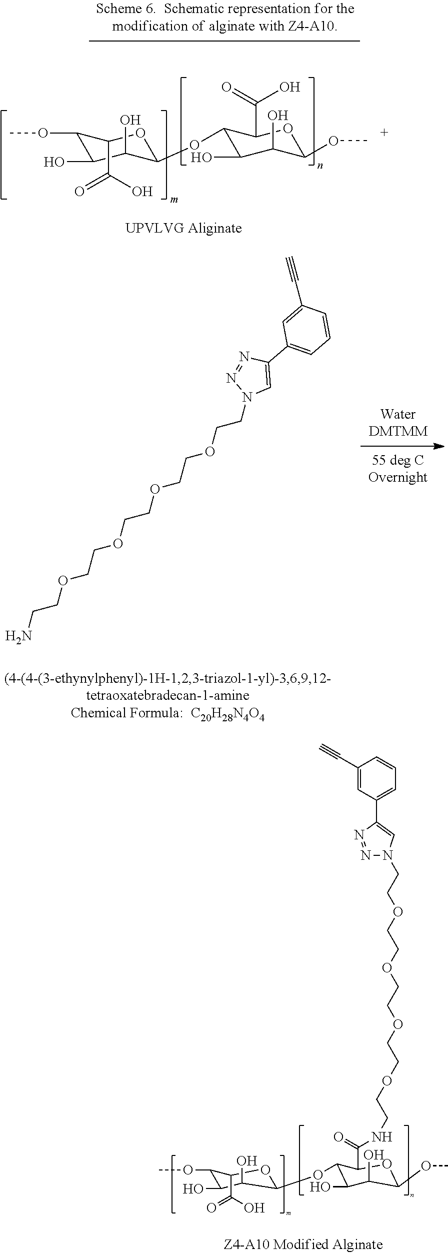

NMR: ¹H (600 MHz; D₂O): 3.12 (s, alkyne), 3.17-3.40 (m, alginate protons), 3.50-3.70 (m, 16H, ethoxy), 3.7-5.2 (m, alginate protons), 8.08 (s, 1H, triazole)

### Elemental: C: 40.45%, H: 5.60%, N: 6.22%.

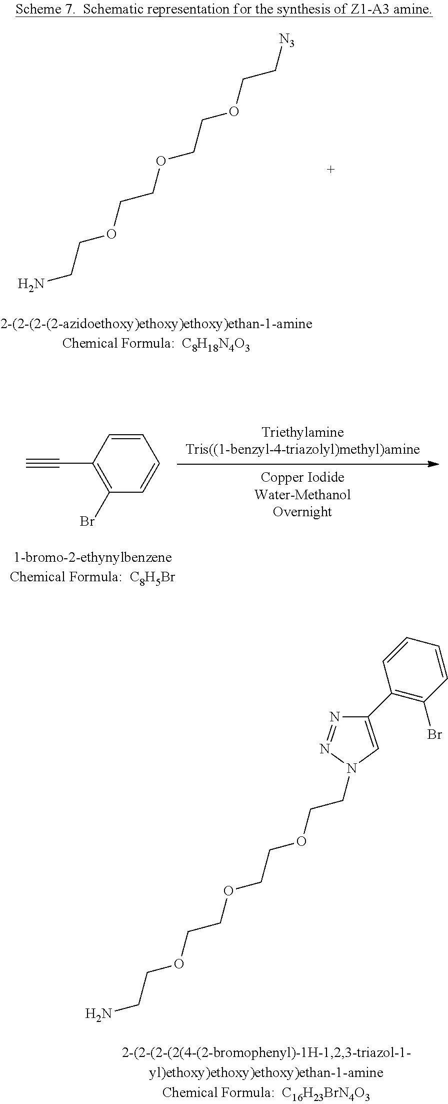

Mass and NMR data. ¹H (600 MHz; D₂O): 3.48 (m, 1H, methylene), 3.54 (m, 1H, methylene), 3.91 (s, 1H, NH₂—CH₂), 4.74 (d, 1H, J=12 Hz, O—CH₂-triazole), 4.89 (d, 1H, J=12 Hz, O—CH₂-triazole), 7.32 (m, 2H, aromatic), 7.54 (m, 2H, aromatic), 7.75 (m, 2H, aromatic), 8.46 (s, 1H, triazole). ¹³C (600 MHz; D₂O): 40.1 (NH₂CH₂), 49.9 (N—CH₂—C), 67.5 (N—C—CH₂—O), 70.1 (C—CH₂—O), 72 (N—CH₂—CH₂—O), 122.2 (CH aromatic), 127.2 (CH aromatic), 130.4 (CH aromatic), 134.1 (C triazole). ESI: M+1=399.1.

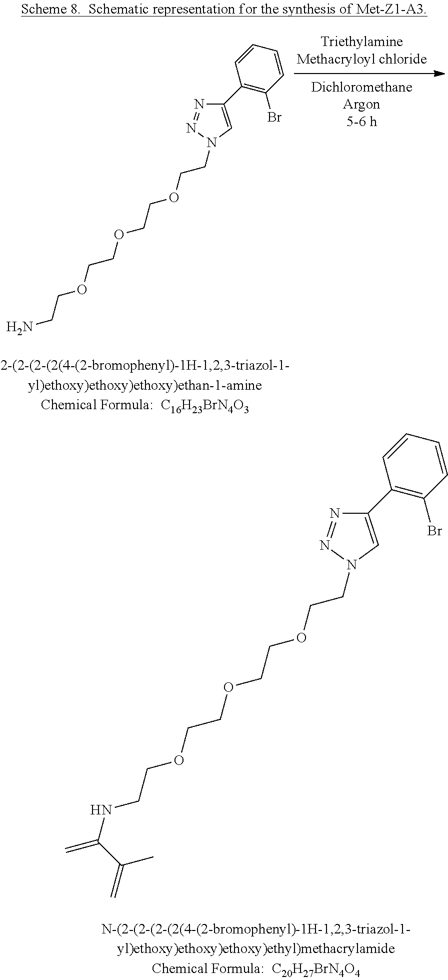 [SMILES: NCCOCCOCCOCCn1cc(-c2ccccc2Br)nn1 ; C=C(C)C(=C)NCCOCCOCCOCCn1cc(-c2ccccc2Br)nn1]

Mass and NMR data. ¹H (600 MHz; CDCl₃): 1.95 (m, 1H, methyl), 3.53 (m, 1H, methylene), 3.55 (m, 1H, methylene), 3.89 (s, 1H, NH₂—CH₂), 4.76 (d, 1H, J=12 Hz, O—CH₂-triazole), 4.85 (d, 1H, J=12 Hz, O—CH₂-triazole), 5.28 (1H, 1 ethylene), 5.74 (d, 1H, 1 ethylene), 7.18 (m, 2H, aromatic), 7.52 (m, 2H, aromatic), 7.73 (m, 2H, aromatic), 7.75 (m, 2H, aromatic), 8.42 (s, 1H, triazole). ¹³C (600 MHz; CDCl₃): 18.6 (CH₃), 39.3 (NH₂CH₂), 50.4 (N—CH₂—C), 69.6 (N—C—CH₂—O), 70.5 (C—CH₂—O), 72 (N—CH₂—CH₂—O), 119.6 (H₂C═C), 122.2 (CH aromatic), 127.2 (CH aromatic), 129.3 (CH aromatic), 130.58 (CH aromatic), 131.4 (CH aromatic), 133.5 (C triazole), 139.9 (CH aromatic), 145.2 (1-ethylene), 168.38 (C, NH—C═O).

### ESI: M+1=469.1.

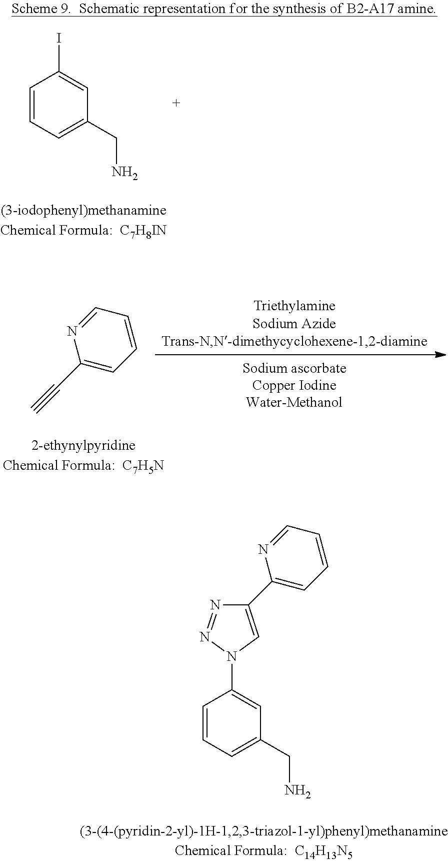

Mass and NMR data: ¹H (600 MHz; DMSO-D₆): 4.17 (s, 2H, NH₂—CH₂-Ph), 7.25 (d, 2H, aromatic), 7.48 (s, 1H, aromatic), 7.52 (s, 2H, aromatic-triazole), 7.87 (s, 1H, aromatic), 7.96 (s, 1H, triazole), 8.25 (s, 1H, aromatic-triazole), 8.59 (s, 2H, amine), 8.65 (s, 1H, aromatic-N). ¹³C (600 MHz; MeOD): 44.2 (CH₂—NH₂), 119.6 (CH aromatic), 121.3 (CH aromatic), 123.7 (CH aromatic), 127.8 (CH aromatic), 129.4 (CH triazole), 137.4 (Cq-N aromatic), 142.3 (Cq-C aromatic), 148.9 (C—N aromatic), 151.2 (C-aromatic).

### ESI: M+1=252.1545

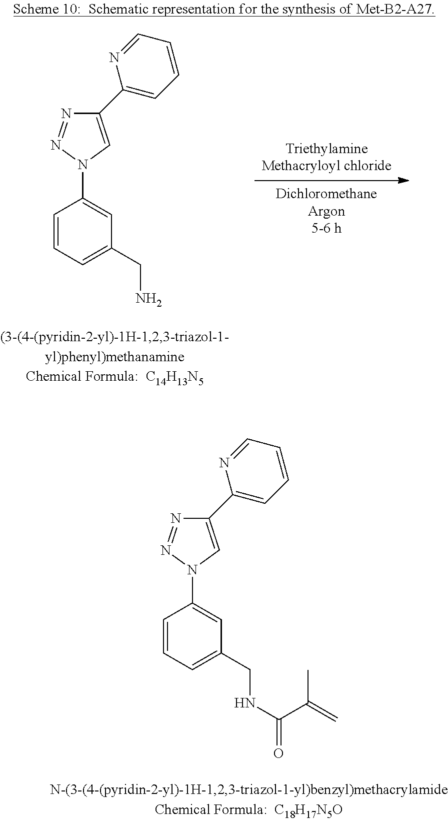 [SMILES: NCc1cccc(-n2cc(-c3ccccn3)nn2)c1 ; C=C(C)C(=O)NCc1cccc(-n2cc(-c3ccccn3)nn2)c1]

Mass and NMR data: ¹H (600 MHz; DMSO-D6): 1.96 (m, 1H, methyl), 4.65 (s, 2H, NH—CH₂-aromatic), 5.56 (s, 1H, ethylene), 5.64 (s, 1H, ethylene), 7.23 (s, 2H, aromatic), 7.48 (s, 1H, aromatic), 7.62 (m, 2H, aromatic), 7.75 (m, 1H, aromatic), 7.83 (s, 1H, triazole), 8.27 (s, 1H, aromatic-triazole), 8.73 (s, 1H, aromatic-N), 8.89 (s, 1H, amide). ¹³C (600 MHz; DMSO-D₆): 18.7 (CH₃, 1-alpha, C—C), 43.1 (CH₂—NH₂), 119.2 (CH₂, ethylene), 120.1 (CH aromatic), 124.3 (CH aromatic), 126.9 (CH aromatic), 131.2 (CH triazole), 137.6 (Cq-N aromatic), 139.7 (Cq-C aromatic), 143.2 (C-ethylene), 148.3 (C—N aromatic), 150.5 (Caromatic), 168.2 (C-amide).

### ESI: M+1=320.1857.

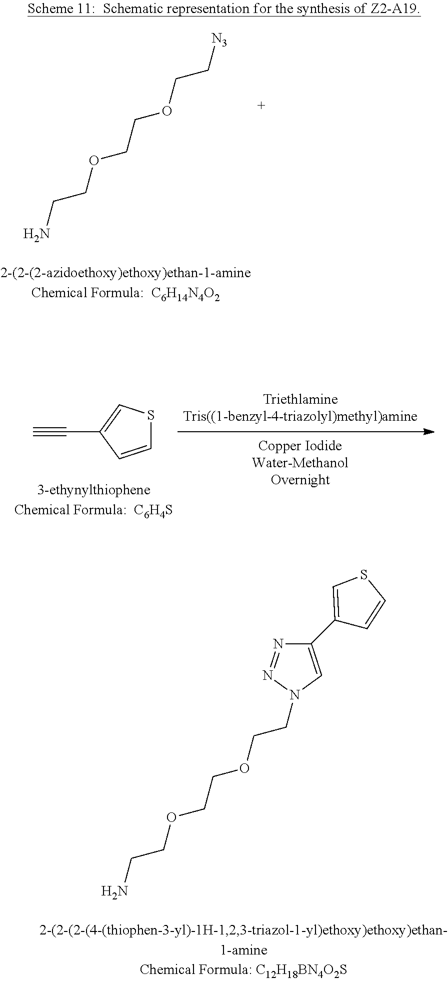

All the compounds, formulations, and methods disclosed and claimed herein can be made and executed without undue experimentation in light of the present disclosure. While the compounds, formulations, and methods of this invention have been described in terms of preferred embodiments, it will be apparent to those of skill in the art that variations may be applied to the compounds, formulations, and methods, as well as in the steps or in the sequence of steps of the method described herein without departing from the concept, spirit, and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references to the extent that they provide exemplary procedural or other details supplementary to those set forth herein, are specifically incorporated herein by reference.
Anderson, Practical Process Research & Development—A Guide for Organic Chemists, 2^{nd} ed., Academic Press, New York, 2012.
Handbook of Pharmaceutical Salts: Properties, and Use, Stahl and Wermuth Eds., Verlag Helvetica Chimica Acta, 2002.
Reagan-Shaw et al., FASEB J., 22(3):659-661, 2008 Smith, March's Advanced Organic Chemistry: Reactions, Mechanisms, and Structure, 7^{th} Ed., Wiley, 2013.
Vegas et al., Nat. Biotechnol., 34(3):345-352, 2016.
Xie et al., Nat. Biomed. Eng., 2(12):894-906, 2018.

What is claimed is:

1. A compound of the formula:
A-L-R₁ (I),
wherein:
A is a polymer;
L is a linker of the formula:
NRₐX₁(CH₂CH₂O)ₒ
wherein:
Rₐ is hydrogen, alkyl_{(C≤6)}, or substituted alkyl_{(C≤6)};
o is 2, 3, 4, or 5; and
X₁ is alkanediyl_{(C≤8)} or substituted alkanediyl_{(C≤8)};
or a linker of the formula:
NR_{b}(CH₂)ₚX₂
wherein:
R_{b} is hydrogen, alkyl_{(C≤6)}, or substituted alkyl_{(C≤6)};
p is 1, 2, or 3; and
X₂ is arenediyl_{(C≤12)} or substituted arenediyl_{(C≤12)};
R₁ is a cycloalkyl_{(C≤12)}; haloaryl_{(C≤12)}; S containing heteroaryl_{(C≤12)}; substituted S-containing heteroaryl_{(C≤12)}; alkyl_{(C≤6)}, haloalkyl_{(C≤6)}, alkenyl_{(C≤6)}, or alkynyl_{(C≤6)} substituted aryl_{(C≤12)}; aralkyl_{(C≤12)}; substituted aralkyl_{(C≤12)}; heterocycloalkyl_{(C≤12)}; substituted heterocycloalkyl_{(C≤12)}; 2-pyridinyl; 3-aminophenyl; 4-alkoxy_{(C≤6)} substituted aryl_{(C≤12)}; or a group of the formula:
X₃OR₂
wherein:
X₃ is alkanediyl_{(C≤8)} or substituted alkanediyl_{(C≤8)};
R₂ is aryl_{(C≤12)} or substituted aryl_{(C≤12)};
or a pharmaceutically acceptable salt thereof.

2. The compound of claim 1 further defined as:
A-L-R₁ (I)
wherein:
A is a polymer
L is a linker of the formula:
NRₐX₁(CH₂CH₂O)ₘ
wherein:
Rₐ is hydrogen, alkyl_{(C≤6)}, or substituted alkyl_{(C≤6)};
m is 2, 3, 4, or 5; and
X₁ is alkanediyl_{(C≤8)} or substituted alkanediyl_{(C≤8)};
R₁ is a cycloalkyl_{(C≤12)}; haloaryl_{(C≤12)}; S containing heteroaryl_{(C≤12)}; substituted S-containing heteroaryl_{(C≤12)}; alkyl_{(C≤6)}, haloalkyl_{(C≤6)}, alkenyl_{(C≤6)}, or alkynye_{(C≤6)} substituted aryl_{(C≤12)}; 3-aminophenyl; 4-alkoxy_{(C≤6)} substituted aryl_{(C≤12)}; or a group of the formula:
X₃OR₂
wherein:
X₃ is alkanediyl_{(C≤8)} or substituted alkanediyl_{(C≤8)};
R₂ is aryl_{(C≤12)} or substituted aryl_{(C≤12)};
or a pharmaceutically acceptable salt thereof.

3. The compound of claim 1 further defined as:
A-L-R₁ (I)
wherein:
A is a polymer
L is a linker of the formula:
NRₐX₁(CH₂CH₂O)ₘ
wherein:
Rₐ is hydrogen, alkyl_{(C≤6)}, or substituted alkyl_{(C≤6)};
m is 2, 3, 4, or 5; and
X₁ is alkanediyl_{(C≤8)} or substituted alkanediyl_{(C≤8)};
or a linker of the formula:
NR_{b}(CH₂)ₙX₂
wherein:
R_{b} is hydrogen, alkyl_{(C≤6)}, or substituted alkyl_{(C≤6)};
n is 1, 2, or 3; and
X₂ is arenediyl_{(C≤12)} or substituted arenediyl_{(C≤12)};
R₁ is a haloaryl_{(C≤12)}; aralkyl_{(C≤12)}; substituted aralkyl_{(C≤12)}; heterocycloalkyl_{(C≤12)}; substituted heterocycloalkyl_{(C≤12)}; 2-pyridinyl; 3-aminophenyl;
or a pharmaceutically acceptable salt thereof.

4. The compound of either claim 1 or claim 2, wherein the polymer comprises one or more sugar repeating units.

5. The compound of claim 4, wherein the repeating unit has a formula: 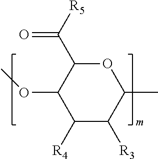
wherein:
R₃ or R₄ are each independently hydrogen or hydroxy;
R₅ is a hydroxy, alkoxy_{(C≤8)}, substituted alkoxy_{(C≤8)}, or a covalent bond to the linker; and
m is a number of repeating units with a molecular weight from about 50,000 Daltons to about 500,000 Daltons.

6. The compound of claim 5, wherein the polymer comprises repeating units of the 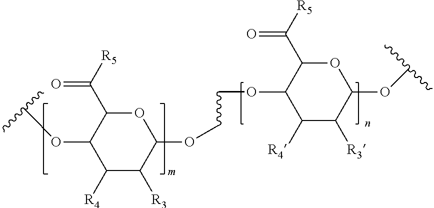
wherein:
R₃, R₃′, R₄, or R_{4′} are each independently hydrogen or hydroxy;
R₅ is a hydroxy, alkoxy_{(C≤8)}, substituted alkoxy_{(C≤8)}, or a covalent bond to the linker;
R₅′ is a covalent bond to the linker; and
m and n result in a number of repeating units with a molecular weight from about 50,000 Daltons to about 500,000 Daltons.

7. The compound according to any one of claims 1-3, wherein the polymer is an acrylate polymer.

8. The compound of claim 7, wherein the polymer is a methacrylate polymer.

9. The compound according to any one of claims 1, 2, and 4-8, wherein o is 2 or 3.

10. The compound of claim 9, wherein o is 2.

11. The compound of claim 9, wherein o is 3.

12. The compound according to any one of claims 1-11, wherein Rₐ is hydrogen.

13. The compound according to any one of claims 1-12, wherein X₁ is alkanediyl_{(C≤6)}.

14. The compound of claim 13, wherein X₁ is —CH₂CH₂—.

15. The compound according to any one of claims 1 and 3-6, wherein R_{b} is hydrogen.

16. The compound according to any one of claims 1, 3-6, and 15, wherein p is 1 or 2.

17. The compound of claim 16, wherein p is 1.

18. The compound according to any one of claims 13, 3-6, and 15-17, wherein X₂ is arenediyl_{(C≤12)}.

19. The compound of claim 18, wherein X₂ is benzenediyl.

20. The compound according to any one of claims 1-19, wherein R₁ is haloaryl_{(C≤12)}.

21. The compound of claim 20, wherein R₁ is chlorophenyl, bromophenyl, or fluorophenyl.

22. The compound of claim 21, wherein R₁ is 2-bromophenyl, 4-chlorophenyl, 2-fluorophenyl, or 4-fluorophenyl.

23. The compound according to any one of claims 1-19, wherein R₁ is 3-aminophenyl.

24. The compound according to any one of claims 1-19, wherein R₁ is an S containing heteroaryl_{(C≤12)} or substituted S containing heteroaryl_{(C≤12)}.

25. The compound of claim 24, wherein R₁ is S containing heteroaryl_{(C≤12)}.

26. The compound of claim 25, wherein R₁ is 2-thiophenyl or 3-thiophenyl.

27. The compound according to any one of claims 1-19, wherein R₁ is cycloalkyl_{(C≤12)}.

28. The compound of claim 27, wherein R₁ is cyclopropyl.

29. The compound according to any one of claims 1-19, wherein R₁ is alkyl_{(C≤6)}, haloalkyl_{(C≤6)}, alkenyl_{(C≤6)}, or alkynye_{(C≤6)} substituted aryl_{(C≤12)}.

30. The compound of claim 29, wherein R₁ is alkyl_{(C≤6)} substituted aryl_{(C≤12)}.

31. The compound of claim 30, wherein R₁ is 3-methylphenyl or 4-methylphenyl.

32. The compound of claim 29, wherein R₁ is a haloalkyl_{(C≤6)} substituted aryl_{(C≤12)}.

33. The compound of claim 32, wherein R₁ is 4-trifluoromethylphenyl.

34. The compound of claim 29, wherein R₁ is an alkynye_{(C≤6)} substituted aryl_{(C≤12)}.

35. The compound of claim 34, wherein R₁ is 3-ethyne-phenyl.

36. The compound according to any one of claims 1-19, wherein R₁ is a group of the formula:
X₃OR₂
wherein:
X₃ is alkanediyl_{(C≤8)} or substituted alkanediyl_{(C≤8)};
R₂ is aryl_{(C≤12)} or substituted aryl_{(C≤12)}.

37. The compound of claim 36, wherein X₃ is alkanediyl_{(C≤8)}.

38. The compound of claim 37, wherein X₃ is —CH₂—.

39. The compound according to any one of claims 36-38, wherein R₂ is substituted aryl_{(C≤12)}.

40. The compound of claim 39, wherein R₂ is 4-aminophenyl.

41. The compound according to any one of claims 1-19, wherein R₁ is 4-alkoxy_{(C≤6)} substituted aryl_{(C≤12)}.

42. The compound of claim 41, wherein R₁ is 4-ethoxyphenyl.

43. The compound according to any one of claims 1-19, wherein R₁ is heterocycloalkyl_{(C≤12)} or substituted heterocycloalkyl_{(C≤12)}.

44. The compound of claim 43, wherein R₁ is heterocycloalkyl_{(C≤12)}.

45. The compound of claim 44, wherein R₁ is thiomorpholine-dioxide.

46. The compound according to any one of claims 1-19, wherein R₁ is aralkyl_{(C≤12)} or substituted aralkyl_{(C≤12)}.

47. The compound of claim 46, wherein R₁ is aralkyl_{(C≤12)}.

48. The compound of claim 47, wherein R₁ is 2-phenylethyl.

49. The compound according to any one of claim 1-48, wherein the compound is further defined as: 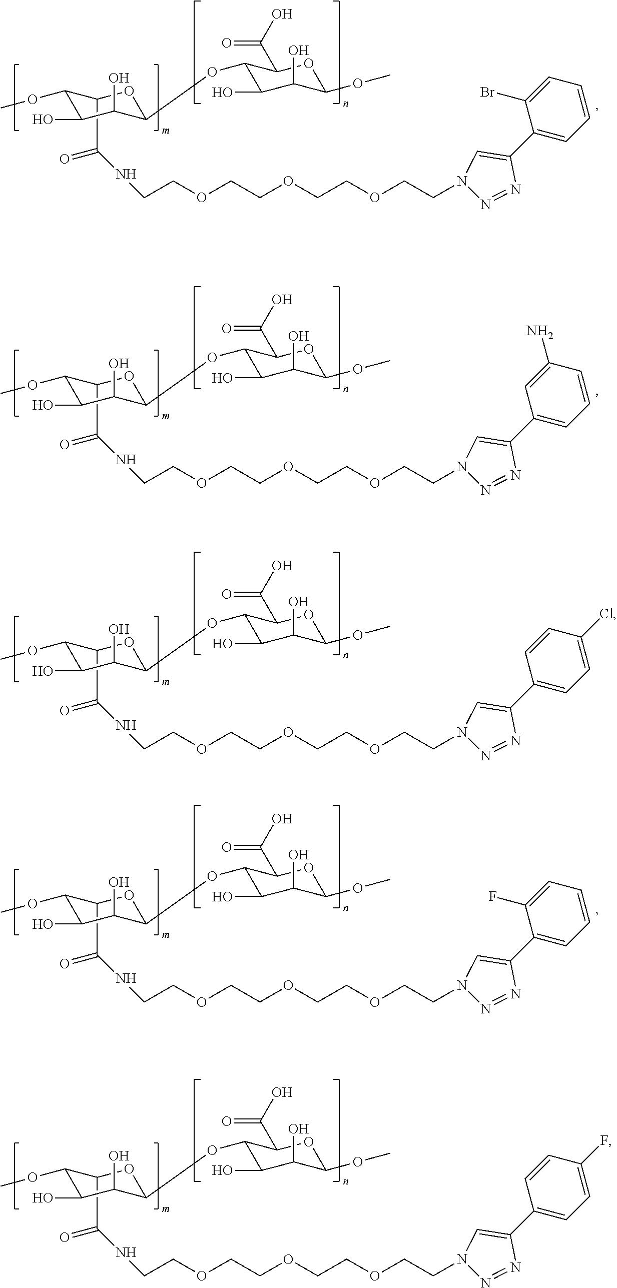 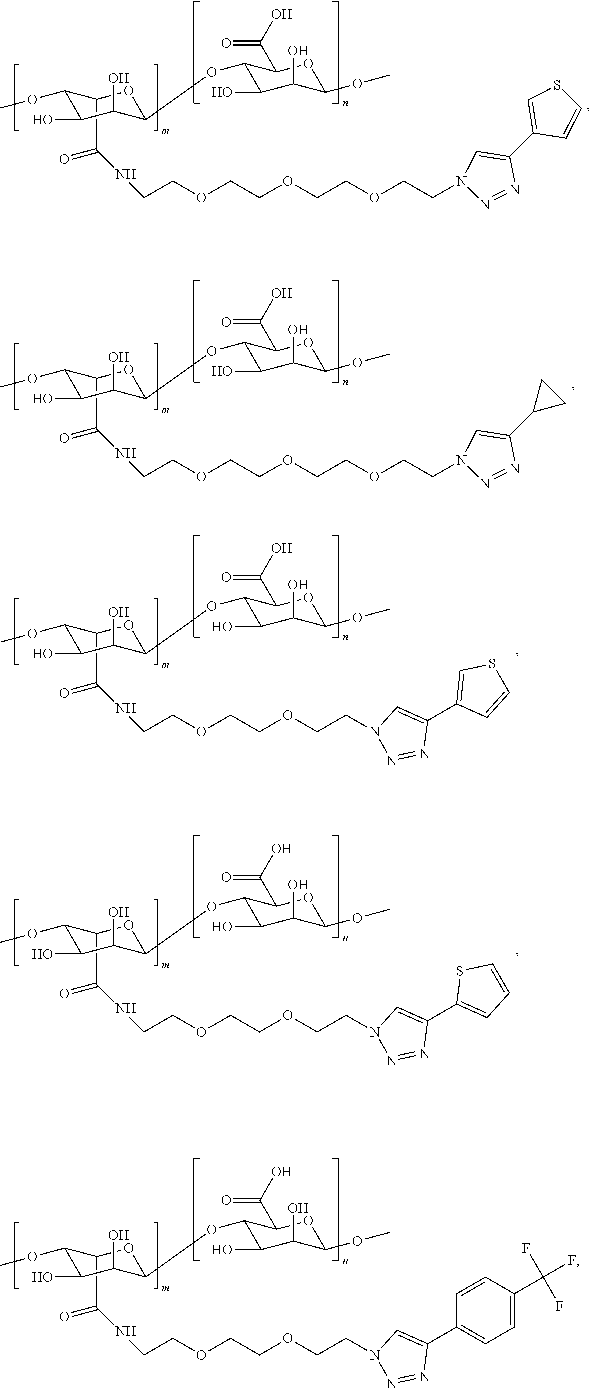 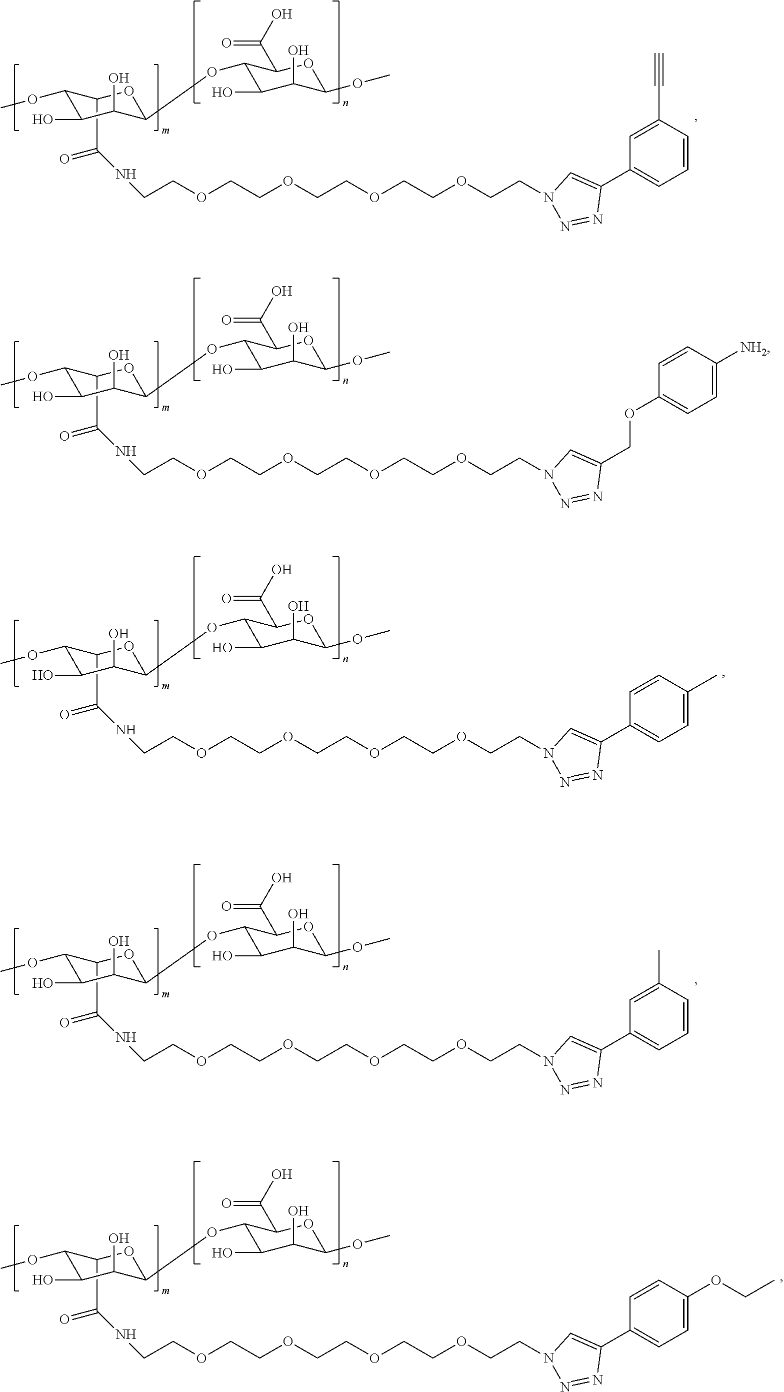 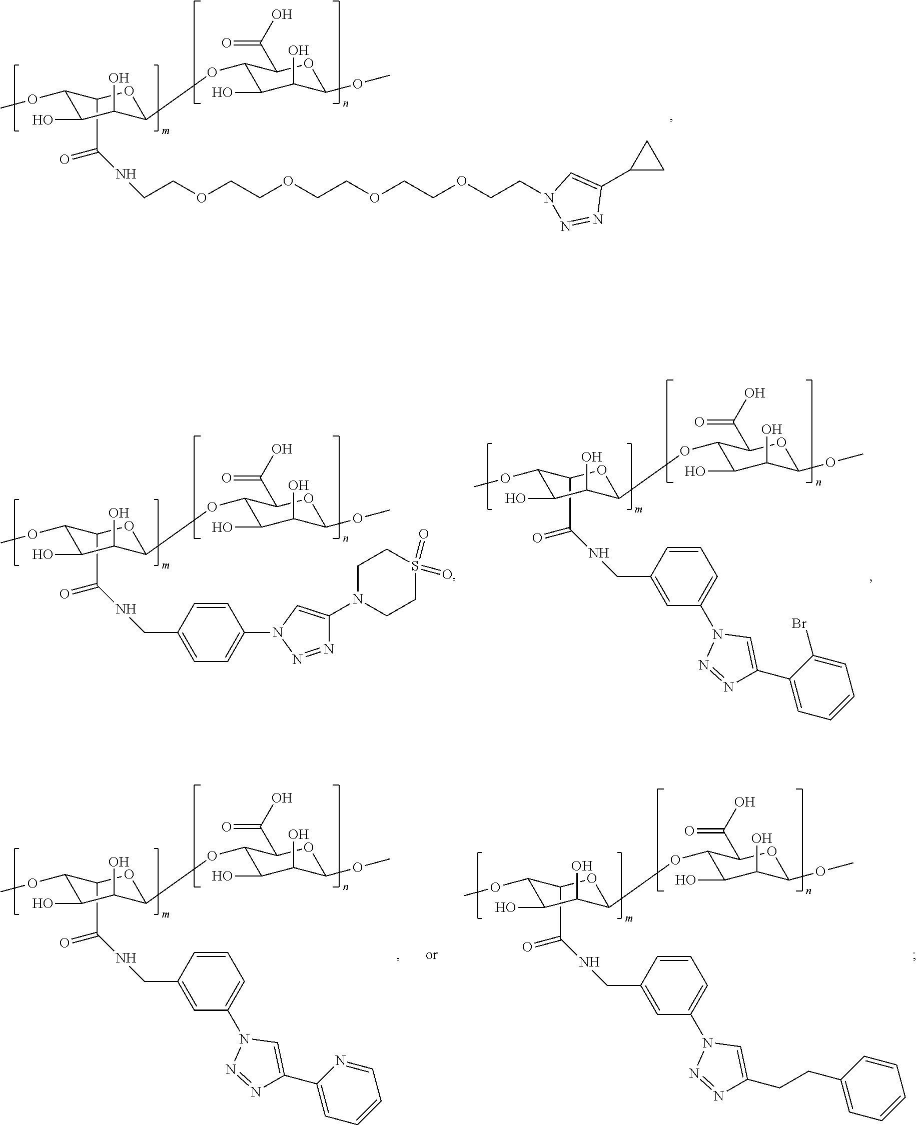
wherein:
m and n result in a number of repeating units with a molecular weight from about 50,000 Daltons to about 500,000 Daltons.

50. A method of detecting fibrosis in a sample comprising exposing the sample to one or more polymers according to any one of claims 1-49 and measuring reactivity.

51. A medical device, wherein the medical device is coated with a compound according to any one of claims 1-49.

52. The medical device of claim 51, wherein the medical device is an implantable device, a cardiac pacemaker, a catheter, a needle injection catheter, a blood clot filter, a vascular transplant, a balloon, a stent transplant, a biliary stent, an intestinal stent, a bronchial stent, an esophageal stent, a ureteral stent, an aneurysm-filling coil or other coil device, a surgical repair mesh, a breast implant, a silicone implant, PDMS, a transmyocardial revascu-larization device, a percutaneous myocardial revasculariza-tion device, a prosthesis, an organ, a vessel, an aorta, a heart valve, a tube, an organ replacement part, an implant, a fiber, a hollow fiber, a membrane, a textile, banked blood, a blood container, a titer plate, an adsorber media, a dialyzer, a connecting piece, a sensor, a valve, an endoscope, a filter, a pump chamber, or another medical device intended to have hemocompatible properties or used in cancer, diabetes, ischemia, anti-bacterial, hemophilia, stroke, blood disorder, or a cytokine therapy involving human engineered cells.

53. The medical device of claim 52, wherein the medical device is a capsule, an implantable polymer block, 3D printed block, 3D printed gel, or a polymer encapsulating device.

54. The medical device of claim 53, wherein the polymer encapsulating device further comprises a shape selected from spheres, squares, noodles, needles, rectangles, and cylindrical.

55. The medical device of claim 53, wherein the implantable capsule is a microcapsule.

56. The medical device of claim 52, wherein the medical device is a catheter.

57. The medical device according to any one of claims 51-56, wherein the medical device results in less fibrosis than a medical device without the coating.

58. The medical device of claim 57, wherein the medical device is immunoprotective compared to a medical device without the coating.

59. The medical device of claim 58, wherein the immunoprotective results in a lower foreign body response.

60. A pharmaceutical composition comprising:
(A) a compound or medical device according to any one of claims 1-58; and
(B) an excipient.

61. The pharmaceutical composition of claim 60, wherein the pharmaceutical composition further comprises biological material.

62. The pharmaceutical composition of claim 61, wherein the biological material is encapsulated in the compound or medical device.

63. The pharmaceutical composition of claim 62, wherein the biological material is cells.

64. The pharmaceutical composition of claim 63, wherein the cells are cells from xenotissue, cells from a cadaver, stem cells, cells derived from stem cells, cells from a cell line, primary cells, reprogrammed cells, reprogrammed stem cells, cells derived from reprogrammed stem cells, genetically engineered cells, or a combination thereof.

65. The pharmaceutical composition of claim 63, wherein the cells are human cells.

66. The pharmaceutical composition of claim 63, wherein the cells are insulin-producing cells.

67. The pharmaceutical composition of claim 66, wherein the cells are pancreatic islet cells.

68. The pharmaceutical composition according to any one of claims 60-67, wherein the compound is cross-linked.

69. The pharmaceutical composition of claim 68, wherein the cross-linked compound is covalently cross-linked.

70. A method of treating or preventing a disease or disorder comprising administering to a patient in need thereof a compound, a medical device, or a pharmaceutical composition according to any one of claims 1-69.

71. The method of claim 70, wherein the method results in a lower foreign body response.

72. The method of claim 70 or claim 71, wherein the method results in less fibrosis.


SEQUENCE LISTING (86 sequences)

SEQ ID NO: 1 - DNA, 21 bp, Artificial sequence
  Synthetic primer
cgcttccgctgcccagagact

SEQ ID NO: 2 - DNA, 26 bp, Artificial sequence
  Synthetic primer
tataggtggtttcgtggatgcccgct

SEQ ID NO: 3 - DNA, 21 bp, Artificial sequence
  Synthetic primer
catgttcagctttgtggacct

SEQ ID NO: 4 - DNA, 20 bp, Artificial sequence
  Synthetic primer
gcagctgacttcagggatgt

SEQ ID NO: 5 - DNA, 22 bp, Artificial sequence
  Synthetic primer
gcttctttgcagctccttcgtt

SEQ ID NO: 6 - DNA, 20 bp, Artificial sequence
  Synthetic primer
cggagccgttgtcgacgacc

SEQ ID NO: 7 - DNA, 41 bp, Artificial sequence
  Synthetic primer
ccaaagtcgttaagctgccaaaccaatgggagtcactgctg

SEQ ID NO: 8 - DNA, 44 bp, Artificial sequence
  Synthetic primer
tatgtccgtcgttgcgaagtgcctggacatcagtgtcctcatct

SEQ ID NO: 9 - DNA, 48 bp, Artificial sequence
  Synthetic primer
ccaaagtcgttaagctgccaacctgaatgtcagttttgttagagcaac

SEQ ID NO: 10 - DNA, 45 bp, Artificial sequence
  Synthetic primer
tatgtccgtcgttgcgaagtgacagggtgatgtaaaagtgtctga

SEQ ID NO: 11 - DNA, 48 bp, Artificial sequence
  Synthetic primer
ccaaagtcgttaagctgccaacagacttaatcaaagcccttgaaaaga

SEQ ID NO: 12 - DNA, 43 bp, Artificial sequence
  Synthetic primer
tatgtccgtcgttgcgaagtgactcatggcaacttgcttctca

SEQ ID NO: 13 - DNA, 48 bp, Artificial sequence
  Synthetic primer
ccaaagtcgttaagctgccaacctcccatagtgattcttatgaagtca

SEQ ID NO: 14 - DNA, 45 bp, Artificial sequence
  Synthetic primer
tatgtccgtcgttgcgaagtgggataaccttgtgtgcatgtcact

SEQ ID NO: 15 - DNA, 44 bp, Artificial sequence
  Synthetic primer
ccaaagtcgttaagctgccaaccacactctgcctctcatggtat

SEQ ID NO: 16 - DNA, 44 bp, Artificial sequence
  Synthetic primer
tatgtccgtcgttgcgaagtgaggcaagtaagcagacaatagca

SEQ ID NO: 17 - DNA, 45 bp, Artificial sequence
  Synthetic primer
ccaaagtcgttaagctgccaatccgcaaaacctacaatctctgaa

SEQ ID NO: 18 - DNA, 46 bp, Artificial sequence
  Synthetic primer
tatgtccgtcgttgcgaagtgcagtatagctgaggtaaccacagta

SEQ ID NO: 19 - DNA, 53 bp, Artificial sequence
  Synthetic primer
ccaaagtcgttaagctgccaacttgtatatagacggtaaaataaacaccaaga

SEQ ID NO: 20 - DNA, 48 bp, Artificial sequence
  Synthetic primer
tatgtccgtcgttgcgaagtgctggacagttaccttattcaaacatca

SEQ ID NO: 21 - DNA, 44 bp, Artificial sequence
  Synthetic primer
ccaaagtcgttaagctgccaattcctgcttccagacatgaatca

SEQ ID NO: 22 - DNA, 42 bp, Artificial sequence
  Synthetic primer
tatgtccgtcgttgcgaagtgcaccatacctccatgacacca

SEQ ID NO: 23 - DNA, 41 bp, Artificial sequence
  Synthetic primer
ccaaagtcgttaagctgccaactctctgcctgcaggatgtg

SEQ ID NO: 24 - DNA, 43 bp, Artificial sequence
  Synthetic primer
tatgtccgtcgttgcgaagtggcgtttctcactggtctcagat

SEQ ID NO: 25 - DNA, 43 bp, Artificial sequence
  Synthetic primer
ccaaagtcgttaagctgccaaactaagagtgcagagcctggaa

SEQ ID NO: 26 - DNA, 44 bp, Artificial sequence
  Synthetic primer
tatgtccgtcgttgcgaagtgtggggtttgtgaacaagagtaga

SEQ ID NO: 27 - DNA, 47 bp, Artificial sequence
  Synthetic primer
ccaaagtcgttaagctgccaacactttatcagacacagttatgtgct

SEQ ID NO: 28 - DNA, 48 bp, Artificial sequence
  Synthetic primer
tatgtccgtcgttgcgaagtggcccacatttagaatttagaggtaact

SEQ ID NO: 29 - DNA, 47 bp, Artificial sequence
  Synthetic primer
ccaaagtcgttaagctgccaactatctgcaggattgtgttcaatgta

SEQ ID NO: 30 - DNA, 44 bp, Artificial sequence
  Synthetic primer
tatgtccgtcgttgcgaagtgagcaaaccactggggaaaatact

SEQ ID NO: 31 - DNA, 46 bp, Artificial sequence
  Synthetic primer
ccaaagtcgttaagctgccaactctctagagtgcagattggtagaa

SEQ ID NO: 32 - DNA, 45 bp, Artificial sequence
  Synthetic primer
tatgtccgtcgttgcgaagtgcaagtgagagaccaaggaaaacaa

SEQ ID NO: 33 - DNA, 51 bp, Artificial sequence
  Synthetic primer
ccaaagtcgttaagctgccaacaaagttgataaattaaaggactaaggcac

SEQ ID NO: 34 - DNA, 46 bp, Artificial sequence
  Synthetic primer
tatgtccgtcgttgcgaagtgttaaatcctgcctctactacttgct

SEQ ID NO: 35 - DNA, 44 bp, Artificial sequence
  Synthetic primer
ccaaagtcgttaagctgccaacattctgtctgggatgaggtgat

SEQ ID NO: 36 - DNA, 54 bp, Artificial sequence
  Synthetic primer
tatgtccgtcgttgcgaagtgaacagttatatgaaaaaaatgctcaatatcact

SEQ ID NO: 37 - DNA, 42 bp, Artificial sequence
  Synthetic primer
ccaaagtcgttaagctgccaatgaaagacgtcacagcaaggt

SEQ ID NO: 38 - DNA, 39 bp, Artificial sequence
  Synthetic primer
tatgtccgtcgttgcgaagtgcggggaccaggagcaaag

SEQ ID NO: 39 - DNA, 45 bp, Artificial sequence
  Synthetic primer
ccaaagtcgttaagctgccaatgtaggagagattgggctagagag

SEQ ID NO: 40 - DNA, 47 bp, Artificial sequence
  Synthetic primer
tatgtccgtcgttgcgaagtgctctacctgggaaatctcattcattc

SEQ ID NO: 41 - DNA, 51 bp, Artificial sequence
  Synthetic primer
ccaaagtcgttaagctgccaactaacttcctaactaaaactttacagtgga

SEQ ID NO: 42 - DNA, 46 bp, Artificial sequence
  Synthetic primer
tatgtccgtcgttgcgaagtgtcagtagaactttgaagggtacaca

SEQ ID NO: 43 - DNA, 45 bp, Artificial sequence
  Synthetic primer
ccaaagtcgttaagctgccaagcacgtagatgaaattgccccata

SEQ ID NO: 44 - DNA, 50 bp, Artificial sequence
  Synthetic primer
tatgtccgtcgttgcgaagtgacttcctacttagccctttagaaatgtaa

SEQ ID NO: 45 - DNA, 49 bp, Artificial sequence
  Synthetic primer
ccaaagtcgttaagctgccaaggaaaatatgtctaaaaaggctctggag

SEQ ID NO: 46 - DNA, 44 bp, Artificial sequence
  Synthetic primer
tatgtccgtcgttgcgaagtgccagtgcagtgtttctcaaactc

SEQ ID NO: 47 - DNA, 47 bp, Artificial sequence
  Synthetic primer
ccaaagtcgttaagctgccaagtttgttctaaggttcatctggtgat

SEQ ID NO: 48 - DNA, 52 bp, Artificial sequence
  Synthetic primer
tatgtccgtcgttgcgaagtggctcatgaagaaaataatccttatggtaatc

SEQ ID NO: 49 - DNA, 44 bp, Artificial sequence
  Synthetic primer
ccaaagtcgttaagctgccaatgtcccactttttacctcccttc

SEQ ID NO: 50 - DNA, 48 bp, Artificial sequence
  Synthetic primer
tatgtccgtcgttgcgaagtgcttcatggaggagatagtaactaaggt

SEQ ID NO: 51 - DNA, 44 bp, Artificial sequence
  Synthetic primer
ccaaagtcgttaagctgccaatgtgctacgacagagctaagtac

SEQ ID NO: 52 - DNA, 45 bp, Artificial sequence
  Synthetic primer
tatgtccgtcgttgcgaagtgtggtcagcttaaatagctactgct

SEQ ID NO: 53 - DNA, 40 bp, Artificial sequence
  Synthetic primer
ccaaagtcgttaagctgccaaccccggatgtcagggaatg

SEQ ID NO: 54 - DNA, 43 bp, Artificial sequence
  Synthetic primer
tatgtccgtcgttgcgaagtggtgagtatgcacgtctccatct

SEQ ID NO: 55 - DNA, 40 bp, Artificial sequence
  Synthetic primer
ccaaagtcgttaagctgccaaccaggcaccactgctttgt

SEQ ID NO: 56 - DNA, 46 bp, Artificial sequence
  Synthetic primer
tatgtccgtcgttgcgaagtggcaaggggacagaaatttgcttatc

SEQ ID NO: 57 - DNA, 49 bp, Artificial sequence
  Synthetic primer
ccaaagtcgttaagctgccaaaccttgtcaagaacctaaatagtgagaa

SEQ ID NO: 58 - DNA, 41 bp, Artificial sequence
  Synthetic primer
tatgtccgtcgttgcgaagtgtgggagagttcactgcacct

SEQ ID NO: 59 - DNA, 51 bp, Artificial sequence
  Synthetic primer
ccaaagtcgttaagctgccaacgtgggctagtcaagaatataaaatgttag

SEQ ID NO: 60 - DNA, 42 bp, Artificial sequence
  Synthetic primer
tatgtccgtcgttgcgaagtgcatgcaggtggtgtgaatctc

SEQ ID NO: 61 - DNA, 45 bp, Artificial sequence
  Synthetic primer
ccaaagtcgttaagctgccaatgtgtggctcagtataccacttag

SEQ ID NO: 62 - DNA, 50 bp, Artificial sequence
  Synthetic primer
tatgtccgtcgttgcgaagtgctcaagccatgtcatattttcaaatagac

SEQ ID NO: 63 - DNA, 49 bp, Artificial sequence
  Synthetic primer
ccaaagtcgttaagctgccaagcacatcatacattatttctgttgctat

SEQ ID NO: 64 - DNA, 42 bp, Artificial sequence
  Synthetic primer
tatgtccgtcgttgcgaagtgagagcccacttagcatctcca

SEQ ID NO: 65 - DNA, 42 bp, Artificial sequence
  Synthetic primer
ccaaagtcgttaagctgccaagaaatattgctggggtcagcg

SEQ ID NO: 66 - DNA, 48 bp, Artificial sequence
  Synthetic primer
tatgtccgtcgttgcgaagtgggagggtttaaggtgttttatgttttg

SEQ ID NO: 67 - DNA, 28 bp, Artificial sequence
  Synthetic primer
atacgtgccaaagtcgttaagctgccaa

SEQ ID NO: 68 - DNA, 28 bp, Artificial sequence
  Synthetic primer
tgaagttccaaagtcgttaagctgccaa

SEQ ID NO: 69 - DNA, 28 bp, Artificial sequence
  Synthetic primer
tgattagccaaagtcgttaagctgccaa

SEQ ID NO: 70 - DNA, 28 bp, Artificial sequence
  Synthetic primer
ctaatcaccaaagtcgttaagctgccaa

SEQ ID NO: 71 - DNA, 28 bp, Artificial sequence
  Synthetic primer
atgacgcccaaagtcgttaagctgccaa

SEQ ID NO: 72 - DNA, 28 bp, Artificial sequence
  Synthetic primer
gtaatgtccaaagtcgttaagctgccaa

SEQ ID NO: 73 - DNA, 28 bp, Artificial sequence
  Synthetic primer
aaacttcccaaagtcgttaagctgccaa

SEQ ID NO: 74 - DNA, 28 bp, Artificial sequence
  Synthetic primer
tccgagaccaaagtcgttaagctgccaa

SEQ ID NO: 75 - DNA, 28 bp, Artificial sequence
  Synthetic primer
gtcaatctatgtccgtcgttgcgaagtg

SEQ ID NO: 76 - DNA, 28 bp, Artificial sequence
  Synthetic primer
cgctatgtatgtccgtcgttgcgaagtg

SEQ ID NO: 77 - DNA, 28 bp, Artificial sequence
  Synthetic primer
accgatttatgtccgtcgttgcgaagtg

SEQ ID NO: 78 - DNA, 28 bp, Artificial sequence
  Synthetic primer
aacaccgtatgtccgtcgttgcgaagtg

SEQ ID NO: 79 - DNA, 28 bp, Artificial sequence
  Synthetic primer
ctcggaatatgtccgtcgttgcgaagtg

SEQ ID NO: 80 - DNA, 28 bp, Artificial sequence
  Synthetic primer
gactgattatgtccgtcgttgcgaagtg

SEQ ID NO: 81 - DNA, 28 bp, Artificial sequence
  Synthetic primer
tactgcgtatgtccgtcgttgcgaagtg

SEQ ID NO: 82 - DNA, 28 bp, Artificial sequence
  Synthetic primer
aactggctatgtccgtcgttgcgaagtg

SEQ ID NO: 83 - DNA, 28 bp, Artificial sequence
  Synthetic primer
atcggtctatgtccgtcgttgcgaagtg

SEQ ID NO: 84 - DNA, 28 bp, Artificial sequence
  Synthetic primer
gccagtttatgtccgtcgttgcgaagtg

SEQ ID NO: 85 - DNA, 28 bp, Artificial sequence
  Synthetic primer
cacgtattatgtccgtcgttgcgaagtg

SEQ ID NO: 86 - DNA, 28 bp, Artificial sequence
  Synthetic primer
cgcatgttatgtccgtcgttgcgaagtg